(19) United States
(12) Patent Application Publication
(10) Pub. No.: US 2026/0148827 A1
(43) Pub. Date: May 28, 2026

(54) MOTION ANALYSIS SYSTEMS AND METHODS OF USE THEREOF

(71) Applicant: Highland Instruments, Inc., Somerville, MA (US)

(72) Inventors: Timothy Andrew Wagner, Somerville, MA (US); Laura Dipietro, Cambridge, MA (US); Uri Tzvi Eden, Somerville, MA (US)

(21) Appl. No.: 19/121,733

(22) PCT Filed: Oct. 16, 2023
(86) PCT No.: PCT/US2023/077005

§ 371 Date: Apr. 16, 2025

Related U.S. Application Data
(60) Provisional application No. 63/417,310, filed on Oct. 18, 2022.
(60) Provisional application No. 63/437,746, filed on Jan. 8, 2023.
(60) Provisional application No. 63/437,750, filed on Jan. 9, 2023.

(51) Int. Cl. G16H 20/30; A61B 5/00; A61B 5/11; G16H 50/20

(52) U.S. Cl. CPC G16H 20/30; A61B 5/1124; A61B 5/4082; G16H 50/20

(57) ABSTRACT

The disclosure generally relates to motion analysis systems and methods of use thereof. In certain aspects, the system includes an image capture device, at least one accelerometer, and a central processing unit (CPU) with storage coupled thereto for storing instructions that when executed by the CPU cause the CPU to receive a first set of motion data from the image capture device related to at least one joint of a subject while the subject is performing a task and receive a second set of motion data from the accelerometer related to the at least one joint of the subject while the subject is performing the task. The CPU also calculates kinematic and/or kinetic information about the at least one joint of a subject from a combination of the first and second sets of motion data, and outputs the kinematic and/or kinetic information for purposes of assessing a movement disorder.

### CROSS REFERENCE TO RELATED APPLICATIONS

This application claims the benefit of and priority to U.S. Provisional Patent Application Nos. 63/417,310 filed Oct. 18, 2022, 63/437,746 filed Jan. 8, 2023, and 63/437,750 filed Jan. 9, 2023, the contents of each are incorporated by reference herein in their entireties.

### GOVERNMENT SUPPORT

This disclosure was made with Government support under Grant Numbers AG055360, AR076885, DA049685, DK117710, NS110237, and NS113737 awarded by the National Institutes of Aging (NIA), National Institute of Arthritis and Musculoskeletal and Skin Diseases, National Institutes of Drug Abuse, National Institute of Diabetes and Digestive and Kidney Diseases, and the National Institute of Neurological Disorders and Stroke (NINDS) of the National Institute of Health (NIH). The Government has certain rights in this disclosure.

### FIELD OF THE INVENTION

The disclosure generally relates to methods for assessing, determining a management plan, and/or optimizing care for a patient with a movement disorder using a motion analysis system.

### BACKGROUND

Parkinson's disease (PD) is a chronic and progressive movement disorder. Nearly one million people in the United States are living with Parkinson's disease. Parkinson's disease involves malfunction and death of vital nerve cells in the brain, called neurons. Parkinson's disease affects neurons in an area of the brain known as the substantia nigra. Some of those dying neurons produce dopamine, a chemical that sends messages to the part of the brain that controls movement and coordination. As Parkinson's disease progresses, the amount of dopamine produced in brain areas decreases, leaving a person unable to control movement normally. Parkinson's disease can also be defined as a disconnection syndrome, in which PD-related disturbances in neural connections among subcortical and cortical structures can negatively impact the motor systems of Parkinson's disease patients and further lead to deficits in cognition, perception, and other neuropsychological aspects seen with the disease (Cronin-Golomb, Neuropsychology review. 2010;20(2):191-208. doi: 10.1007/s11065-010-9128-8. PubMed PMID: 20383586; PubMed Central PMCID: PMC2882524).

While significant advancements have been made in the treatment of PD, limited progress has been made in disease diagnoses and evaluation. Diagnosis is primarily based on physical exam findings of characteristic PD motor symptoms. However, early diagnosis of PD and Parkinsonian syndromes is quite challenging, and up to 25% of new patients can go misdiagnosed or undiagnosed. Furthermore, diagnostic accuracy at primary care facilities, the principal site in patient evaluations, and the evaluations made by general practitioners and/or nurse can be limited in comparison to evaluations made by movement disorder specialists. It is postulated that movement disorders experts assess PD symptoms in their entirety across the motor system, with pattern recognition capabilities which non-expert clinicians lack. Beyond initial diagnosis, disease progression is tracked using coarse clinical scales, such as the Unified Parkinson's Disease Rating Scale (UPDRS), which suffer from limited resolution and high intra-and inter-rater variability. Furthermore, PD motor symptoms fluctuate throughout the day, yet clinical rating scales only provide single time point assessments, and therefore might not reflect the true state of the disease. While ultimately these diagnostic limitations impact the patient's course of care, they also necessitate significant resources to conduct PD clinical trials (e.g., large sample size) and limit therapy customization.

Numerous rating scales exist for diagnosing Parkinson's disease. The Unified Parkinson's Disease Rating Scale (UPDRS) is the most commonly used scale in the clinical study of Parkinson's Disease. The UPDRS is made up of the following sections: evaluation of Mentation, behavior, and mood; self-evaluation of the activities of daily life (ADLs) including speech, swallowing, handwriting, dressing, hygiene, falling, salivating, turning in bed, walking, cutting food; clinician-scored monitored motor evaluation; Hoehn and Yahr staging of severity of Parkinson disease; and Schwab and England ADL scale.

A problem with the UPDRS is that it is highly subjective because the sections of the UPDRS are evaluated by interview and clinical observation from a team of different specialists. Some sections require multiple grades assigned to each extremity. Because of subjective nature of the UPDRS, it is sometimes difficult to accurately assess a subject. Furthermore, since the UPDRS is based on human observation, it can be difficult to notice subtle changes in disease progression over time. Finally, the nature of UPDRS measurements, based on subjective clinician evaluations, leads to variability due to observer and observer state.

Another disease or condition which can impact patient movement is stroke. Most survivors suffer motor deficits (˜70%) and require rehabilitation. Tools to predict the extent to which a patient can recover their motor abilities or even to measure current abilities are needed. Furthermore, conventional clinical assessments (e.g., NIH Stroke Scale, Fugl-Meyer (FM)) (Fugl-Meyer et al., The post-stroke hemiplegic patient. 1. a method for evaluation of physical performance, Scand J Rehabil Med, 1975; Spilker et al. Using the NIH Stroke Scale to assess stroke patients. The NINDS rt-PA Stroke Study Group, J Neurosci Nurs, 1997) that power prognosis are highly dependent on the care provider/point of care and are often reduced to even coarser prognostic scales (e.g., Orpington Prognostic Scale (OPS)) (Rieck & Moreland, The Orpington Prognostic Scale for patients with stroke: reliability and pilot predictive data for discharge destination and therapeutic services, Disability and rehabilitation, 2005).

### SUMMARY

This disclosure includes a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment. In a particular embodiment, the system could integrate sensors such as, but not limited to motion capture camera(s), force sensor(s), inertial sensor(s) (e.g., accelerometer /d/ gyroscope), galvanic sensor(s), heart rate monitor(s), respiratory sensor(s), blood oxygen sensor(s), metabolic sensors, electrophysiology sensors, and/or event trigger device(s) and/or methods to objectively and quantitatively measure patient movement, patient metabolism, and/or patient biofunction. The system could use a single sensor at a time or multiple sensors at a time. The sensors can be for example fixed in place, be portable, be mobile, placed on a patient, placed on a care giver (e.g., person conducting assessment), be part of an external device that the patient or caregiver carries with them (e.g., accelerometers in a cell phone) or uses as part of a movement and/or assessment (e.g., writing utensil), and/or placed in a wearable item. The sensor can be embedded or woven in garments/clothing or fabrics, be part of or integrated with an adhesive, placed in device carried by the user (e.g., cell phone), or directly worn or placed on the user. The sensors can be integrated and synchronized via a computing device or computing devices (e.g., integrated chip-based device, computer, tablet, cell phone). The sensors can be connected via wires, wirelessly, and/or via a memory disk that can be transferred between a sensor and an external computing device and/or between sensors. The patient movement data can be transferred real-time (as its being recorded) and/or after patient assessments are taken and then transferred to an external system for storage and/or analysis. The computing device can be an external device and/or part of a sensor or sensors. The computing device could control the sensors and synchronize and/or integrate the various sensor information that is recorded from the patient and/or care provider. The system can provide feedback to the user, and vice versa, such as through a keyboard, pointing device screen, or any such method (e.g., feedback provided to the user can be any form of sensory feedback, (e.g., visual feedback, auditory feedback, or tactile feedback), and input from the user can be received in any form, including acoustic, speech, gesture(s) (e.g., via a man-machine interface that recognizes gestures), neural signals (e.g., via a brain-machine interface), or tactile input). The system includes software to derive quantitative movement kinematic/kinetic-based motor evaluations; statistical and/or machine learning algorithms for data reduction, data modeling, predictions of clinical scales and/or prognostic potential for disease recovery, predictions of clinical scales and/or prognostic potential for response for therapy and/or assessments that can guide a tuned response to therapy and/or methodologies to dose and or tune a therapy. The system could store data and complete computational analysis via cloud a based network. The system can be a single computer system with internal connected sensors and/or external connected sensors and or multiple integrated computer systems with internal connected sensors and/or external connected sensors (whereby integration of computer systems can be completed via wired connections, wireless communication, and/or the transfer of data through external mechanisms (e.g., external storage devices and/or or intermediary communications and/or storage devices). The system can be based on or make use of cloud-based computing and/or multiple networks connected computer systems. The computational system(s) can be integrated with a database of patient clinical and/or demographic data, which can be used as part of the statistical and/or machine learning calculations and assessments (additional types of databases, analyses methods, and data types which can be integrated with the method can be found in (DIPIETRO L, GONZALEZ-MEGO P, RAMOS-ESTEBANEZ C, ZUKOWSKI LH, MIKKILINENI R, RUSHMORE RJ, WAGNER T. THE EVOLUTION OF BIG DATA IN NEUROSCIENCE AND NEUROLOGY. J BIG DATA. 2023;10(1):116. DOI: 10.1186/S40537-023-00751-2. EPUB 2023 Jul. 10. PMID: 37441339). Generally, Big Data (or big data) primarily refers to data sets that are too large or complex to be dealt with by traditional data-processing application software (e.g., data with many entries offer greater statistical power, while data with higher complexity may lead to a higher false discovery rate) or data that complies definitions such as the 5 V definitions (where the 5 V are Volume, Variety, Velocity, Veracity and Value), (or any subsets of the 5Vs) as detailed in THE EVOLUTION OF BIG DATA IN NEUROSCIENCE AND NEUROLOGY incorporated herein. The system itself can be designed such that the statistical and/or machine learning calculations and assessments can continually improve their capability (e.g., accuracy of predictions, resolution of assessments) based on the access to database(s) of patient clinical and/or demographic data and/or past calculation or assessment results. The system itself can be employed directly in person and/or remotely such as via telehealth-based assessments. In certain embodiments, the system can further be integrated directly with a patient billing and reimbursement databases to see that its use is properly compensated and/or used to regulate use of the system.

Aspects of this disclosure include motion analysis systems that can objectively evaluate a subject for Parkinson's disease, or any type of movement disorder, based on motion data obtained from one or more joints of a subject. Aspects of the disclosure are accomplished with an image capture device, at least one external body motion sensor, and a computer including processing software that can integrate the data received from the image capture device and the external body motion sensor. Particularly, the processor receives a first set of motion data from the image capture device related to at least one joint of a subject while the subject is performing a task and receives a second set of motion data from the external body motion sensor (e.g., an accelerometer) related to the at least one joint of the subject while the subject is performing the task. The processor then calculates kinematic and/or kinetic information about the at least one joint of the subject from a combination of the first and second sets of motion data, and outputs the kinematic and/or kinetic information for purposes of assessing a movement disorder. In that manner, human observation is removed from the evaluation of a patient, and a standard set of diagnostic measurements is provided for evaluating patients. That provides a unified and accepted assessment rating system across a patient population, which allows for uniform assessment of the patient population. Additionally, since systems of the disclosure are significantly more sensitive than human observation, subtle changes in disease progression can be monitored and more accurate stratification of a patient population can be achieved. In addition to information from body locations where at least two points of a skeleton (and/or other body tissues) join, joint information can include information from body, body components, and/or limb positions (such as a location on a single skeletal bone), and/or inferred and/or calculated body positions (such as for example the center of the forearm). Other types of data can be integrated with systems of the disclosure to give a fuller picture of a subject. For example, systems of the disclosure can also include a force plate, which can record balance data of the subject. In such embodiments, the processor receives balance data from the force plate, calculates kinematic and/or kinetic information about the at least one joint of a subject from a combination of the first and second sets of motion data and the balance data, and outputs the kinematic and/or kinetic information for purposes of assessing a movement disorder. Other types of data that are useful to obtain are eye tracking data and voice data. Accordingly, systems of the disclosure may also include a device for eye tracking and/or a device for voice tracking. In such embodiments, the processor receives balance data, voice data, and/or eye data, calculates kinematic and/or kinetic information about the at least one joint of a subject from a combination of the first and second sets of motion data, the balance data, the eye tracking data, and/or voice data, and outputs the kinematic and/or kinetic information for purposes of assessing a movement disorder. In other embodiments, systems of the disclosure include a gyroscope and the second set of motion data further includes gyroscopic data. In certain embodiments, the kinematic and/or kinetic information includes information about velocity of the joint. In certain embodiments, the processor renders received data from the image capture device as a skeletal joint map. In other embodiments, software of the image capture device renders received video data as a skeletal joint map and then sends the skeletal joint map to the processor.

There are any number of tasks that the subject can perform while being evaluated by the motion analysis system. Exemplary tasks include discrete flexion of a limb, discrete extension of a limb, continuous flexion of a limb, continuous extension of a limb, rotation of a limb, opening of a hand, closing of a hand, walking, standing, or any combination thereof.

Typically, the subject is afflicted with a movement disorder. Exemplary movement disorders include diseases which affect a person's control or generation of movement, whether at the site of a joint (e.g., direct trauma to a joint where damage to the joint impacts movement), in neural and/or muscle/skeletal circuits (such as parts of the basal ganglia in Parkinson's Disease), or in both (such as in a certain pain syndrome chronic pain syndrome where for instance a joint could be damaged, generating pain signals, that in turn are associated with change in neural activity caused by the pain). Exemplary movement disorders include Parkinson's disease, Parkinsonism (aka., Parkinsonianism which includes Parkinson's Plus disorders such as Progressive Supranuclear Palsy, Multiple Systems Atrophy, and/or Corticobasal syndrome and/or Cortical-basal ganglionic degeneration), tauopathies, synucleinopathies, Dementia with Lewy bodies, Dystonia, Cerebral Palsy, Bradykinesia, Chorea, Huntington's Disease, Ataxia, Tremor, Essential Tremor, Myoclonus, tics, Tourette Syndrome, Restless Leg Syndrome, Stiff Person Syndrome, arthritic disorders, stroke, neurodegenerative disorders, upper motor neuron disorders, lower motor neuron disorders, muscle disorders, pain disorders, Multiple Sclerosis, Amyotrophic Lateral Sclerosis, Spinal Cord Injury, Traumatic Brain Injury, Spasticity, Chronic Pain Syndrome, Phantom Limb Pain, Pain Disorders, Metabolic Disorders, and/or traumatic injuries.

Another aspect of the disclosure includes methods for assessing a subject for a movement disorder. Those methods involve receiving a first set of motion data from an image capture device related to at least one joint of a subject while the subject is performing a task, receiving a second set of motion data from an external body motion sensor related to the at least one joint of the subject while the subject is performing the task, calculating, using a computer, kinematic and/or kinetic information about the at least one joint of a subject from a combination of the first and second sets of motion data, and assessing the subject for a movement disorder based on the kinematic and/or kinetic information.

Methods of the disclosure can additionally include receiving balance data of the subject from a force plate, calculating, using a computer, kinematic and/or kinetic information about the at least one joint of a subject from a combination of the first and second sets of motion data and the balance data, and assessing the subject for a movement disorder based on the kinematic and/or kinetic information. The methods can further involve receiving eye movement data, and/or receiving voice data, which both can be used in the calculation of the kinematic and/or kinetic information or complement/augment the kinematic and kinetic data.

Systems and methods of the disclosure can be used in de-novo assessment of a patient for a movement disorder or progression of a movement disorder. Alternatively, systems and methods of the disclosure can be combined with a stimulation protocol and/or a drug protocol to determine how a subject responds to stimulation. In such embodiments, systems of the disclosure may involve stimulation apparatuses and methods of the disclosure may involve providing stimulation to the neural tissue of the subject. The method may be repeated after the subject has received stimulation of their neural tissue, thereby monitoring how a patient has responded to the stimulation they received. That information allows for tuning of subsequent stimulation to better treat the subject.

Since the disclosure includes new systems for analyzing a subject for a movement disorder, aspects of the disclosure also provide new methods for assessing whether a subject is afflicted with a movement disorder. For example, another aspect of the disclosure includes methods of assessing a movement disorder in a subject that involve obtaining a velocity measurement of a joint of a subject while the subject is performing a task, and assessing a movement disorder based on the obtained velocity measurement. Another aspect of the disclosure includes methods of assessing a movement disorder in a subject that involve obtaining a balance characteristic measurement of a subject using a force plate and an external body motion sensor (e.g., an accelerometer) mounted to the subject while the subject is performing a task, and assessing a movement disorder based on the obtained balance characteristic measurement.

Methods and systems associated with the disclosure provide for a motion analysis suite and methods that can aid care providers in motor symptom assessments, differential diagnosis, disease diagnosis, patient/disease classification, prediction of patient recovery, prediction or assessment of disease progression, prediction or assessment of treatment outcome, guiding treatment decisions (e.g., type, course (e.g., dose, duration, delivery timing)), treatment tuning or optimization, prognosis, prediction of new symptoms development, prediction of hospitalization risk, prediction of needed level of assistance, diet optimization, and/or dosing of a treatment. Methods and systems associated with the disclosure provide for a motion analysis suite and methods that can aid providers or patients in the prediction of new symptoms development, prediction of bone fractures risk, prediction of hospitalization risk, prediction of needed level of assistance, and methodologies to assess the effect of different diets/food intakes on motor symptoms.

In a particular embodiment, the system could integrate sensors such as, but not limited to motion capture camera(s), force sensor(s), inertial sensor(s) (e.g., accelerometer /d/ gyroscope), and/or event trigger device(s) to objectively and quantitatively measure patient movement. The sensors can be for example fixed in place, be portable, be mobile, placed on a patient, placed on a care giver (e.g., person conducting assessment), be part of an external device that the patient or caregiver carries with them (e.g., accelerometers in a cell phone), and/or placed in a wearable item. The sensors can be integrated and synchronized via a computing device or computing devices (e.g., integrated chip-based device, computer, tablet, cell phone). The sensors can be connected via wires, wirelessly, or via a memory disk that can be transferred between a sensor and an external computing device and/or between sensors. The patient movement data can be transferred real-time (as its being recorded) or after patient assessments are taken and then transferred to an external system for storage and/or analysis. The computing device(s) can be an external device(s) and/or part of a sensor or sensors. The computing device(s) could control the sensors and synchronize and/or integrate the various sensor information that is recorded from the patient and/or care provider.

The system can include software to derive quantitative movement kinematic/kinetic-based motor evaluations; computational, statistical and/or machine learning algorithms for data reduction, data modeling, predictions of clinical scales and/or prognostic potential for disease recovery, predictions of clinical scales, prediction of response to therapy, guidance of therapy to a particular response, and/or tuning of therapy to particular response. The computational system(s) can be integrated with a database of patient clinical and/or demographic data which could for example be used as part of the statistical and/or machine learning calculations and assessments (additional types of databases, analyses methods, and data types which can be integrated with the method can be found in THE EVOLUTION OF BIG DATA IN NEUROSCIENCE AND NEUROLOGY incorporated hereinabove. The system itself can be designed such that the statistical and/or machine learning calculations and assessments can continually improve their capability (e.g., accuracy of predictions, resolution of assessments) based on the access to database(s) of patient clinical and/or demographic data and/or past calculation and assessment results. In certain embodiments, the system can further be integrated directly with a patient billing and reimbursement database(s) to see that its use or the use of other therapies are properly compensated. In certain embodiments, the system can further be integrated directly with a patient database(s) and/or used to regulate use of the system and/or other therapy. The system itself can be employed directly in person and/or remotely such as via telehealth-based assessments.

The disclosure includes methods that identify biomechanical correlates of symptoms of a movement disorder (in some cases, symptoms not normally captured by the classical clinical scales), and can use such data to tailor therapies based on specific patient biomechanical patterns, such as for example in teaching patients specific compensatory movements based on disease patterns and/or providing brain stimulation therapies focused on specific movement patterns or providing, controlling, or dosing a therapy based on specific patterns recorded during a motor exam conducted with the motion analysis suite.

Aspects of the disclosure make use of the motion analysis system diagnostic tool (motion analysis system as described, for example, in PCT application no. PCT/US14/64814, the content of which is incorporated by reference herein in its entirety) for quantitative and objective assessment of motor symptoms in Parkinson's Disease (PD), stroke, or any such pathology that affects human movement. In certain embodiments the motion analysis suite employs a toolbox of computational methods (e.g., statistical algorithms, machine learning algorithms, optimization methods) such as for example to assess patient movement kinematics and kinetics; reduce data dimensionality; classify patient disease characteristics; highlight patient symptomology; identify patient risk characteristics; model and/or predict disease progression; model and/or predict the response to treatment; and/or tailor a patients treatment course. In certain embodiments the motion analysis suite employs a toolbox of computational methods (e.g., statistical algorithms, machine learning algorithms, optimization methods) such as for example predict new symptoms development, predict bone fracture risk, predict hospitalization risk, predict needed level of assistance, and assess the effect of different diets/food intakes on motor symptoms. For example, the motion analysis suite could employ statistical and machine learning algorithms, based on the patient motion data that is recorded during an exam to improve patient diagnoses and evaluation. For example, early diagnosis of PD is quite challenging, and approximately 20% of new patients go mis-or un-diagnosed; and the motion analysis suite can be used in the differential diagnosis of PD and assist the care giver in making a proper disease diagnosis. Furthermore, disease progression is tracked using coarse clinical scales, such as the Unified Parkinson's Disease Rating Scale (UPDRS), which suffer from limited resolution and high intra-and inter-rater variability; the motion analysis suite could address these limitations, where the motion analysis system is used to measure PD motor symptoms, quantify disease severity, and facilitate diagnosis (such as through statistical algorithms and/or machine learning algorithms). The system may include a battery of portable and/or wearable sensors (including a 3D motion capture video camera (classic RGB and infrared depth-based imaging), inertial sensors, force sensors, and a force plate), which can be used for monitoring and quantifying subjects' motor performance during assessments, such as a UPDRS III focused motor exam. Quantitative metrics can be derived from the motion analysis system recordings to measure primary motor symptoms (e.g., bradykinesia, rigidity, tremor, postural instability). The data from the motion analysis system can be used to build statistical models to extract a low dimensional representation of disease state and to predict disease severity (e.g., UPDRS3). Kinematic/kinetic data not classically captured with clinical scales, such as the UPDRS3 can be identified, including joint kinematics of position, movement trajectory, and movement quality across the motor system, to build full body models of disease state. The computational models can predict response to therapy based on motion analysis suite data by comparing motion analysis suite measures of patients in different states of therapy (such as in their ‘On’ and “Off’ states (i.e., on or off levodopa) or in different states of Deep Brain Stimulation (DBS) (e.g., different stimulation pulse frequencies) for Parkinson's patients), or based on a database of past treated patients and their response to various therapies (e.g., DBS for Parkinson's patients). The entire computational package of the motion analysis suite, including the kinematic/kinetic analysis software, can be combined in a patient-tracking database, capable of providing motion analysis system data that enhances classical clinical information (e.g., classical clinical UPDRS information). Additionally, in certain embodiments other clinical data can be combined with the motion analysis system and its prediction/AI component can be used to assess and predict the risk of bone fractures and/or fall risk.

The motion analysis suite could employ statistical and machine learning algorithms, based on the patient motion data that is recorded during an exam to improve patient prognosis. For example, prediction of recovery from stroke can be quite challenging; and the motion analysis suite can be employed to predict the likelihood of the patient recovering from stroke in the acute setting. By integrating the different proposed individual sensor types, the system can for example first assist in performing more accurate, less variable motor exams, and symptom assessments with higher resolutions than classic clinical scales as the sensors can be used to objectively track and measure patient movements without subjective limitations of typical clinical assessments (Functional Assessment for Acute Stroke Trials: Properties, Analysis, and Application, Taylor-Rowan, 2018, DOI: 10.3389/fneur.2018.00191). Furthermore, stroke is a multi-symptom disease of varied, yet often correlated symptoms, which is necessarily described in a “probabilistic” manner, especially when predicting motor recovery (Functional potential in chronic stroke patients depends on corticospinal tract integrity,” DOI: 10.1093/brain/aw1333, https://www.ncbi.nlm.nih.gov/pubmed/17148468). Machine learning algorithms can be implemented to generate predictions of clinical scales (such as the Fugl Meyer Stroke scale, or the NIH Stroke Scale); predictions of motor recovery based on integrated symptom assessment; and/or patient classification based on well-studied statistical algorithms (e.g., sensor-based kinematics data can be collected during assessments with the motion analysis suite along and combined with data from past exams and/or data derived from typical patient characteristics to build a generalized linear model which predicts a patients stroke scale scores or likelihood of recovery based on the motion analysis data input (and or other clinical information collected from the patient)). As the motion analysis suite could make use of data collected from a single joint or across multiple joints throughout the body, the system allows for the development of both single joint and full body models of disease impact on movement. The computational approach with the motion analysis suite can build upon the integration of sensors that provides for a synchronized data acquisition of patient kinematics and the statistical algorithms can be employed to computationally analyze the stroke injury state, through data dimensionality reduction and prediction methods to provide the clinician with a tool to aid and augment the classic evaluation process.

In another embodiment, when integrated with other patient clinical data and/or data provided by the patient regarding their habits the integrated sensor-based motion analysis suite concepts can be used for prediction of new symptoms development, prediction of bone fractures risk, prediction of hospitalization risk, and prediction of needed level of assistance. The integrated sensor-based motion analysis suite concepts can also be used to assess the effects of different diets/food intakes on motor symptoms. For example, different foods can interfere with levodopa and reduce its effectiveness, leading to the necessity of using higher doses. The suite can be used to tabulate/track these effects and help the patient or their provider to optimize their diet and optimal dosage of medication.

Additional ways in which the integrated sensor-based motion analysis suite concepts can be implemented include as a training tool to teach someone to perform an exam, or the motion analysis suite can be designed so that the identification and/or classification routines use machine learning techniques to improve or tune to a particular user, users, or standards. In certain embodiments the system can further be used to aid in the design of tools or environments for assisting people in the activities of daily living and/or in assisting people in avoiding falls. Such as for example, a motion analysis system can be implemented in a patient's home environment and used to observe the patient's activities and/or movements in or through their home environment. The analysis of the patient's movement patterns can be used to identify activities and/or locations in their home environment that are associated with a risk of falling and/or performed sub-optimally and be used to design a safer home environment for the patient (e.g., identify a floor plan, furniture, activities that elevate risk) and/or used to identify and train patients how to improve their movements. The system could also, for example, be used to train and optimize a person or a team of people to perform an activity, such as training for a sporting event or for a mission to be completed by the military or first responders. Further exemplary therapies, training techniques, and/or activities that can be integrated with or improved via the device embodiments and methods discussed herein could include physical therapy, occupational therapy, chiropractic therapy, cognitive therapy, behavioral therapy, cognitive-behavioral therapy, Mentalization-based therapy, drug therapy, brain stimulation therapy, robot assisted therapy, psychotherapy, rehabilitation therapy, mindfulness therapy, bio-feedback based therapy, surgical interventions, Eye movement desensitization and reprocessing therapy, augmentation therapy and/or training, physical fitness activities and/or training such as weight or strength training, mobility training, balance training, flexibility training, cardiovascular training, mirror therapy, martial arts training, yoga (single or partner based), dance, psychophysics assessments and/or observations, video game design and/or implementation, virtual reality based treatments, virtual reality, virtual reality based training, augmented reality therapy, augmented reality, augmented reality based training, robot training, robot control, training robot control, animation design such as used in the entertainment industry, prosthetic therapy and/or design, and/or any such combination. Aspects of the disclosure make use of the motion analysis system and its prediction/AI component for measuring motor performance of a subject in select motor task and use the prediction/AI component for predicting the performance of the subject in sports. For example, the motion analysis system can be used for measuring the subject's balance under select conditions (e.g., stable terrain, rough terrain, wearing specific garments or shoes) for predicting performance of the subject in a competition in a sport that particularly requires balance (e.g., dancing, ice-skating). Such a system could also be used for classifying athletes or for devising programs for training athletes. Additionally, aspects of the disclosure make use of the motion analysis system and its prediction/AI component for measuring motor performance of a subject in select motor tasks and use the prediction/AI component for predicting the subject's likelihood to get injured. For example, the motion analysis system can be used for measuring the subject's balance under select conditions (e.g., stable terrain, rough terrain) for predicting likelihood that the subject will get injured in a sport, job, or task that particularly requires balance. Furthermore, aspects of the disclosure make use of the motion analysis system and its prediction/AI component for measuring motor performance of a surgeon in select motor tasks and use the prediction/AI component for predicting the surgeon's performance. For example, the motion analysis system can be used for measuring the surgeon's motor skills/dexterity during certain precision tasks (e.g., time to complete a surgical maneuver, hand joints movement smoothness) and for predicting performance of the surgeon in a class of surgeries that require that specific set of skills. Such a system could also be used for classifying surgeons or for devising programs for training surgeons. Additionally, aspects of the disclosure make use of the motion analysis system and its prediction/AI component for measuring balance in subjects potentially prone to falls in select conditions ((e.g., stable terrain, rough terrain) and use the prediction/AI component for predicting the fall risk of the patient in everyday life. Such a system could also be used for classifying subjects based on fall risks or devising training programs for such subjects. The system could in turn be used to identify a subject that needs a caregiver and match the patient with the appropriate caregiver.

Additional embodiments of the device allow for use with brain stimulation and/or neuromodulation devices, biophysical dosing software, motion analysis suite(s), cost effective analysis software, big data and/or big data analysis methods, diagnostics, prognostics, health care and/or combined elements (e.g., Big Data Application of a Personalized Therapy Suite and the Associated Elements) such as, for example, with a motion analysis suite or suites and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment. Certain embodiments allow for the use of the above methods with or without the motion analysis suite(s).

Any type of stimulation known in the art may be used with methods of the disclosure, and the stimulation may be provided in any clinically acceptable manner. For example, the stimulation may be provided invasively or noninvasively. Preferably, the stimulation is provided in a noninvasive manner. For example, electrodes may be configured to be applied to the specified tissue, tissues, or adjacent tissues. As one alternative, the electric source may be implanted inside the specified tissue, tissues, or adjacent tissues.

Exemplary types of stimulation include chemical, mechanical, thermal, optical, electromagnetic, thermal, or combinations thereof. In particular embodiments, the stimulation is a mechanical field (i.e., acoustic field), such as that produced by an ultrasound device. In other embodiments, the stimulation is an electrical field. In other embodiments, the stimulation is a magnetic field. Other exemplary types of stimulation include Transcranial Direct Current Stimulation (TDCS), Transcranial Ultrasound (TUS), Transcranial Doppler Ultrasound (TDUS), Transcranial Electrical Stimulation (TES), Transcranial Alternating Current Stimulation (TACS), Cranial Electrical Stimulation (CES), Transcranial Magnetic Stimulation (TMS), temporal interference, optical stimulation, Infrared stimulation, near infrared stimulation, optogenetic stimulation, nanomaterial enabled stimulation, thermal based stimulation, chemical based stimulation, and/or combined methods. Other exemplary types include implant methods such as deep brain stimulation (DBS), micro-stimulation, spinal cord stimulation (SCS), and vagal nerve stimulation (VNS). Other exemplary forms of stimulation include sensory stimulation such as multi-gamma stimulation.

In other embodiments, the stimulation is provided by a combination of an electric field and a mechanical field. The electric field may be pulsed, time varying, pulsed a plurality of time with each pulse being for a different length of time, or time invariant. Generally, the electric source is current that has a frequency from about DC to approximately 100,000 Hz. The mechanical field may be pulsed, time varying, or pulsed a plurality of time with each pulse being for a different length of time. In certain embodiments, the electric field is a DC electric field.

The stimulation may be applied to a structure or multiple structures within the brain or the nervous system. Exemplary structures include dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord.

In one exemplary embodiment, the electric field is applied broadly, and mechanical field is focused on a specific brain structure or multiple structures for therapeutic purposes. The electric field may be applied broadly and the mechanical field may be focused on a structure or multiple structures, such as brain or nervous tissues including dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and/or spinal cord. Other possible configurations include applying both the electrical field and the mechanical field in a broad manner; applying both the electric field and the mechanical field in a focused manner; or applying the electric field in a focused manner and the mechanical field in a broad manner.

Furthermore, the disclosure includes methods to account for stimulation fields (e.g., based on tissue filtering data) that can be used to predict a tissue's response to stimulation, and thus methods of the disclosure are useful for optimizing stimulation waveforms used in clinical stimulators for a programmed stimulation effect on tissue. Methods of the disclosure predict stimulation electromagnetic field distribution information including location (target), area and/or volume, magnitude, timing, phase, frequency, and/or direction and also importantly integrate with membrane, cellular, tissue, network, organ, and organism models.

In certain aspects, the disclosure includes methods for stimulating tissue that involve analyzing at least one filtering property of a region of at least one tissue, and providing a dose of energy to the at least one region of tissue based upon results of the analyzing step. Exemplary filtering properties include anatomy of the tissue (e.g., distribution and location), electromagnetic properties of the tissue, cellular distribution in the tissue, chemical properties of the tissue, mechanical properties of the tissue, thermodynamic properties of the tissue, chemical distributions in the tissue, and/or optical properties of the tissue. Methods of the disclosure can be implemented during stimulation, after stimulation, or before stimulation (such as where dosing and filtering analysis could take place via simulation).

Any type of energy known in the art may be used with methods of the disclosure. In certain embodiments, the type of energy is mechanical energy (which includes sonic (a.k.a. acoustic) energy), such as that produced by an ultrasound device. In certain embodiments, the ultrasound device includes a focusing element so that the mechanical field may be focused. In other embodiments, the mechanical energy is combined with an additional type of energy, such as chemical, optical, electromagnetic, or thermal energy.

In other embodiments, the type of energy is electrical energy, such as that produced by placing at least one electrode in or near the tissue. In certain embodiments, the electrical energy is focused, and focusing may be accomplished based upon placement of electrodes. In other embodiments, the electrical energy is combined with an additional type of energy, such as mechanical, chemical, optical, electromagnetic, or thermal energy.

In particular embodiments, the energy is a combination of an electric field and a mechanical field. The electric field may be pulsed, time varying, pulsed a plurality of time with each pulse being for a different length of time, or time invariant. The mechanical filed may be pulsed, time varying, or pulsed a plurality of time with each pulse being for a different length of time. In certain embodiments, the electric field and/or the mechanical field is focused.

The energy may be applied to any tissue. In certain embodiments, the energy is applied to a structure or multiple structures within the brain or the nervous system such as the dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord. In particular embodiments, the tissue is neural tissue, and the effect of the stimulation alters neural function past the duration of stimulation.

Another aspect of the disclosure includes methods for stimulating tissue that involve providing a dose of energy to a region of tissue in which the dose provided is based upon at least one filtering property of the region of tissue. Another aspect of the disclosure includes methods for stimulating tissue that involve analyzing at least one filtering property of a region of tissue, providing a dose of electrical energy to the region of tissue, and providing a dose of mechanical energy to the region of tissue, wherein the combined dose of energy provided to the tissue is based upon results of the analyzing step. Another aspect of the disclosure includes methods for stimulating tissue that involve providing a noninvasive transcranial neural stimulator and using the stimulator to stimulate a region of tissue, wherein a dose of energy provided to the region of tissue is based upon at least one filtering property of the region of tissue.

The focused tissue may be selected such that a wide variety of pathologies may be treated. Such pathologies that may be treated include but are not limited to Multiple Sclerosis, Amyotrophic Lateral Sclerosis, Alzheimer's Disease, Tics, Parkinson's Disease, Huntington's Disease, Muscular Dystrophy, Cerebral Palsy, Stroke, Myasthenia Gravis, Peripheral Neuropathy, Ataxia, Friedreich's Ataxia, Dystonia, Restless Leg Syndrome, Polio (Poliomyelitis), Guillain-Barré Syndrome, Post-Polio Syndrome, Rheumatoid Arthritis, Osteoarthritis, Lupus, Tardive Dyskinesia, Chorea, Hemiballismus, Wilson's Disease, Brachial Plexus Injury, Tetanus, Motor Neuron Disease, Bell's Palsy, Essential Tremor, Orthostatic Tremor, Rett Syndrome, Spinocerebellar Ataxia, Spinal Muscular Atrophy, Primary Lateral Sclerosis (PLS), Charcot-Marie-Tooth Disease, Complex Regional Pain Syndrome (CRPS), Fibromyalgia, Progressive Supranuclear Palsy, Myoclonus, Phantom Limb Pain, Syringomyelia, Trigeminal Neuralgia, Osteoporosis, Ankylosing Spondylitis, Gout, Paget's Disease of Bone, Lyme Disease, Botulism, Tourette's Syndrome, Prion Diseases, Creutzfeldt-Jakob Disease, Stiff Person Syndrome (SPS), Dermatomyositis, Scleroderma, Batten Disease, Narcolepsy, Chronic Fatigue Syndrome (CFS), Machado-Joseph Disease, Benign Essential Blepharospasm, Foot Drop, Carpal Tunnel Syndrome, Peripheral Artery Disease, Reflex Sympathetic Dystrophy Syndrome, Pantothenate Kinase-Associated Neurodegeneration (PKAN), Mitochondrial Myopathies, Paraneoplastic Syndromes of the Nervous System, Chronic Inflammatory Demyelinating Polyneuropathy (CIDP), Progressive Multifocal Leukoencephalopathy, Transverse Myelitis, Myotonic Dystrophy, Cervical Spondylosis, Behçet's Disease, Pseudotumor Cerebri, Krabbe Disease, Neurofibromatosis, Acoustic Neuroma, Vestibular Neuritis and Labyrinthitis, Vertigo, Meniere's Disease, Chronic Paroxysmal Hemicrania, Antiphospholipid Syndrome (APS), Neuralgia, Paralysis, Postural Orthostatic Tachycardia Syndrome (POTS), Shy-Drager Syndrome, Vasculitis, Hemifacial Spasm, Isaacs' Syndrome, Marfan Syndrome, Osteogenesis Imperfecta, Ehlers-Danlos Syndromes, Alkaptonuria, Spasticity, Athetosis, Hyperkinesias, Hypokinesias, Meralgia Paresthetica, Restless Arms Syndrome, Piriformis Syndrome Spinal Cord Injury, Traumatic Brain Injury, Drug Craving, Food Craving, Alcohol Craving, Nicotine Craving, Craving, Addiction, Drug Abuse, Brain Injury, Diabetes, Cardiovascular Condition, Pulmonary Condition, Balance Ailment, Impingement Syndromes, Joint Replacement, Bone Fusion, bone fracture, joint injury, Trauma, Peripheral Nerve Injury, Post Surgery Injury, Declined Motor Performance, Stuttering, Tinnitus, Spasticity, Parkinsonianism, Obsessions, Depression, Schizophrenia, Bipolar Disorder, Acute Mania, Catatonia, Post-Traumatic Stress Disorder, Stroke, Cognitive Decline, Motor Dysfunction, Motor Performance Decline, Autism, Chronic Pain Syndrome, Epilepsy, Stroke, Auditory Hallucinations, Movement Disorders, Neurodegenerative Disorders, Pain Disorders, Metabolic Disorders, Addictive Disorders, Psychiatric Disorders, Traumatic Nerve Injury, and/or Sensory Disorders. Furthermore, stimulation may be focused on specific brain or neural structures to enact procedures including sensory augmentation, sensory alteration, anesthesia induction and maintenance, brain mapping, epileptic mapping, neural atrophy reduction, neuroprosthetic interaction or control with nervous system, stroke and traumatic injury neurorehabilitation, bladder control, vestibular stimulation, locomotion augmentation, movement augmentation, assisting breathing, cardiac pacing, muscle stimulation, and treatment of pain syndromes, such as those caused by migraine, neuropathies, and low-back pain; or internal visceral diseases, such as chronic pancreatitis or cancer. The methods herein could be expanded to any form of arthritis, impingement disorders, overuse injuries, entrapment disorders, and/or any muscle, skeletal, or connective tissue disorder which leads to chronic pain, central sensitization of the pain signals, and/or an inflammatory response.

In yet another embodiment, the method according to the present disclosure with stimulation can be applied the area of physical therapy, where amplified, focused, direction altered, and/or attenuated currents could be used to stimulate blood flow, increase or alter neuromuscular response, limit inflammation, speed the breakdown of scar tissue, and speed rehabilitation by applying the focus of the current generation to the effected region in need of physical therapy. It is envisioned that the method according to the present disclosure may have a wide variety in the area of physical therapy including the treatment or rehabilitation of traumatic injuries, sports injuries, surgical rehabilitation, occupational therapy, and assisted rehabilitation following neural or muscular injury. For instance, following an injury to a joint or muscle, there is often increased inflammation and scar tissue in the region and decreased neural and muscular response. Typically, ultrasound is provided to the affected region to increase blood flow to the region and increase the metabolic re-absorption of the scar tissue while electrical stimulation is provided separately to the nerves and muscles; however, by providing them together, a person could receive the benefit of each individual effect, but additionally amplified stimulatory and metabolic effects through the altered currents. The other methods for generating altered currents discussed within could also be used to assist in physical therapy via the displacement currents that are generated. It should be noted that this idea can be implemented independent of stimulation and just as part of the motion analysis suite(s) and vice versa.

Furthermore, another embodiment incorporates the use of big data and big data methods (including additional types of big data databases, big data analyses methods, and big data types which can be integrated with the method can be found in THE EVOLUTION OF BIG DATA IN NEUROSCIENCE AND NEUROLOGY incorporated hereinabove alone or with the motion analysis, brain stimulation, and/or other devices or methods disclosed herein. As an additional embodiment we present a way to acquire large real-time multimodal data sets such as for use in personalized care in the movement disorder, pain, and rehabilitation spaces with an Integrated Motion Analysis Suite (IMAS), which combines motion capture camera(s), inertial sensors (gyroscope/accelerometers), and force sensors to assess patient movement kinematics from joint(s) across the body and kinetics. The technology can holistically aid clinicians in motor symptom assessments, patient classification, and/or prediction of recovery or response to treatment. The hardware system for movement kinematic and kinetic data capture is underpinned with an artificial intelligence (AI) driven computational system with algorithms for data reduction, modeling, and predictions of clinical scales and prognostic potential for motor recovery (or response to treatment).

In some embodiments, the AI driven computational system involves a trained machine learning model comprising an artificial neural network including a number of input nodes, one or more hidden layers, and a number of output nodes, wherein each input node includes a memory location for storing input values including raw image data from the data captures. In some embodiments, the trained machine learning model is also configured to generate a number of risk scores corresponding to the one or more patient movement characteristics. The systems and methods described herein may rely on a machine learning model configured to identify movement anomalies that are not visible to thew naked eye based on the collected data and machine learning, which may encompass artificial intelligence and deep learning concepts, such as, for example, the use of classic neural networks. Additionally, the image data collected herein may refer to a combination of multiple images from various angles, ambient conditions, wavelengths, etc. and may be different from what a human can see in person. This means that certain features may not be strictly “visible patterns,” but instead can be patterns or trends found in numbers (aka “numerical patterns”) that have been determined by an AI algorithm (e.g., a computer can convert image data to numerical data during processing). These features can be selected manually or by using an AI model using various deep learning architectures in a supervised, unsupervised, or semi-supervised manner. In one embodiment, features can be selected using an AI model by directly feeding post-processed or raw images into a model architecture. Similarly, language data, sound data, text data, biospecimen data, biophysical data, etc. can be different from what a person could perceive and can be assessed/processed via a similar pattern.

Numerous statistical and/or AI methods can be employed such as regression modeling, generalized linear modeling, generalized nonlinear modeling, least absolute shrinkage and selection operator (LASSO), LASSO or elastic net regularization for linear models, linear support vector machine models, Empirical risk minimization (ERM), neural network learning, such as those are exemplified in (Applied Predictive Modeling, M. Kuhn, K. Johnson (Author), 2018, Springer; Handbook of Deep Learning in Biomedical Engineering), V. E. Balas, B. K. Mishra, R. Kumar, 2021, Academic Press; Statistical and Machine Learning Data Mining, B. Ratner, 2011, CRC Press) the content of each of which is incorporated by reference herein in their entirety. The model(s) of prediction and/or inference can further be optimized via additional machine /arning/ artificial intelligence (AI) methods such as deep learning. Methods used herein could for example be selected from examples such as: Supervised Learning; Unsupervised Learning; Reinforcement Learning; Semi-Supervised Learning; Deep Learning (e.g., Convolutional Neural Networks, Recurrent Neural Networks); Neural Networks; Decision Trees (e.g., ID3, CART); Random Forests; Gradient Boosting Machines (e.g., XGBoost, LightGBM); Support Vector Machines (SVM); Regression (e.g., Linear, Polynomial, Logistic); Naive Bayes; K-Means Clustering; Hierarchical Clustering; DBSCAN; Anomaly Detection; Principal Component Analysis (PCA); Linear Discriminant Analysis (LDA); Ensemble Learning (e.g., Bagging, Boosting); Cross-Validation; Regularization (e.g., L1, L2); Transfer Learning; Neural Architecture Search; Genetic Algorithms; Bayesian Networks; Hidden Markov Models; Long Short-Term Memory (LSTM); Gated Recurrent Units (GRU); Attention Mechanisms; Transformer Architectures (e.g., BERT, GPT-2/3); Reinforcement Learning Algorithms (e.g., Q-learning, Deep Q Learning, Monte Carlo methods); Simulated Annealing; Boltzmann Machines; Radial Basis Function Networks (RBFN); Self-Organizing Maps; Optimization Algorithms (e.g., Gradient Descent, Adam, RMSprop); Swarm Intelligence (e.g., Particle Swarm Optimization, Ant Colony Optimization); Fuzzy Systems; Expert Systems; Nearest Neighbors (e.g., k-NN); Sequence Mining; Rule-Based Systems; Affinity Analysis; Neural Turing Machines; Capsule Networks; AutoML; Adversarial Networks (e.g., GANs); Time Series Forecasting; Natural Language Processing (NLP); Image and Video Analysis; Speech Recognition; Chatbots and Conversational AI; Robotic Process Automation (RPA); Facial Recognition; Sentiment Analysis; Recommendation Systems; Optical Character Recognition (OCR); Regression Analysis (e.g., Ridge, Lasso, ElasticNet); Feature Engineering; Feature Selection; Dimensionality Reduction; Bias-Variance Tradeoff; One-Hot Encoding; Tokenization in NLP; Word Embeddings (e.g., Word2Vec, GloVe); Named Entity Recognition (NER); Machine Translation; Q-Learning and SARSA in Reinforcement Learning; Thompson Sampling and UCB in Multi-Armed Bandits; Data Augmentation; Dropout in Neural Networks; Backpropagation; Convolutions in CNNs; Sequence Models for Time Series and Forecasting; Multi-Agent Systems; Collaborative Filtering; Content-Based Filtering; Multilayer Perceptrons (MLP); Synthetic Data Generation; Relational Databases and SQL for Machine Learning; Evolutionary Computation; Generative Adversarial Networks (GANs); Variational Autoencoders (VAEs); Normalization Techniques (e.g., Batch Normalization, Group Normalization); Few-Shot Learning; Information Retrieval; Data Imputation; Recursive Neural Networks; Data Labeling and Annotation; Active Learning; Continual Learning; Multimodal Learning; Causal Inference; Anomaly and Outlier Detection; Emotion AI or Affective Computing; Vision Systems (e.g., OpenCV); Audio and Speech Processing Systems; Context-Aware Computing; Multitask Learning; Quantum Computing and Quantum Machine Learning; Sparse Modeling and Regularization Techniques (e.g., LASSO, Ridge Regression, ElasticNet); Kernel Methods (e.g., Kernel SVM, Gaussian Processes); Multi-task Learning Techniques (e.g., Joint Learning, Hard Parameter Sharing); Meta-Learning (e.g., MAML, Prototypical Networks); One-shot and Zero-shot Learning (e.g., Siamese Networks, Triplet Loss); Active Learning (e.g., Uncertainty Sampling, Query-by-Committee); Semi-Supervised Learning Techniques (e.g., Label Propagation, Self-training); Autoencoders; Generative Models; Non-negative Matrix Factorization (NMF); Neural Network Initializations and Activations (e.g., Xavier Initialization, He Initialization, ReLU variants); Entity and Positional Embeddings; Advanced NLP Techniques (e.g., Sequence-to-Sequence with Attention, BPE, Transformer Architectures); Fairness and Interpretability in AI (e.g., LIME, SHAP); Mini-batch Gradient Descent, Online Learning, and Federated Learning; Evolutionary Algorithms (e.g., Evolution Strategies, Genetic Programming); U-Nets, WaveNet; Topic Modeling (e.g., Latent Dirichlet Allocation), Word Sense Disambiguation; Neuro-fuzzy Systems, Neuro-evolutionary Systems; Model-Based Reinforcement Learning, Inverse Reinforcement Learning; Counterfactual Explanations, Activation Maximization; Out-of-core Algorithms; Curriculum Learning; Lifelong Learning; Association Rule Learning; Bayesian Optimization; Contrastive Learning; Self-supervised Learning.

The system has been designed so multiple systems can be networked together and multiple patients' kinematic/kinetic data, imaging, and clinical data can be longitudinally assessed and analyzed to develop a continually improving model of patient recovery (or as a method to personalize and optimize therapy delivery and predicting response to therapy-see below). The system is also designed with the capability to integrate with real-world data (e.g., electronic health records, payer databases) to further power the model. We have also developed a new form of noninvasive brain stimulation, electrosonic stimulation (ESStim). The system(s) allow for assessment of stimulation efficacy through combined imaging data, clinical data, biospecimen data, kinematic data, and/or patient specific biophysical models of stimulation dose at the targeted brain sites to identify best responders to therapy (e.g., in PD, OUD, and Pain). The system(s) supports computational models to identify the best responders to therapy and/or as a means to personalize therapy based on the unique characteristics of the individual patients. The IMAS system, with its big data backbone, can be integrated with the ESStim system (or any type of brain stimulation and/or treatment method) to further aid in personalizing patient stimulation dose in certain indications (e.g., Parkinson's Disease, Chronic Knee Pain). We can also integrate this system with a trial optimization tool based on health economics. The software allows for a virtual trial design and predicting the trials cost effectiveness. Furthermore, the software can be implemented as a means to quantify data set values such as to quantitively support decision maker policy. Ultimately, the systems can be combined to allow for the use in a personalized treatment suite, based on a big data infrastructure, whereby the multimodal data sets (e.g., imaging, biophysical field-tissue interaction models, clinical, and biospecimen data) are coupled rapidly to personalize brain stimulation-based treatments in a diverse and expansive patient cohorts.

Another embodiment implements big data approaches to optimize therapy by combining connectome information with the motion analysis system(s) and/or brain stimulation treatment methods. This method can be used to optimize brain stimulation doses or other forms of therapy (e.g., physical therapy). Another embodiment implements big data imaging methods to optimize therapy with the motion analysis system(s) and/or brain stimulation treatment methods. Furthermore, another embodiment implements big data genetics methods to optimize therapy with the motion analysis system(s) and/or brain stimulation treatment methods. See, for example, U.S. pat. publ. no. 2011/0245734, the disclosure of which is hereby incorporated herein in its entirety.

Furthermore, another embodiment includes the use of Health Economics methods including software and computational based methods for determining an optimized design or cost-effective design for a clinical trial, such as a Randomized Controlled Trial (RCT) for evaluating medical therapies, and/or methods for optimizing a patient's therapy.

Furthermore, another embodiment includes the use of Health Economics methods including software and computational based methods for determining an optimized design or cost-effective design for a clinical trial, such as a Randomized Controlled Trial (RCT) for evaluating medical therapies, and/or methods for optimizing a patient's therapy.

In another embodiment the motion analysis suite is used to assess patient motor abilities and/or this data is matched with specific physical therapy exercises that are provided to the patient in the form of videos or other instructions (e.g., verbal written, graphical). For example, if the suite and its algorithms and/or a diagnosis from another care provider find that the patient movement is bradykinetic the video provided to the patient shows motor exercises aimed at improving movement speed; if the suite and its algorithms finds that a patient joint is rigid the video provided to the patient shows motor exercises aimed at improving rigidity. One or more videos can be provided to the patient. The videos can be selected in multiple ways, including manually, using a look-up table, and/or an algorithm (e.g., an algorithm that determines the optimal length and type of exercise while respecting constraints set by the user (e.g., prioritizing some exercises/physical therapy goals or keeping the session length within a certain time frame)). In this embodiment the motion analysis suite can be used to periodically assess the patient progress and its data and algorithms can be used to devise more (or less) challenging physical therapy exercises based on patient achievements.

In other embodiments the suite and/or its analysis algorithms are used to assess patient motor abilities and or motor learning abilities and/or this data is matched with specific physical therapy exercises to improve motor abilities and/or motor learning abilities. In other embodiment the motion analysis suite and/or its algorithms are used to assess patient motor abilities and/or its data are used to match the patient with specific aids, orthoses, and/or footwear. In other embodiments the suite and/or its algorithms are used in conjunction with a videogame system where the videogame is designed to train/exercise specific movements. In other embodiments the suite and/or its algorithms can be integrated with a model or use a model such as a Natural Language Processing model and/or with a Large Language Model such as to facilitate communication and/or automate processes taking place with the system(s).

In another embodiment the system(s) discussed herein and/or its algorithm(s) can be integrated with a model for Generative Artificial Intelligence (AI) such as to facilitate communication (e.g., AI trained on items such as text, code, images, music, and/or video and/or AI used to provide outputs such as text, code, images, music, and/or video), provide a provide visual communications or figures such as for aiding in explaining activities, provide molecular data information (e.g., AI trained on molecular data such as part of biospecimen(s) and/or AI used to provide outputs of molecular data such as part of biospecimen(s)), provide movement information whereby the generative AI is trained on patient movements to generate output trajectories of new movements such as could be used for therapy (e.g., physical therapy, occupational therapy, sports therapy, and/or to optimize athletic training), provide verbal and/or sound information, and/or automate processes taking place with the system(s).

Furthermore, another embodiment includes the implementation of big data and big data approaches to the brain stimulation and/or neuromodulation devices, biophysical dosing software, motion analysis suite(s), cost effective analysis software, diagnostics, prognostics, health care and/or combined elements.

Another aspect of this disclosure is related to integrating stimulation and/or the motion analysis suite(s) with mechanisms that are used to monitor a patient's response to the stimulation and/or to fine tune the stimulation parameters (e.g., imaging, biofeedback, physiological response) for maximum clinical effect.

Various parameters of the systems and methods disclosed herein will vary to suit a particular application, and the systems and methods disclosed herein may incorporate any of the additional method steps that correspond to the systems herein, and vice-versa. For example, the systems and methods may be used for analyzing other patterns and used in conjunction with providing neurostimulation.
Embodiment 1: A method of determining a management plan for a patient with a disorder (e.g., a movement disorder). The method comprising providing a motion analysis system; obtaining kinematic and/or kinetic information of a patient with a movement disorder using the motion analysis system while the patient is performing a task; determining biomechanical patterns of the patient based on the obtained kinematic and/or kinetic information; and determining a management plan for the patient based on the biomechanical patterns. The methods may include determining other patterns or characteristics of a patient, such as, for example, physiological, movement, postural, etc.
Embodiment 2: A method for assessing a subject. The method comprising obtaining individual kinematic and/or kinetic information of a subject, wherein the kinematic and/or kinetic information of the subject is generated from a motion analysis system; obtaining population kinematic and/or kinetic information from a population of subjectsthat present with similar kinematic and/or kinetic information as that of the subject, wherein thekinematic and/or kinetic information of each member of the population is generated from a motion analysis system; and assessing the subject based on a combination of the individual kinematic and/or kinetic information and the population kinematic and/or kinetic information.
Embodiment 3: A method of determining a management plan for a patient with a movement disorder. The method comprising providing a motion analysis system; obtaining kinematic and/or kinetic information of a patient with a movement disorder using the motion analysis system while the patient is performing a task; and determining a multi-joint or multi-symptom model via computational analysis of the kinematic and/or kinetic information.
Embodiment 4: A system comprised of at least two motion analysis systems connected via a network, wherein the motion analysis systems contain at least one sensing device configured to obtain and transmit at least a set of motion data; at least one synchronization clock; and a central processing unit (CPU) with storage coupled to the CPU for storing instructions that when executed by the CPU cause the CPU to receive the set of motion data from the sensing device related to at least one body part of a subject while the subject is performing a task; and whereby the CPU is further configured to determine a management plan for the patient based on the set of motion data.
Embodiment 5: A system comprised of at least a motion analysis system connected to a central computer, wherein the motion analysis systems contain at least one sensing device configured to obtain and transmit at least a set of motion data; at least one synchronization clock; and wherein the central computer contains a central processing unit (CPU) with storage coupled to the CPU for storing instructions that when executed by the CPU cause the CPU to receive the set of motion data from the motion analysis system; and whereby the CPU is further configured to determine a management plan for the patient based on the set of motion data.
Embodiment 6A: A system for optimizing the design of a clinical trial, the system comprising a computational hardware device, with a software capable of defining a fundamental design goal of effectiveness of the trial, wherein the software is capable of assessing a simulated design of the trial and wherein the design goal of effectiveness of the trial is assessed relative to the simulated design of the trial.
Embodiment 6B: A system for optimizing the treatment of a patient, the system comprising a computational hardware device, with a software capable of defining a fundamental design goal of effectiveness of the treatment, wherein the software is capable of assessing a simulated design of the treatment and wherein the design goal of effectiveness of the treatment is assessed relative to the simulated design of the treatment.
Embodiment 6C: A system for optimizing the treatment of a patient, the system comprising a computational hardware device, with a software capable of defining a fundamental design goal of effectiveness of the treatment, wherein the software is capable of assessing ongoing treatment criteria and wherein the design goal of effectiveness of the treatment is assessed relative to the ongoing treatment criteria.
Embodiment 7A: A method for optimizing the design of a clinical trial. The method comprising defining a fundamental design goal of effectiveness of the trial, wherein the method is capable of assessing a simulated design of the trial and wherein the design goal of effectiveness of the trial is assessed relative to the simulated design of the trial.
Embodiment 7B: A method for optimizing the design of a treatment. The method comprising defining a fundamental design goal of effectiveness of the treatment, wherein the method is capable of assessing a simulated design of the treatment and wherein the design goal of effectiveness of the treatment is assessed relative to the simulated design of the treatment.
Embodiment 7C: A method for optimizing the design of a treatment. The method comprising defining a fundamental design goal of effectiveness of the treatment, wherein the method is capable of assessing an ongoing treatment criteria and wherein the design goal of effectiveness of the treatment is assessed relative to the ongoing treatment criteria.
Embodiment 8: A system for optimizing a treatment of a patient. The system comprising a motion analysis system; an image capture device configured to capture a first set of motion data related to at least one joint of a subject while the subject is performing a task; at least one external body motion sensor configured to capture a second set of motion data related to the at least one joint of the subject while the subject is performing the task; and a computational hardware device, with a software capable of integrating the first and second sets of data received from the image capture device and the external body motion sensor, determining kinematic and/or kinetic information about the at least one joint of the subject from a combination of the first and second sets of motion data, and outputting the kinematic and/or kinetic information of the subject.
Embodiment 9: The system and/or method of any one of Embodiments 1 to 8, or any combination thereof, wherein the management plan comprises at least one of changes to an existing therapy regimen, generation of a new therapy regimen, guidance on physical therapy, guidance on movement types to be performed while the patient is performingan activity, or combinations thereof.
Embodiment 10: The system and/or method of any one of Embodiments 1 to 9, or any combination thereof, further comprising obtaining additional kinematic and/or kinetic information of the patient at a subsequent point in time and updating the management plan based on the additional kinematic and/or kinetic information.
Embodiment 11: The system and/or method of any one of Embodiments 1 to 10, or any combination thereof, further comprising communicating the management plan to the patient.
Embodiment 12: The system and/or method of any one of Embodiments 1 to 11, or any combination thereof, wherein the task is selected from the group consisting of discrete flexion of a joint; discrete extension of a joint; continuous flexion of a joint; continuousextension of a joint; flexion of a joint; extension of a hand; walking; abduction of a joint, adduction of a joint, rotation of a joint, circumduction, pronation, supination, deviation, rotation, stabilizing a joint, reaching, grasping, flexion, extension, abduction, adduction, medial (internal) rotation, lateral (external) rotation, circumduction, pronation, supination, radial deviation (or radial flexion), ulnar deviation (or ulnar flexion), opposition, reposition, dorsiflexion, plantarflexion, inversion, eversion, walking, running, pivoting, leg swing, arm swing, bending, reaching, twisting, sitting to standing, standing, squatting, holding a prone position, holding a static position, lying to sitting, stepping up or down, weight shifting, postural sway, tilting, turning, nodding, pushes or pulls, carrying or lifting, walking on slippery or uneven surfaces, visual challenges, dual-tasking, or combinations thereof.
Embodiment 13: The system and/or method of any one of Embodiments 1 to 12, or any combination thereof, wherein the disorder is selected from the group consisting of: Multiple Sclerosis, Amyotrophic Lateral Sclerosis, Alzheimer's Disease, Tics, Parkinson's Disease, Huntington's Disease, Muscular Dystrophy, Cerebral Palsy, Stroke, Myasthenia Gravis, Peripheral Neuropathy, Ataxia, Friedreich's Ataxia, Dystonia, Restless Leg Syndrome, Polio (Poliomyelitis), Guillain-Barré Syndrome, Post-Polio Syndrome, Rheumatoid Arthritis, Osteoarthritis, Lupus, Tardive Dyskinesia, Chorea, Hemiballismus, Wilson's Disease, Brachial Plexus Injury, Tetanus, Motor Neuron Disease, Bell's Palsy, Essential Tremor, Orthostatic Tremor, Rett Syndrome, Spinocerebellar Ataxia, Spinal Muscular Atrophy, Primary Lateral Sclerosis (PLS), Charcot-Marie-Tooth Disease, Complex Regional Pain Syndrome (CRPS), Fibromyalgia, Progressive Supranuclear Palsy, Myoclonus, Phantom Limb Pain, Syringomyelia, Trigeminal Neuralgia, Osteoporosis, Ankylosing Spondylitis, Gout, Paget's Disease of Bone, Lyme Disease, Botulism, Tourette's Syndrome, Prion Diseases, Creutzfeldt-Jakob Disease, Stiff Person Syndrome (SPS), Dermatomyositis, Scleroderma, Batten Disease, Narcolepsy, Chronic Fatigue Syndrome (CFS), Machado-Joseph Disease, Benign Essential Blepharospasm, Foot Drop, Carpal Tunnel Syndrome, Peripheral Artery Disease, Reflex Sympathetic Dystrophy Syndrome, Pantothenate Kinase-Associated Neurodegeneration (PKAN), Mitochondrial Myopathies, Paraneoplastic Syndromes of the Nervous System, Chronic Inflammatory Demyelinating Polyneuropathy (CIDP), Progressive Multifocal Leukoencephalopathy, Transverse Myelitis, Myotonic Dystrophy, Cervical Spondylosis, Behçet's Disease, Pseudotumor Cerebri, Krabbe Disease, Neurofibromatosis, Acoustic Neuroma, Vestibular Neuritis and Labyrinthitis, Vertigo, Meniere's Disease, Chronic Paroxysmal Hemicrania, Antiphospholipid Syndrome (APS), Neuralgia, Paralysis, Postural Orthostatic Tachycardia Syndrome (POTS), Shy-Drager Syndrome, Vasculitis, Hemifacial Spasm, Isaacs' Syndrome, Marfan Syndrome, Osteogenesis Imperfecta, Ehlers-Danlos Syndromes, Alkaptonuria, Spasticity, Athetosis, Hyperkinesias, Hypokinesias, Meralgia Paresthetica, Restless Arms Syndrome, Piriformis Syndrome Spinal Cord Injury, Traumatic Brain Injury, Drug Craving, Food Craving, Alcohol Craving, Nicotine Craving, Craving, Addiction, Drug Abuse, Brain Injury, Diabetes, Cardiovascular Condition, Pulmonary Condition, Balance Ailment, Impingement Syndromes, Joint Replacement, Bone Fusion, bone fracture, joint injury, Trauma, Peripheral Nerve Injury, Post Surgery Injury, Declined Motor Performance, Stuttering, Tinnitus, Spasticity, Parkinsonianism, Obsessions, Depression, Schizophrenia, Bipolar Disorder, Acute Mania, Catatonia, Post-Traumatic Stress Disorder, Stroke, Cognitive Decline, Motor Dysfunction, Motor Performance Decline, Autism, Chronic Pain Syndrome, Epilepsy, Stroke, Auditory Hallucinations, Movement Disorders, Neurodegenerative Disorders, Pain Disorders, Metabolic Disorders, Addictive Disorders, Psychiatric Disorders, Traumatic Nerve Injury, and/or Sensory Disorders.
Embodiment 14: The system and/or method of any one of Embodiments 1 to 13, or any combination thereof, wherein the management plan is a therapy management plan communicated to a physical therapist.
Embodiment 15: The system and/or method of any one of Embodiments 1 to 14, or any combination thereof, further comprising performing physical therapy on the patient based on the therapy management plan; obtaining additional kinematic and/or kinetic information of the patient at a subsequent point in time and updating the therapy management plan based on the additional kinematic and/or kineticinformation.
Embodiment 16: The system and/or method of any one of Embodiments 1 to 15, or any combination thereof, wherein the kinematic and/or kinetic information is obtained while the patient is performing at least one of upper limb motor tasks, lower limb motor tasks, walking, standing still, or combinations thereof.
Embodiment 17: The system and/or method of any one of Embodiments 1 to 16, or any combination thereof, wherein the kinematic and/or kinetic information assesses at least one of bradykinesia, tremor, postural instability, or gait.
Embodiment 18: The system and/or method of any one of Embodiments 1 to 17, or any combination thereof, wherein assessing comprises diagnosing the subject with a movement disorder.
Embodiment 19: The system and/or method of any one of Embodiments 1 to 18, or any combination thereof, wherein assessing comprises determining severity of an existing disorder of the subject.
Embodiment 20: The system and/or method of any one of Embodiments 1 to 19, or any combination thereof, wherein the method is performed at least one additional time at a later point in time.
Embodiment 21: The system and/or method of any one of Embodiments 1 to 20, or any combination thereof wherein prior to the obtaining step, the method further comprises providing stimulation of tissue (e.g., neural, muscular, epithelial, connective, cardiac, endocrine, mucosal, pulmonary, lymphatic, skeletal) of the subject.
Embodiment 22: The system and/or method of any one of Embodiments 1 to 21, or any combination thereof, wherein the method is repeated after the subject has received stimulation of their tissue.
Embodiment 23: The system and/or method of any one of Embodiments 1 to 22, or any combination thereof, wherein the stimulation is non-invasive transcranial stimulation.
Embodiment 24: The system and/or method of any one of Embodiments 1 to 23, or any combination thereof, wherein the stimulation comprises a combination of electrical and mechanical stimulation.
Embodiment 25: The system and/or method of any one of Embodiments 1 to 24, or any combination thereof, further comprising conducting a clinical examination, wherein results of the examination are used in the determining step.
Embodiment 26: The system and/or method of any one of Embodiments 1 to 25, or any combination thereof, further comprising integrating Big Data.
Embodiment 27: The system and/or method of any one of Embodiments 1 to 26, or any combination thereof, further comprising integrating AI and statistical methods to, for example, drive the analysis, response generation, and/or patient communication.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Accordingly, these and other objects, along with advantages and features of the present disclosure will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention and are not intended as a definition of the limits of the invention. For purposes of clarity, not every component may be labeled in every drawing. In the following description, various embodiments of the present disclosure are described with reference to the following drawings, in which:

FIG. 1 is an illustration showing an embodiment of a motion analysis system of the present disclosure with an included analysis and prediction suite;

FIG. 2 is a flow chart illustrating steps performed by the processor for assessing a movement disorder;

FIG. 3 is an illustration of an exemplary accelerometer useful in the present disclosure;

FIG. 4 is an illustration of an exemplary gyroscope useful in the present disclosure;

FIG. 5A is an illustration showing exemplary placement of various components of the external body motion sensor for the hand;

FIG. 5B is an illustration showing an alternative exemplary placement of various components of the external body motion sensor for the hand;

FIG. 6A is a graph showing position data recorded from a camera device indicating the position of the wrist in space, provided in X, Y, Z coordinates in the space of the subject, in the units of meters, during a test is provided, where the blue line corresponds to the right wrist and the red line to the left wrist;

FIG. 6B illustrates information from accelerometers, provided in the X, Y, Z coordinates in the space relative to the accelerometer (i.e., relative to the measurement device) in relative units of the accelerometer;

FIG. 6C illustrates information from a gyroscope in relative units of the gyroscope;

FIG. 6D illustrates information of the velocity of movement, provided in X, Y, Z coordinates in the space of the subject, with the units of m/s, calculated based on the camera data of the right wrist;

FIG. 6E illustrates information of the velocity (red line) based on the camera information in line with the data simultaneously recorded with the accelerometer (blue line);

FIG. 6F is a table showing results for a continuous flexion extension task obtained using systems and methods of the disclosure;

FIG. 7 is a table showing results for a discrete flexion extension task obtained using systems of the disclosure;

FIG. 8A is a graph showing stability data of the position of the hand;

FIG. 8B illustrates peaks of the rotational component of the gyroscope along its X axis that are identified and displayed to the user (blue line in units of the gyroscopic device), with the red lines showing the triggering device, and the green line demonstrating the peak locations of the movements;

FIG. 8C (top half) shows data gathered with the hand held at the shoulder, and FIG. 8C (bottom half) is the same data for the hand held at the waist;

FIG. 9A is a graph showing an example of position data recorded by a camera provided in X, Y, Z coordinates in the space of the subject, where the blue line corresponds to the right wrist and the red line to the left wrist;

FIG. 9B is a graph showing velocity determined from the camera data (red), accelerometer data (blue line), and the trigger data to mark the beginning of the first and last movement (black lines). The y axis is given in m/s for the velocity data;

FIG. 9C is a graph showing data from the power in the movements of the right hand as a function of frequency as determined from the accelerometer data;

FIG. 9D is a table showing results obtained using systems of the disclosure for the task of a subject touching their nose;

FIG. 9E is a table showing results obtained using systems of the disclosure for the task of a subject touching their nose for the purpose of measuring tremor;

FIG. 10A, is a graph showing the weight calculated for the front and back of the left and right foot (in kg), the red line depicts a trigger mark where a clinician has determined a patient has stepped on the board and begun balancing and the second line depicts when the clinician tells the patient the test is over and they can prepare to get off the force plate, the x-axis is in until of time;

FIG. 10B is a graph showing a typical examples of data depicting patients center of gravity movements (blue), here depicted in units of length, and area ellipses depicting total movement (red) the top part shows a patient who has been perturbed (eyes open) and swaying and the bottom part shows a patient standing without perturbation (eyes closed). The time information could be communicated on a third axis or via color coding, here for clarity it is removed in the current depiction;

FIG. 10C is a graph showing jerk data, in units of position per time cubed, where the top part shows a patient who has been perturbed and swaying (eyes open) and the bottom part shows a patient standing without perturbation (eyes closed);

FIG. 10D is a set of two tables showing results. FIG. 10D (top table) shows eyes open and eyes closed data obtained while a subject is standing unperturbed. FIG. 10D (bottom table) shows eyes open data obtained while a subject is being pulled;

FIG. 11A is a graph showing peaks of the rotational component of the gyroscope along its Z axis, identified and displayed to the user (blue line in units of the gyroscopic device), where the red lines show the triggering device and the green line depicts the time instants corresponding to peaks of Z rotational component. The Y-axis is given in the relative units of the gyroscope around its Z-axis, and the X-axis in units of time and the triggering device here is activated on every step;

FIG. 11B shows the compiled results of the from the data shown in FIG. 11A, demonstrating the total walk time, and longest time per right step (Peak Distance);

FIG. 11C an example of Jerk (the Y-axis is in the units of m/time\hat{ }3, X-axis in terms of time), where the blue line corresponds to the period while a person is walking and the open space when the walk and task recording has stopped;

FIG. 11D shows the compiled results of the from data shown in FIG. 11C;

FIG. 12A is a table showing results obtained using systems of the disclosure for a subject performing a continuous flexion extension task;

FIG. 12B is a table showing results obtained using systems of the disclosure for a subject performing a discrete flexion extension task;

FIG. 12C is a table showing results obtained using systems of the disclosure for a subject performing a hand opening and closing task while the arm is positioned at the shoulder;

FIG. 12D is a table showing results obtained using systems of the disclosure for a subject performing a hand opening and closing task while the arm is positioned at the waist;

FIG. 12E is a table showing results obtained using systems of the disclosure for a subject performing the task of touching their nose;

FIG. 12F is a table showing results obtained using systems of the disclosure for a subject performing while the subject is asked to stand still;

FIG. 12G is a table showing results obtained using systems of the disclosure for a subject performing while the subject is walking;

FIG. 13A is a table showing a set of defined criteria for making a differential diagnosis of progressive supranuclear palsy (PSP) compared to other potential movement disorders;

FIG. 13B is a table showing symptoms demonstrated in 103 cases progressive supranuclear palsy, in early and later stages, which can be used to make a model for aiding in diagnosing the disease;

FIGS. 13C-G are a set of neuro-exam based flow charts based on statistical analysis for diagnosing a movement disorder;

FIG. 14 is a flowchart illustrating steps performed by the system for assessing a movement disorder, predicting a patient clinical scale that characterizes the movement disorder, and optimizing the processes and system used to assess, diagnose, classify, predict, or direct treatment of patients;

FIG. 15 is a flowchart illustrating steps performed by a set of motion analysis suites and a central computational system for optimizing the computational processes used to assess, diagnose, classify, predict, or direct treatment of patients;

FIG. 16 is a flowchart illustrating steps performed by a set of motion analysis suites, a central computational system, and a database for optimizing the computational processes used to assess, diagnose, classify, predict, or direct treatment of patients;

FIG. 17 is a flowchart illustrating steps performed by a set of motion analysis suites, secondary computational systems, and a central computational system, for optimizing the computational processes used to assess, diagnose, classify, predict, or direct treatment of patients;

FIG. 18 is an illustration showing an embodiment of a motion analysis suite of the disclosure that we used for assessing Parkinson's Disease patients;

FIG. 19 shows exemplary speed profiles of a flexion/extension task recorded from a patient with little motor impairment (A) and from a more impaired patient (B). The profiles displayed in A show clear speed minima, i.e., clear single movement starts and stops, differently from the speed profiles displayed in B for which gyroscope recordings are needed to determine the start and stop of each movement. The bottom half of the figure shows an expanded view of the segmented movements (indicated by the black vertical lines) from the speed profile where the gyroscope data (red) is overlaid on the camera data (blue);

FIG. 20 shows exemplary data recorded from two PD patients with different ability to control body posture as measured by the force plate. CoP trajectory of a patient with UPDRS III=18 (path length=142.7 cm) and of a patient with UPDRS III=8 (path length=23.57 cm) are shown in the left and right panel, respectively;

FIG. 21 shows exemplary Principal Component Analysis (PCA) results. The percentage of total variability as a function of the number of PC retained for the UPDRS III, motion analysis suite, and combined data sets is displayed. Number 1 and 2 refer to the two different recording sessions;

FIG. 22 shows exemplary prediction data;

FIG. 23A shows a step in a predictive process using the motion analysis system computational elements. A LASSO based model of UPDRS3 prediction is assessed as a function of its degrees of freedom (of motion analysis system metrics). Here we demonstrate the mean UPDRS predictive error as of function of degrees of freedom. This data was derived in the testing of 50 Parkinson's Disease patients;

FIG. 23B shows a step in a predictive process using the motion analysis system computational elements. A LASSO based model of UPDRS3 prediction is assessed as a function of its degrees of freedom (of motion analysis system metrics). Here we demonstrate the variance explained by the model as of function of degrees of freedom. This data was derived in the testing of 50 Parkinson's Disease patients;

FIG. 23C shows a step in a predictive process using the motion analysis system computational elements. A 1-out Cross Validation of the UPDRS3 predictions is demonstrated, with a mean error of less 0.5. This data was derived in the testing of 50 Parkinson's Disease patients;

FIG. 24A shows an implementation of a motion analysis system for assessing diabetic neuropathic pain patients undergoing two different treatments as can be used for comparing or optimizing the treatments. Here we look a difference in patients' Functional Reach testing;

FIG. 24B shows an implementation of a motion analysis system for assessing diabetic neuropathic pain patients undergoing two different treatments as can be used for comparing or optimizing the treatments. Here we look a difference in patients' Single Leg Balance testing;

FIG. 25 shows a Software Process Example view of one embodiment of software computational module for an RCT Design Analysis tool to optimize value in a trial design;

FIG. 26 shows a chart outlining how one could use Sinusoidal Steady State Solutions of the electromagnetic fields during brain stimulation (such as transcranial magnetic stimulation (TMS) and deep brain stimulation (DBS)) that can determined from MRI derived Finite Element Models based on frequency specific tissue electromagnetic properties of head and brain tissue. The sinusoidal steady state solutions can be transformed into the time domain to rebuild the transient solution for the stimulation dose in the targeted brain tissues. These solutions can then be coupled with single cell conductance-based models of neurons to explore the electrophysiological response to stimulation. High resolution patient specific models can be developed, implementing more complicated biophysical modeling (e.g., coupled electromechanical field models or any typical energy) and be used as part of large heterogenous data sets (e.g., clinical, imaging, and kinematics) to optimize/tune therapy (such as with a system of motion analysis suite(s) and neural stimulation dose controller);

FIG. 27 shows a schematic of an exemplary motion analysis suite for delivering personalized treatments based on the motion analysis suite(s) and a big data infrastructure, whereby multimodal data sets (e.g., imaging, biophysical field-tissue interaction models, clinical, biospecimen data) can be coupled to deliver personalized brain stimulation-based treatments in a diverse and expansive patient cohort. Each integrated step can be computationally intensive (e.g., see FIG. 26 for simplified dosing example for exemplary electromagnetic brain stimulation devices). This same schematic can be used for guiding and optimizing other therapies (e.g., physical therapy, balance training, rehabilitation training). Finally, while these images and descriptions often center on the motion analysis suite(s), the same methods could be applied in a system with a motion analysis core (e.g., a brain stimulation based therapeutic system with the same elements shown in the figure, yet without the motion analysis component). Furthermore, the methods could be applied with multiple systems, integrated together as outlined herein (see, e.g., FIG. 15-17); and

FIG. 28 is a flowchart showing the steps for generating an optimal physical therapy/exercise program personalized for that patient. Specific patient motor abilities are assessed with the motion suite. Motor abilities can be described in a modular way (for example in terms of movement speed, balance, or gait); a specific ability is associated to a specific training module (e.g., training exercises specific for movement speed, balance, or gait). If a specific motor ability is impaired, the associated training video is selected. An exercise program is generated by combining the different videos, where the parameters of each exercise (e.g., number of repetitions) are calculated by an algorithm that considers various variables (total duration of session, priority (e.g., based on predefined knowledge or more severely affected ability).

### DETAILED DESCRIPTION

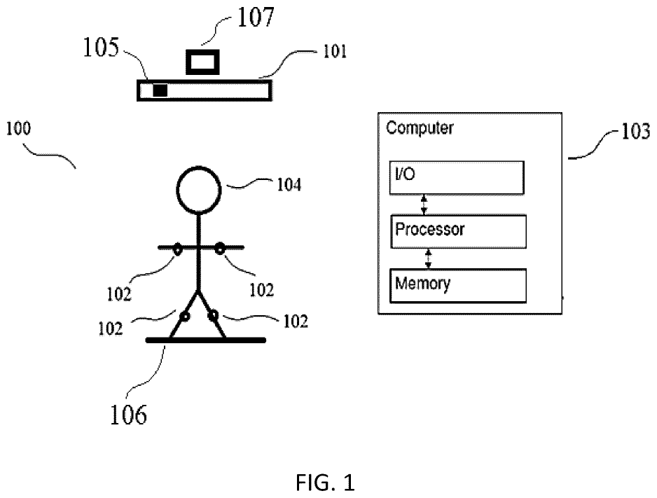

The disclosure generally relates to motion analysis suite and methods of use thereof. FIG. 1 shows an exemplary motion analysis system 100. The system 100 includes an image capture device 101, at least one external body motion sensor 102, and a central processing unit (CPU) 103 with storage coupled thereto for storing instructions that when executed by the CPU cause the CPU to receive a first set of motion data from the image capture device related to at least one joint of a subject 104 while the subject 104 is performing a task and receive a second set of motion data from the external body motion sensor 102 related to the at least one joint of the subject 104 while the subject 104 is performing the task. The CPU 103 also calculates kinematic and/or kinetic information about the at least one joint of a subject 104 from a combination of the first and second sets of motion data, and outputs the kinematic and/or kinetic information for purposes of assessing a movement disorder. In certain alternative embodiments, more than one image capture device can be used.

Systems of the disclosure include software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program(s) include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. Examples of communication networks include cell network (e.g., 3G, 4G, or 5G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, C#, Perl, Python, Matlab), and it can be deployed in any form, including as a stand-alone program or as a module, component, subroutine, or other unit suitable for use in a computing environment. Systems and methods of the disclosure can include instructions written in any suitable programming language known in the art, including, without limitation, C, C++, C#, Perl, Java, Python, ActiveX, Assembly, Matlab, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the disclosure involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information, and which can be accessed by a computing device.

As one skilled in the art would recognize as necessary or best-suited for performance of the methods of the disclosure, a computer system or machines of the disclosure include one or more processors (e.g., a central processing unit (CPU) a graphics processing unit (GPU) or both), a main memory and a static memory, which communicate with each other via a bus.

In the exemplary embodiment shown in FIG. 1, system 100 can include a computer 103 (e.g., laptop, desktop, watch, smart phone, or tablet). The computer 103 may be configured to communicate across a network to receive data from image capture device 101 and external body motion sensors 102. The connection can be wired or wireless. Computer 103 includes one or more processors and memory as well as an input/output mechanism(s). In certain embodiments, systems of the disclosure employ a client/server architecture, and certain processing steps of sets of data may be stored or performed on the server, which may include one or more of processors and memory, capable of obtaining data, instructions, etc., or providing results via an interface module or providing results as a file. Server may be engaged over a network through computer 103.

System 100 or machines according to the disclosure may further include, for any I/O, a video display unit (e.g., a liquid crystal display (LCD) or a cathode ray tube (CRT)). Computer systems or machines according to the disclosure can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory according to the disclosure can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

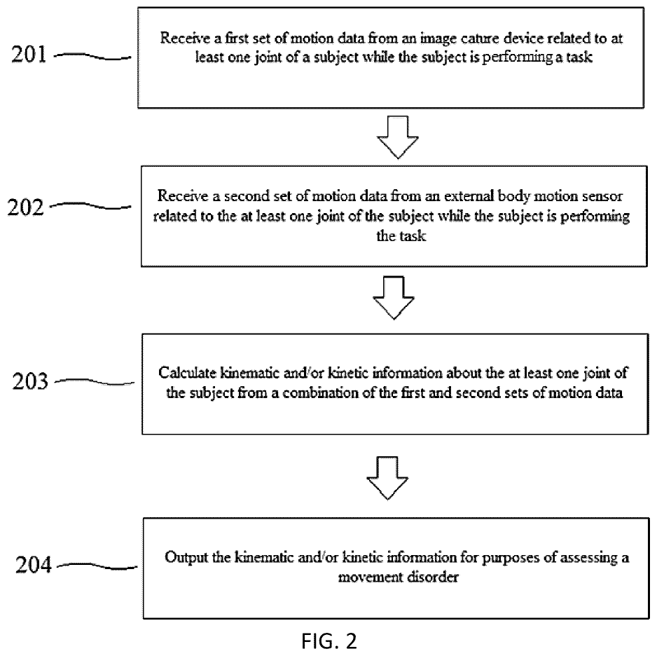

Exemplary step-by-step methods are described schematically in FIG. 2. It will be understood that of the methods described in FIG. 2, as well as any portion of the systems and methods disclosed herein, can be implemented by computer, including the devices described above. At step 201, a first set of motion data from an image capture device is received to the CPU. The first set of motion data is related to at least one joint of a subject while the subject is performing a task. At step 202, a second set of motion data from the external body motion sensor is received to the CPU. The second set of motion data is related to the at least one joint of the subject while the subject is performing the task. In certain embodiments step 201 and step 202 can occur simultaneously in parallel and/or staggered in any order. At step 203, the CPU calculates kinematic and/or kinetic information about the at least one joint of a subject from a combination of the first and second sets of motion data. That calculation can be based on comparing the received data from the subject to a reference set that includes motion data from age and physiologically matched healthy individuals. The reference set of data may be stored locally within the computer, such as within the computer memory. Alternatively, the reference set may be stored in a location that is remote from the computer, such as a server. In that instance, the computer communicates across a network to access the reference set of data. The relative timing of step 201 and step 202 can be controlled by components in measurement devices and/or in the CPU system. At step 204, the CPU outputs the kinematic and/or kinetic information for purposes of assessing a movement disorder.

Additionally, in certain embodiments, patient data can be displayed on a device that the patient can observe (such as on a monitor, a phone, and/or a watch). This data can be used for self-evaluation and/or as part of a training and/or therapeutic regimen. In certain embodiments the data and/or analysis results could be communicated through wired or wireless methods to clinicians who can evaluate the data, such as for example remotely through telemedicine procedures. In certain embodiments the data to be transmitted could be compressed prior to transmitting from and/or to a sensor (e.g., camera, accelerometer) from and/or to a receiver in the CPU based system when information is communicated (either through wired or wireless communications). Such data can also be encrypted and/or protected prior, during, or after to transmitting and/or storing (internally in the sensor and/or at the CPU system). Such encryption methods and protection methods are exemplified herein, e.g., see below, or as those exemplified in FDA Cybersecurity Guidances, Cybersecurity Reports, and/or White Papers found at https://www.fda.gov/medical-devices/digital-health-center-excellence/cybersecurity. Any wired or wireless communication standard can be used, such low energy Bluetooth, Zigbee (an IEEE 802.15.4 based specification of protocols), and passive wi-fi can be implemented.

### Image Capture Device

Numerous different types of image capture devices can be used in systems of the disclosure. An exemplary image capture device and its software is described, for example, in U.S. pat. publ. nos. 2010/0199228; 2010/0306716; 2010/0306715; 2010/0306714; 2010/0306713; 2010/0306712; 20100306671; 2010/0306261; 2010/0303290; 2010/0302253; 2010/0302257; 2010/0306655; and 2010/0306685, the content of each of which is incorporated by reference herein in its entirety. An exemplary image capture device is the Microsoft Kinect (commercially available from Microsoft).

The image capture device 101 will typically include software for processing the received data from the subject 104 before transmitting the data to the CPU 103. The image capture device and its software enable advanced gesture recognition, facial recognition and optionally voice recognition. The image capture device is able to capture a subject for motion analysis with a feature extraction of one or more joints, e.g., 1 joint, 2 joints, 3 joints, 4 joints, 5 joints, 6 joints, 7 joints, 8 joints, 9 joints, 10 joints, 15 joints, or 20 joints.

In certain embodiments, the hardware of the image capture device includes a range camera that in certain embodiments can interpret specific gestures and/or movements by using an infrared projector and camera. The image capture device may be a horizontal bar connected to a small base with a motorized pivot. The device may include a red, green, and blue (RGB) camera, and depth sensor, which provides full-body 3D motion capture and facial recognition. The image capture device can also optionally include a microphone 105 for capture of sound data (such as for example for voice recordings or for recording sounds from movements). Alternatively, the microphone or similar voice capture device may be separate from the image capture device. The depth sensor may include an infrared laser projector combined with a monochrome CMOS sensor, which captures video data in 3D under any ambient light conditions. The sensing range of the depth sensor is adjustable, and the image capture software is capable of automatically calibrating the sensor-based on a subject's physical environment, accommodating for the presence of obstacles. Alternatively, the camera may also capture thermal and/or infrared data. In alternative embodiments sound data can be used for localizing positions, such as would be done in a SONAR method with sonic and/or ultrasonic data. In certain embodiments, the system could employ Radio Detection and Ranging (RADAR) technology as part of the localizing step.

In certain embodiments, the image capture device is worn on the subject, such as with a GO PRO camera (commercially available from GO Pro). In certain embodiments, the subject wears a light or a light reflecting marker to increase image clarity and/or contrast. In certain embodiments, the system makes use of a camera capable of being attached to the internet.

The software of the image capture device tracks the movement of objects and individuals in three dimensions. The image capture device and its software use structured light and machine learning. To infer body position, a two-stage process is employed. First a depth map (using structured light) is computed, and then body position (using machine learning) is inferred.

The depth map is constructed by analyzing a speckle pattern of infrared laser light. Exemplary techniques for constructing such a depth map are described, for example, in U.S. pat. publ. nos. 2011/0164032; 2011/0096182; 2010/0290698; 2010/0225746; 2010/0201811; 2010/0118123; 2010/0020078; 2010/0007717; and 2009/0185274, the content of each of which is incorporated by reference herein in its entirety. Briefly, the structured light general principle involves projecting a known pattern onto a scene and inferring depth from the deformation of that pattern. Image capture devices described herein uses infrared laser light, with a speckle pattern. The depth map is constructed by analyzing a speckle pattern of infrared laser light. Data from the RGB camera is not required for this process.

The structured light analysis is combined with a depth from focus technique and a depth from stereo technique. Depth from focus uses the principle that objects that are more blurry are further away. The image capture device uses an astigmatic lens with different focal length in x- and y directions. A projected circle then becomes an ellipse whose orientation depends on depth. This concept is further described, for example in Freedman et al. (U.S. pat. publ. no. 2010/0290698), the content of which is incorporated by reference herein in its entirety.

Depth from stereo uses parallax. That is, if you look at the scene from another angle, objects that are close get shifted to the side more than objects that are far away. Image capture devices used in systems of the disclosure analyze the shift of the speckle pattern by projecting from one location and observing from another.

Next, body parts are inferred using a randomized decision forest, learned from over many training examples, e.g., 1 million training examples. Such an approach is described for example in Shotten et al. (CVPR, 2011), the content of which is incorporated by reference herein in its entirety. That process starts with numerous depth images (e.g., 100,000 depth images) with known skeletons (from the motion capture system). For each real image, dozens more are rendered using computer graphics techniques. For example, computer graphics are used to render all sequences for 15 different body types, and while varying several other parameters, which obtains over a million training examples.

In the next part of this process, depth images are transformed to body part images. That is accomplished by having the software learn a randomized decision forest, and mapping depth images to body parts. Learning of the decision forest is described in Shotten et al. (CVPR, 2011). In the next part of the process, the body part image is transformed into a skeleton, which can be accomplished using mean average algorithms.

Image recording can be accomplished via methods such as those described above, the examples of which should not be considered limiting but exemplary, and image acquisition can be accomplished by other mechanisms such as those described in (The Image Processing Handbook, JC Russ and FB Neal, 2017, CRC Press) or (Image Acquisition, M W Burke, 1996, Springer, Dordrecht) and use for example optical (e.g., laser), electromagnetic (e.g., visual spectrum, infrared spectrum, etc.), thermal, and/or acoustic signals for image acquisition and processing. Similarly, image processing and computer vision for creating and tracking the skeleton can be accomplished via methods such as those described above, the examples of which should not be considered limiting but exemplary, and by any other image processing algorithms that can improve the visual qualities of the image (including but not limited to image denoising, camera calibration, improvement of signal to noise/ratio), accomplish boundary extraction and image segmentation; track image features (e.g., in a video); accomplish scene understanding, e.g., using methods for object recognition, 3D reconstruction, texture analysis, and learning algorithms including neural networks such as Radial Basis Function (RBF) networks, self-organizing maps (SOM), Hopfield networks, deep neural networks; generative adversarial networks or any other method for supervised or unsupervised learning (Handbook of Image Processing and Computer Vision: Volume 1: from Energy to Image, A. Distante, C. Distante, 2020, Springer; Handbook of Image Processing and Computer Vision: Volume 2: from Image to Pattern, A. Distante, C. Distante, 2020, Springer; Handbook of Image Processing and Computer Vision: Volume 3: from Pattern to Object, A. Distante, C. Distante, 2020, Springer; Advanced Methods and Deep Learning in Computer Vision (Computer Vision and Pattern Recognition), E. R. Davies, M. Turk, 2022, Academic Press).

### External Body Motion Sensor

Many types of external body motion sensors are known by those skilled in the art for measuring external body motion. Those sensors include but are not limited to accelerometers, gyroscopes, magnetometers, goniometer, resistive bend sensors, combinations thereof, and the like. In certain embodiments, an accelerometer is used as the external body motion sensor. In other embodiments, a combination using an accelerometer and gyroscope is used. Exemplary external body motion sensors are described for example in U.S. Pat. Nos. 8,845,557; 8,702,629; 8,679,038; and 8,187,209, the content of each of which is incorporated by reference herein in its entirety. The system of the disclosure can use one or more external body motion sensors, and the number of sensors used will depend on the number of joints to be analyzed, typically 1 sensor per joint, although in certain embodiments, 1 sensor can analyze more than one joint. For example, one or more joints can be analyzed using one or more sensors, e.g., 1 joint and 1 sensor, 2 joints and 2 sensors, 3 joints and 3 sensors, 4 joints and 4 sensors, 5 joints and 5 sensors, 6 joints and 6 sensors, 7 joints and 7 sensors, 8 joints and 8 sensors, 9 joints and 9 sensors, 10 joints and 10 sensors, 15 joints and 15 sensors, or 20 joints and 20 sensors.

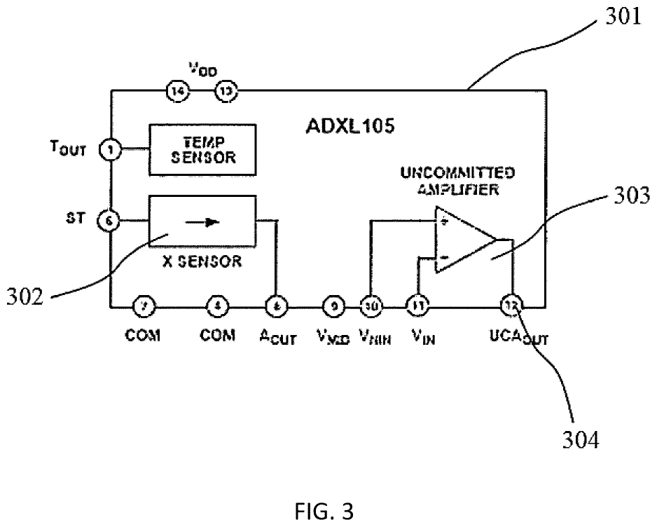

In certain embodiments, external body motion sensor 102 is an accelerometer. FIG. 3 is an electrical schematic diagram for one embodiment of a single axis accelerometer of the present disclosure. The accelerometer 301 is fabricated using a surface micro-machining process. The fabrication technique uses standard integrated circuit manufacturing methods enabling all signal processing circuitry to be combined on the same chip with the sensor 302. The surface micro-machined sensor element 302 is made by depositing polysilicon on a sacrificial oxide layer that is then etched away leaving a suspended sensor element. A differential capacitor sensor is composed of fixed plates and moving plates attached to the beam that moves in response to acceleration. Movement of the beam changes the differential capacitance, which is measured by the on chip circuitry. All the circuitry 303 needed to drive the sensor and convert the capacitance change to voltage is incorporated on the chip requiring no external components except for standard power supply decoupling. Both sensitivity and the zero-g value are ratiometric to the supply voltage, so that ratiometric devices following the accelerometer (such as an analog to digital converter (ADC), etc.) will track the accelerometer if the supply voltage changes. The output voltage (VOUT) 304 is a function of both the acceleration input and the power supply voltage (VS).

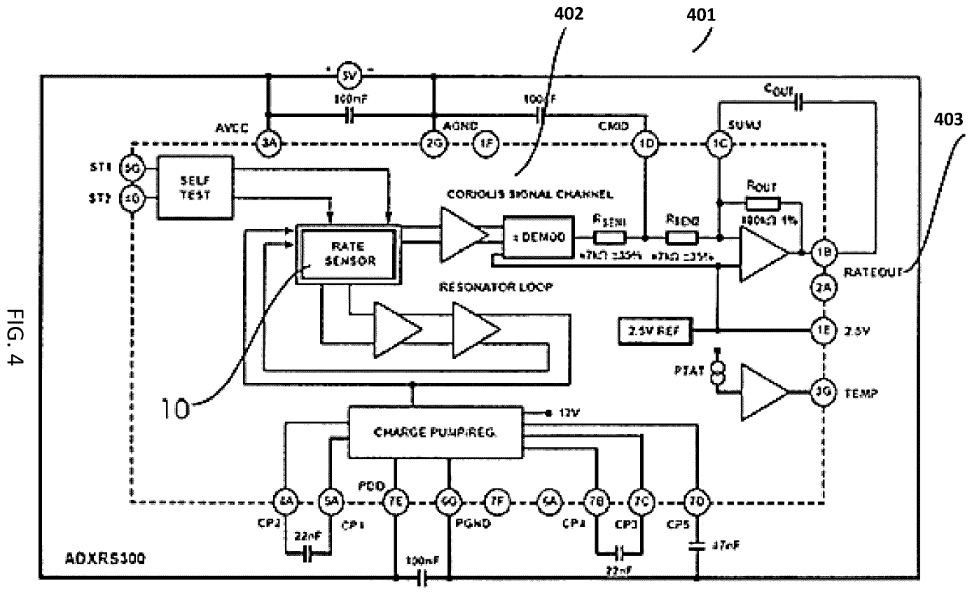

In certain embodiments, external body motion sensor 102 is a gyroscope. FIG. 4 is an electrical schematic diagram for one embodiment of a gyroscope 401 used as a sensor or in a sensor of the present disclosure. The sensor element functions on the principle of the Coriolis Effect and a capacitive-based sensing system. Rotation of the sensor causes a shift in response of an oscillating silicon structure resulting in a change in capacitance. An application specific integrated circuit (ASIC) 402, using a standard complementary metal oxide semiconductor (CMOS) manufacturing process, detects and transforms changes in capacitance into an analog output voltage 403, which is proportional to angular rate. The sensor element design utilizes differential capacitors and symmetry to significantly reduce errors from acceleration and off-axis rotations.

The accelerometer and/or gyroscope can be coupled to or integrated within into a kinetic sensor board, such as that described in U.S. Pat. No. 8,187,209, the content of which is incorporated by reference herein in its entirety. Therefore, certain embodiments are just an accelerometer and a kinetic sensor board, other embodiments are just a gyroscope and a kinetic sensor board, and still other embodiments are a combination of an accelerometer and a gyroscope and a kinetic sensor board. The kinetic sensor board may include a microprocessor (e.g., Texas Instruments mSP430-169) and a power interface section.

The kinetic sensor board and accelerometer and/or gyroscope can be further coupled to or integrated within a transceiver module, such as that described in U.S. Pat. No. 8,187,209. The transceiver module can include a blue tooth radio (EB100 A7 Engineering) to provide wireless communications with the CPU 103, and data acquisition circuitry, on board memory, a microprocessor (Analog Devices ADVC7020), and a battery power supply (lithium powered) that supplies power to both the transceiver module and one or more external sensor modules. The transceiver module also includes a USB port to provide battery recharging and serial communications with the CPU 103. The transceiver module also includes a push button input.

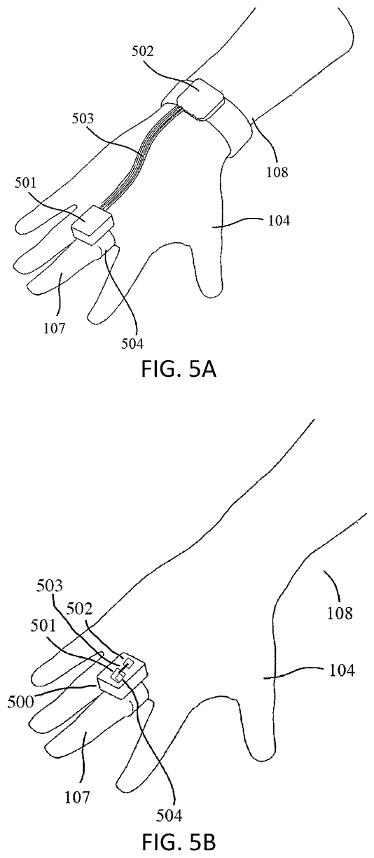

FIG. 5A illustrates one possible embodiment of the subject 104 worn components of the system combining the sensor board 501 and the transceiver module 502. The sensor board 501 consists of at least one accelerometers 504. The sensor board 501 is worn on the subject's 104 finger 106 and the transceiver module 502 is worn on the subject's 104 wrist 108. The transceiver module 502 and one or more external sensor modules 501 are connected by a thin multi-wire leads 503. In an alternative embodiment, all of the components are made smaller and housed in a single housing chassis 500 that can be mounted on or worn by the subject at one location, say for example all are worn on the finger in a single housing chassis 500, FIG. 5B. In an alternative embodiment, the accelerometer (and/or other motion analysis sensors (e.g., gyroscope)) could be housed in a mobile computing device worn on the subject, such as for example a mobile phone.

In operation, the input to the external sensor module consists of the kinetic forces applied by the user and measured by the accelerometers and/or gyroscopes. The output from the board is linear acceleration and angular velocity data in the form of output voltages. These output voltages are input to the transceiver module. These voltages undergo signal conditioning and filtering before sampling by an analog to digital converter. This digital data is then stored in on board memory and/or transmitted as a packet in RF transmission by a blue tooth transceiver. A microprocessor in the transceiver module controls the entire process. Kinetic data packets may be sent by RF transmission to nearby CPU 103 which receives the data using an embedded receiver, such as blue tooth or other wireless technology. A wired connection can also be used to transmit the data. Alternatively, Kinetic data may also be stored on the on board memory and downloaded to CPU 103 at a later time. The CPU 103 then processes, analyzes, and stores the data.

In certain embodiments, the kinetic sensor board includes at least three accelerometers and measures accelerations and angular velocities about each of three orthogonal axes. The signals from the accelerometers and/or gyroscopes of the kinetic sensor board are preferably input into a processor for signal conditioning and filtering. Preferably, three Analog Devices gyroscopes (e.g., ADXRS300) are utilized on the kinetic sensor board with an input range up to 1200 degrees/second. The ball grid array type of component may be selected to minimize size. Additionally, a MEMS technology dual axis accelerometer, from Analog Devices (ADXL210), may be employed to record accelerations along the x and y-axes. Other combinations of accelerometers and gyroscopes known to those skilled in the art could also be used. A lightweight plastic housing may then be used to house the sensor for measuring the subject's external body motion. The external body motion sensor(s) can be worn on any of the subject's joints or in close proximity of any of the subject's joints, such as on the subject's finger, hand, wrist, forearm, upper arm, head, chest, back, legs, feet and/or toes.

In certain embodiments, the transceiver module contains one or more electronic components such as the microprocessor for detecting both the signals from the gyroscopes and accelerometers. In certain embodiments, the one or more electronic components also filter (and possibly amplify) the kinetic motion signals, and more preferably convert these signals, which are in an analog form into a digital signal for transmission to the remote receiving unit. The one or more electronic components are attached to the subject as part of device or system. Further, the one or more electronic components can receive a signal from the remote receiving unit or other remote transmitters. The one or more electronic components may include circuitry for but are not limited to for example electrode amplifiers, signal filters, analog to digital converter, blue tooth radio, a DC power source, and combinations thereof. The one or more electronic components may comprise one processing chip, multiple chips, single function components or combinations thereof, which can perform all of the necessary functions of detecting a kinetic or physiological signal from the accelerometer and/or gyroscope, storing that data to memory, uploading data to a computer through a serial link, transmitting a signal corresponding to a kinetic or physiological signal to a receiving unit and optionally receiving a signal from a remote transmitter. These one or more electronic components can be assembled on a printed circuit board or by any other means known to those skilled in the art. Preferably, the one or more electronic components can be assembled on a printed circuit board or by other means so its imprint covers an area less than 4 in², more preferably less than 2 in², even more preferably less than 1 in², still even more preferably less than 0.5 in², and most preferably less than 0.25 in².

In certain embodiments, the circuitry of the one or more electronic components is appropriately modified so as to function with any suitable miniature DC power source. For example, the DC power source is a battery, such as lithium powered batteries. Lithium ion batteries offer high specific energy (the number of given hours for a specific weight), which is preferable. Additionally, these commercially available batteries are readily available and inexpensive. Other types of batteries include but are not limited to primary and secondary batteries. Primary batteries are not rechargeable since the chemical reaction that produces the electricity is not reversible. Primary batteries include lithium primary batteries (e.g., lithium/thionyl chloride, lithium/manganese dioxide, lithium/carbon monofluoride, lithium/copper oxide, lithium/iodine, lithium/silver vanadium oxide and others), alkaline primary batteries, zinc-carbon, zinc chloride, magnesium/manganese dioxide, alkaline-manganese dioxide, mercuric oxide, silver oxide as well as zinc/air and others. Rechargeable (secondary) batteries include nickel-cadmium, nickel-zinc, nickel-metal hydride, rechargeable zinc/alkaline/manganese dioxide, lithium/polymer, lithium-ion, and others. The power system and/or batteries may be rechargeable through inductive means, wired means, and/or by any other means known to those skilled in the art. The power system could use other technologies such as ultra-capacitors.

In certain embodiments, the circuitry of the one or more electronic components comprises data acquisition circuitry. The data acquisition circuitry is designed with the goal of reducing size, lowering (or filtering) the noise, increasing the DC offset rejection, and reducing the system's offset voltages. The data acquisition circuitry may be constrained by the requirements for extremely high input impedance, very low noise and rejection of very large DC offset and common-mode voltages, while measuring a very small signal of interest. Additional constraints arise from the need for a “brick-wall” style input protection against ESD and EMI. The exact parameters of the design, such as input impedance, gain and pass-band, can be adjusted at the time of manufacture to suit a specific application via a table of component values to achieve a specific full-scale range and pass-band.

In certain embodiments, a low-noise, lower power instrumentation amplifier is used. The inputs for this circuitry is guarded with preferably, external ESD/EMI protection, and very high-impedance passive filters to reject DC common-mode and normal-mode voltages. Still preferably, the instrumentation amplifier gain can be adjusted from unity to approximately 100 to suit the requirements of a specific application. If additional gain is required, it preferably is provided in a second-order anti-bias filter, whose cutoff frequency can be adjusted to suit a specific application, with due regard to the sampling rate. Still preferably, the reference input of the instrumentation amplifier is tightly controlled by a DC cancellation integrator servo that uses closed-loop control to cancel all DC offsets in the components in the analog signal chain to within a few analog-to digital converter (ADC) counts of perfection, to ensure long term stability of the zero reference.

In certain embodiments, the signals are converted to a digital form. This can be achieved with an electronic component or processing chip through the use of an ADC. More preferably, the ADC restricts resolution to 16-bits due to the ambient noise environment in such chips (other data resolutions can be used such as 8 bit, 32 bit, 64 bit, or more). Despite this constraint, the ADC remains the preferable method of choice for size-constrained applications such as with the present disclosure unless a custom data acquisition chip is used because the integration reduces the total chip count and significantly reduces the number of interconnects required on the printed circuit board.

In certain embodiments, the circuitry of the sensor board comprises a digital section. For example, the heart of the digital section of the sensor board is the Texas Instruments MSP430-169 microcontroller. The Texas Instruments MSP430-169 microcontroller contains sufficient data and program memory, as well as peripherals which allow the entire digital section to be neatly bundled into a single carefully programmed processing chip. Still preferably, the onboard counter/timer sections are used to produce the data acquisition timer.

In certain embodiments, the circuitry of the transceiver module comprises a digital section. For example, the heart of the digital section of the sensor board is the Analog Devices ADVC7020 microcontroller. The Analog Devices ADVC7020 microcontroller contains sufficient data and program memory, as well as peripherals which allow the entire digital section to be neatly bundled into a single carefully programmed processing chip. Still preferably, the onboard counter/timer sections are used to produce the data acquisition timer.

In certain embodiments, the circuitry for the one or more electronic components is designed to provide for communication with external quality control test equipment prior to sale, and more preferably with automated final test equipment. In order to supply such capability without impacting the final size of the finished unit, one embodiment is to design a communications interface on a separate PCB using the SPI bus with an external UART and level-conversion circuitry to implement a standard serial interface for connection to a personal computer or some other form of test equipment. The physical connection to such a device requires significant PCB area, so preferably the physical connection is designed to keep the PCB at minimal imprint area. More preferably, the physical connection is designed with a break-off tab with fingers that mate with an edge connector. This allows all required final testing and calibration, including the programming of the processing chip memory, can be carried out through this connector, with test signals being applied to the analog inputs through the normal connections which remain accessible in the final unit. By using an edge-finger on the production unit, and an edge connector in the production testing and calibration adapter, the system can be tested and calibrated without leaving any unnecessary electronic components or too large an PCB imprint area on the final unit.

In certain embodiments, the circuitry for the one or more electronic components comprises nonvolatile, rewriteable memory. Alternatively, if the circuitry for the one or more electronic components does not comprise nonvolatile, rewriteable memory then an approach can be used to allow for reprogramming of the final parameters such as radio channelization and data acquisition and scaling. Without nonvolatile, rewriteable memory, the program memory can be programmed only once. Therefore, one embodiment of the present disclosure involves selective programming of a specific area of the program memory without programming the entire memory in one operation. Preferably, this is accomplished by setting aside a specific area of program memory large enough to store several copies of the required parameters. Procedurally, this is accomplished by initially programming the circuitry for the one or more electronic components with default parameters appropriate for the testing and calibration. When the final parameters have been determined, the next area is programmed with these parameters. If the final testing and calibration reveals problems, or some other need arises to change the values, additional variations of the parameters may be programmed. The firmware of various embodiments of the present disclosure scans for the first blank configuration block and then uses the value from the preceding block as the operational parameters. This arrangement allows for reprogramming of the parameters up to several dozen times, with no size penalty for external EEPROM or other nonvolatile RAM. The circuitry for the one or more electronic components has provisions for in-circuit programming and verification of the program memory, and this is supported by the breakoff test connector. The operational parameters can thus be changed up until the time at which the test connector is broken off just before shipping the final unit. Thus, the manufacturability and size of the circuitry for the one or more electronic components is optimized.

In certain embodiments, the circuitry of the one or more electronic components includes an RF transmitter, such as a Wi-Fi based system and/or a blue tooth radio system utilizing the EB100 component from A7 engineering. Another feature of the circuitry of the one or more electronic components preferably is an antenna. The antenna, preferably, is integrated in the rest of the circuitry. The antenna can be configured in a number of ways, for example as a single loop, dipole, dipole with termination impedance, logarithmic-periodic, dielectric, strip conduction or reflector antenna. The antenna is designed to include but not be limited to the best combination of usable range, production efficiency and end-system usability. Preferably, the antenna consists of one or more conductive wires or strips, which are arranged in a pattern to maximize surface area. The large surface area will allow for lower transmission outputs for the data transmission. The large surface area will also be helpful in receiving high frequency energy from an external power source for storage. Optionally, the radio transmissions of the present disclosure may use frequency-selective antennas for separating the transmission and receiving bands, if a RF transmitter and receiver are used on the electrode patch, and polarization-sensitive antennas in connection with directional transmission. Polarization-sensitive antennas consist of, for example, thin metal strips arranged in parallel on an insulating carrier material. Such a structure is insensitive to or permeable to electromagnetic waves with vertical polarization; waves with parallel polarization are reflected or absorbed depending on the design. It is possible to obtain in this way, for example good cross polarization decoupling in connection with linear polarization. It is further possible to integrate the antenna into the frame of a processing chip or into one or more of the other electronic components, whereby the antenna is preferably realized by means of thin film technology. The antenna can serve to just transfer data or for both transferring data to and for receiving control data received from a remote communication station which can include but is not limited to a wireless relay, a computer, or a processor system. Optionally, the antenna can also serve to receive high-frequency energy (for energy supply or supplement). In any scenario, only one antenna is required for transmitting data, receiving data, and optionally receiving energy. The couplers being used to measure the radiated or reflected radio wave transmission output. Any damage to the antenna (or also any faulty adaptation) thus can be registered because it is expressed by increased reflection values.

An additional feature of the present disclosure is an optional identification unit. By allocating identification codes—a patient code, the remote communication station is capable of receiving and transmitting data to several subjects, and for evaluating the data if the remote communication station is capable of doing so. This is realized in a way such that the identification unit has control logic, as well as a memory for storing the identification codes. The identification unit is preferably programmed by radio transmission of the control characters and of the respective identification code from the programming unit of the remote communication station to the patient worn unit. More preferably, the unit comprises switches as programming lockouts, particularly for preventing unintentional reprogramming.

In any RF link, errors are an unfortunate and unavoidable problem. Analog systems can often tolerate a certain level of error. Digital systems, however, while being inherently much more resistant to errors, also suffer a much greater impact when errors occur. Thus, the present disclosure when used as a digital system, preferably includes an error control sub architecture. Preferably, the RF link of the present disclosure is digital. RF links can be one-way or two-way. One-way links are used to just transmit data. Two-way links are used for both sending and receiving data.

If the RF link is one-way error control, then this is preferably accomplished at two distinct levels, above and beyond the effort to establish a reliable radio link to minimize errors from the beginning. At the first level, there is the redundancy in the transmitted data. This redundancy is performed by adding extra data that can be used at the remote communication station or at some station to detect and correct any errors that occurred during transit across the airwaves. This mechanism known as Forward Error Correction (FEC) because the errors are corrected actively as the signal continues forward through the chain, rather than by going back to the transmitter and asking for retransmission. FEC systems include but are not limited to Hamming Code, Reed-Solomon and Golay codes. Preferably, a Hamming Code scheme is used. While the Hamming Code scheme is sometimes maligned as being outdated and underpowered, the implementation in certain embodiments of the present disclosure provides considerable robustness and extremely low computation and power burden for the error correction mechanism. FEC alone is sufficient to ensure that the vast majority of the data is transferred correctly across the radio link. Certain parts of the packet must be received correctly for the receiver to even begin accepting the packet, and the error correction mechanism in the remote communication station reports various signal quality parameters including the number of bit errors which are being corrected, so suspicious data packets can be readily identified and removed from the data stream.

Preferably, at a second, optional level, an additional line of defense is provided by residual error detection through the use of a cyclic redundancy check (CRC). The algorithm for this error detection is similar to that used for many years in disk drives, tape drives, and even deep-space communications, and is implemented by highly optimized firmware within the electrode patch processing circuitry. During transmission, the CRC is first applied to a data packet, and then the FEC data is added covering the data packet and CRC as well. During reception, the FEC data is first used to apply corrections to the data and/or CRC as needed, and the CRC is checked against the message. If no errors occurred, or the FEC mechanism was able to properly correct such errors as did occur, the CRC will check correctly against the message and the data will be accepted. If the data contains residual errors (which can only occur if the FEC mechanism was overwhelmed by the number of errors), the CRC will not match the packet and the data will be rejected. Because the radio link in this implementation is strictly one-way, rejected data is simply lost and there is no possibility of retransmission.

More preferably, the RF link utilizes a two-way (bi-directional) data transmission. By using a two-way data transmission, the data safety is significantly increased. By transmitting redundant information in the data emitted by the electrodes, the remote communication station is capable of recognizing errors and request a renewed transmission of the data. In the presence of excessive transmission problems such as, for example transmission over excessively great distances, or due to obstacles absorbing the signals, the remote communication station is capable of controlling the data transmission, or to manipulate on its own the data. With control of data transmission, it is also possible to control or re-set the parameters of the system, e.g., changing the transmission channel. This would be applicable for example if the signal transmitted is superimposed by other sources of interference, then by changing the channel the remote communication station could secure a flawless and interference free transmission. Another example would be if the signal transmitted is too weak, the remote communication station can transmit a command to increase its transmitting power. Still another example would be the remote communication station to change the data format for the transmission, e.g., in order to increase the redundant information in the data flow. Increased redundancy allows transmission errors to be detected and corrected more easily. In this way, safe data transmissions are possible even with the poorest transmission qualities. This technique opens in a simple way the possibility of reducing the transmission power requirements. This also reduces the energy requirements, thereby providing longer battery life. Another advantage of a two-way, bi-directional digital data transmission lies in the possibility of transmitting test codes in order to filter out external interferences such as, for example, refraction or scatter from the transmission current. In this way, it is possible to reconstruct falsely transmitted data.

Additionally, the external body motion sensor might include code, circuitry, and/or computational components to allow someone to secure (e.g., encrypt, password protect, scramble, etc.) the patient data communicated via wired and/or wireless connections. The code, circuitry, and/or computational components can be designed to match with other components in the system (e.g., camera, eye tracker, voice recorders, balance board, and/or CPU system) that can similarly include code, circuitry, and/or computational components to allow someone to secure (e.g., encrypt, password protect, scramble, etc.) the patient data communicated via wired and/or wireless connections.

In certain embodiments the motion analysis information related to the patient movement can be obtained by the same signal (e.g., electromagnetic) that could also be used to wirelessly to transmit information between the central computer(s), sensors, and/or network connected components of a system. Such as, for example, one could use a change(s) in a Wi-Fi signal to measure patient movement in a known location (whereby the Wi-Fi signal could also be used to transmit additional information from/to the motion analysis system).

### Additional Hardware

In certain embodiments, the motion analysis system 100 includes additional hardware so that additional data sets can be recorded and used in the assessment of a subject for a movement disorder. For example, in certain embodiments, the motion analysis system 100 includes a force plate 106. The subject 104 can stand on the force plate 106 while being asked to perform a task and the force plate 106 will acquire balance data, which can be transmitted through a wired or wireless connection to the CPU 103. An exemplary force plate is the Wii balance board (commercially available from Nintendo). Typically, the force plate will include one or more load sensors. Those sensors can be positioned on the bottom of each of the four legs of the force plate. The sensors work together to determine the position of a subject's center of gravity and to track their movements as they shift your weight from one part of the board to another. Each is a small strip of metal with a sensor, known as a strain gauge, attached to its surface. A gauge consists of a single, long electrical wire that is looped back and forth and mounted onto a hard surface, in this case, the strip of metal. Applying a force on the metal by standing on the plate will stretch or compress the wire. Because of the changes to length and diameter in the wire, its electrical resistance increases. The change in electrical resistance is converted into a change in voltage, and the sensors use this information to figure out how much pressure a subject applied to the plate, as well as the subject's weight.

The sensors' measurements will vary depending on a subject's position and orientation on the plate. For example, if a subject is standing in the front left corner, the sensor in that leg will record a higher load value than will the others. A microcomputer in the plate takes the ratio of the load values to the subject's body weight and the position of the center of gravity to determine the subject's exact motion. That information can then be transmitted to the CPU, through a wireless transmitter in the force plate (e.g., Bluetooth) or a wired connection. In certain embodiments, the individual data recorded from each individual sensor in the force plate can be sent individually to the CPU, or after being processed (in whole or part) within circuitry in the force plate system. In certain embodiments, the system can use digital and/or analog circuitry (such as for example a Wheatstone bridge) and/or systems such as those used in digital or analog scales.

The CPU 103 receives the data from the force plate and runs a load detecting program. The load detecting program causes the computer to execute a load value detecting step, a ratio calculating step, a position of the center of gravity calculating step, and a motion determining step. The load value detecting step detects load values put on the support board measured by the load sensor. The ratio calculating step calculates a ratio of the load values detected by the load detecting step to a body weight value of the player. The position of the center of gravity calculating step calculates a position of the center of gravity of the load values detected by the load detecting step. The motion determining step determines a motion performed on the support board by the player on the basis of the ratio and the position of the center of gravity. Alternatively, the force plate can include a processor that performs the above-described processing, which processed data is then transmitted to the CPU 103. Alternatively, in certain embodiments only one of the steps is performed and/or any combination of steps of the load detecting program.

An exemplary force plate and systems and methods for processing the data from the force plate are further described for example in U.S. pat. publ. no. 2009/0093305, the content of which is incorporated by reference herein in its entirety.

In other embodiments, the motion analysis system 100 includes an eye tracking device 107. FIG. 1 illustrates an exemplary set-up in which the eye tracking device is separate from the image capture device 101. However, in other embodiments, the eye tracking device 107 can be integrated into image capture device 101. Alternatively, a camera component of image capture device 101 can function as eye tracking device 107. A commercially available eye tracking device may be used. Exemplary such devices include ISCAN RK-464 (eye tracking camera commercially available from ISCAN, Inc., Woburn, Mass.), EYELINK II (eye tracking camera commercially available from SR Research Ltd., Ottawa, Canada) or EYELINK 1000 (eye tracking camera commercially available from SR Research Ltd., Ottawa, Canada), or Tobii T60, T120, or X120 (Tobii Technology AB, Danderyd, Sweden). The EYELINK 1000 (eye tracking camera commercially available from SR Research Ltd., Ottawa, Canada) is particularly attractive because subjects do not need to wear any head-mounted apparatus, which is often heavy and bothersome, particularly for young subjects, making tracker calibration a challenge with younger children. Eye-tracker calibration and raw-data processing (e.g., to extract saccades, eliminate blinks, etc.) may be carried out using known techniques. See, e.g., Chan, F., Armstrong, I. T., Pari, G., Riopelle, R. J., and Munoz, D. P. (2005) Saccadic eye movement tasks reveal deficits in automatic response inhibition in Parkinson's disease. Neuropsychologia 43:784-796; Green, C. R., Munoz, D. P., Nikkei, S. M., and Reynolds, J. N. (2007) Deficits in eye movement control in children with Fetal Alcohol Spectrum Disorders. Alcoholism: Clinical and Exp. Res. 31:500-511; Peltsch, A., Hoffman, A., Armstrong, I., Pari, G., and Munoz, D. P. (2008) Saccadic impairments in Huntington's disease correlate with disease severity. Exp. Brain Res. (in press); Peters, R. J., Iyer, A., Itti, L., & Koch, C. (2005) Components of bottom-up gaze allocation in natural images. Vision Research, 45(8), 2397-2416, or Tseng et al., U.S. Pat. No. 8,808,195, the content of each of which is incorporated by reference herein in its entirety. In certain embodiments, the camera system 105 can perform aspects of the eye tracking process.

In alternative embodiments, data can be recorded from sound sensors, such as for example voice data. Sound data, such as voice data can be analyzed in many ways, such as for example as a function of intensity, timing, frequency, waveform dynamics, and be correlated to other data recorded from the system. For example, patient data could analyze the power in specific frequency bands that correspond to sounds that are difficult to make during certain movement disorders. In alternative embodiments, the system could use voice recognition so that analysis could be completed by the CPU to determine if a patient could complete cognitive tasks, such as for example remembering words, or to make complex analogies between words. The processes associated with this data could be analog and/or digital (as could all processes throughout this document). In alternative embodiments, the sound sensors could be connected to at least one trigger in the system and/or used as a trigger. See methods examples in: “Digital Signal Processing for Audio Applications” by Anton Kamenov (December 2013); “Speech and Audio Signal Processing: Processing and Perception of Speech and Music” by Ben Gold, Nelson Morgan, Dan Ellis (August 2011); and “Small Signal Audio Design” by Douglas Self (January 2010), the content of each of which is incorporated by reference herein in its entirety. In alternative embodiments, data can be recorded from the eye, such as eye tracking sensors and/or electrooculogram systems. Eye data can be analyzed in many ways, such as for example eye movement characteristics (e.g., path, speed, direction, smoothness of movements), saccade characteristics, Nystagmus characteristics, blink rates, difference(s) between individual eyes, and/or examples such as those described in, and be correlated to other data recorded from the system. In alternative embodiments, the eye sensors could be connected to at least one trigger in the system and/or used as a trigger. In alternative embodiments, data can be recorded from alternative electrophysiological analysis/recording systems, such as for example EMG or EEG systems.

Other sensors can be implemented such as electrophysiology sensors (e.g., EMG, EEG, EKG, respiratory rates), and/or sensors capable of capturing metabolic and/or bio-functional signals (e.g., blood-oxygen level, Oxygen situation, respiratory rate, blood sugar, galvanic skin response).

### Synchronization

The individual component(s) (data acquisition measurement devices (e.g., accelerometer, camera, gyroscope) and/or CPU) of the system can be synchronized via any method known in the field, and communication can take place with wired and/or wireless connections with data that can be of any form, including digital and analog data, and be transmitted uni-directionally and/or bi-directionally (or multi-directionally with multiple components) in any fashion (e.g., serial and/or parallel, continuously and/or intermittently, etc.) during operation. For example, digital information of large data sets can be aligned by synchronizing the first sample and the interval between subsequent samples. Data communicated between at least two devices can be secured (e.g., encrypted), transmitted real-time, buffered, and/or stored locally or via connected media (such as for example for later analysis). In alternative embodiments, the individual components of the system can operate independently and be integrated at a later time point by analyzing the internal clocks of the individual components for offline synchronization. In alternative embodiments, different components and/or sets of components can be synchronized with different methods and/or timings. In certain embodiments, trigger information can be used to mark information about a subject and/or movements that are being assessed by the motion analysis system, such as for example marking when a set of movements of a task begin, and/or marking individual movements in a set of tasks (such as marking each step a patient takes).

Usually, two groups of signals will be used in synchronization: timing and triggering (although in certain embodiments, timing can be sufficient). Timing signals usually repeat in a defined, periodic manner and are used as clocks to determine when a single data operation should occur. Triggering signals are stimuli that initiate one or more component functions. Triggering signals are usually single events that are used to control the execution of multiple data operations. The system and/or components can use individual or multiple triggering and/or timing signals.

A variety of timing signals can be used in synchronization. In the simplest form, the individual components of the system run on the same clock(s) (or individual clocks that were synchronized prior to, during, and/or after data acquisition). In alternative methods, additional timing signals can be generated during certain operations of the system, these timing signals could be categorized based on the type of acquisition implemented. For an analog to digital input example, a sample clock in (or connected to) at least one of the data acquisition components of the system controls the time interval between samples, and each time the sample clock ticks (e.g., produces a pulse), one sample (per acquisition channel) is acquired. A Conversion Clock is a clock on or connected to the data acquisition components of the system that directly causes analog to digital conversion. The sampling interval is started on the sample clock tick and then the conversion clock is used to convert the analog signal coming out of a component like a multiplexer on the system with the conversion clock. For counter operations, a counter time-base could be generated from a clock connected to the components of the devices. Triggering signals can be used for numerous functions, such as for example: a start trigger to begin an operation; a pause trigger to pause an ongoing operation; a stop trigger to stop an ongoing operation; or a reference trigger to establish a reference point in an input operation (which could also be used to determine pre-trigger (before the reference) or post-trigger (after the reference) data). Counter output can also be set to re-triggerable so that the specific operation will occur every time a trigger is received. As described herein, event identification (e.g., a specific movement) can also be completed via a software based algorithm(s), such as to motion being analyzed, to serve as a synchronization and/or trigger point (e.g., the analyzed kinematic/kinetic signals of a hand in motion, such as opening and/or closing, could be used to identify the start of a movement and be used as a trigger signal for a task to be analyzed).

There are multiple types of synchronization that can be implemented in the system. In general, these types can be abstracted based on the physical level at which they occur. At the lowest level, multi-function synchronization occurs within a single component of the system. The next level is multi-component synchronization which occurs between data acquisition modules (and/or generation tasks (note, the different forms of synchronization can allow for two way communication of data, including both the reading and writing of information during the communication)). Finally, at the highest level multi-group synchronization occurs between groups of components in the system. Multi-group synchronization is the alignment of signals for multiple data acquisition tasks (or generation tasks). This can be accomplished on a single component by routing one signal to the circuitry of different functions, such as analog input, analog output, digital input/output (I/O) and counter/timer operations. Multi-group synchronization is important when trying to synchronize a small number of mixed-signals, such as for example analog data clocked with digital lines, PID control loops, or the frequency response of a system. Multi-component synchronization involves coordinating signals between components. Synchronization between components can use an external connection to share the common signal, but can allow for a high degree of accuracy between measurements on multiple devices. Multi-group synchronization allows multiple sets of components to share at least a single timing and/or triggering signal. This synchronization allows for the expansion of component groups into a single, coordinated structure. Multi-group synchronization can allow for measurements of different types to be synchronized and can be scaled for our system across numerous sets of components. At least one timing or trigger signal can be shared between multiple operations on the same device to ensure that the data is synchronized. These signals are shared by simple signal routing functions that enable built in connections. Further synchronization and communication between components of the system, can be made with any method known in the field, such as for example with methods such as those explained in Data Acquisition Systems: From Fundamentals to Applied Design by Maurizio Di Paolo Emilio (Mar. 22, 2013); Low-Power Wireless Sensor Networks: Protocols, Services and Applications (SpringerBriefs in Electrical and Computer Engineering) by Suhonen, J., Kohvakka, M., Kaseva, V., Hämäläinen, T. D., Hännikäinen, M. (2012); Networking Bible by Barrie Sosinsky (2009); Synchronization Design for Digital Systems (The Springer International Series in Engineering and Computer Science) by Teresa H. Meng (1990); and Virtual Bio-Instrumentation: Biomedical, Clinical, and Healthcare Applications in LabVIEW by Jon B. Olansen (December 2001) which are incorporated by reference herein in their entirety.

### Data Processing

The motion analysis system 100 includes a central processing unit (CPU) 103 with storage coupled to the CPU for storing instructions that when executed by the CPU cause the CPU to execute various functions. Initially, the CPU is caused to receive a first set of motion data from the image capture device related to at least one joint of a subject while the subject is performing a task and receive a second set of motion data from the external body motion sensor related to the at least one joint of the subject while the subject is performing the task. The first and second sets of motion data can be received to the CPU through a wired or wireless connection as discussed above. In certain embodiments, additional data sets are received to the CPU, such as balance data, eye tracking data, and/or voice data. That data can also be received to the CPU through a wired or wireless connection as discussed above.

There are any number of tasks that the subject can perform while being evaluated by the motion analysis system. Exemplary tasks include discrete flexion of a limb, discrete extension of a limb, continuous flexion of a limb, continuous extension of a limb, closing of a hand, opening of a hand, walking, rotation of a joint, holding a joint in a fixed posture (such as to assess tremor while maintaining posture), resting a joint (such as to assess tremor while resting), standing, walking, and/or any combination thereof. Tasks could also include movements which are performed during basic activities of daily living, such as for example walking, buttoning a shirt, lifting a glass, or washing oneself. Tasks could also include movements that are performed during instrumental activities of daily living, which for example could include motions performed during household cleaning or using a communication device. This list of tasks is only exemplary and not limiting, and the skilled artisan will appreciate that other tasks not mentioned here may be used with systems of the disclosure and that the task chosen will be chosen to allow for assessment and/or diagnosis of the movement disorder being studied. Analysis of the tasks can be made in real time and/or with data recorded by the system and analyzed after the tasks are completed.

For when two different sets of data are being recorded, the CPU includes software and/or hardware for synchronizing data acquisition, such as using methods described above. For example, software on the CPU, can initiate the communication with an image capture device and at least one external patient worn motion sensor. Once the individual components establish a connection (such as for example via a standard handshaking protocol and/or other methods described above), data from all or some of the device components can be recorded in a synchronized manner, and/or stored and/or analyzed by the CPU. The operator can choose to save all or just part of the data as part of the operation. The operator (and/or patient) can initiate a motion analysis system to track a certain task (such as flexing a joint). The initiation (and/or conclusion) of the task can be marked (such as for example by a device which provides a trigger, such as user operated remote control or keyboard, or automatically via software based initiation) on the data that is being recorded by the CPU (and/or in all or some of the individual system components (e.g., an external patient worn motion sensor)) such as could be used for analysis. The data being recorded can be displayed on a computer screen during the task (and/or communicated via other methods, such as for example through speakers if an audio data is being assessed). The data may be stored and analyzed later. The data may be analyzed in real-time, in part or in full, and the results may be provided to the operator and or stored in one of the system components. The data and analysis results could be communicated through wired or wireless methods to clinicians who can evaluate the data, such as example remotely through telemedicine procedures (additionally in certain embodiments the system can be controlled remotely). The process could be run in part or entirely by a patient and/or another operator (such as for example a clinician). In an alternative embodiment, all of the components of the system can be worn, including the image capturing camera, to provide a completely mobile system (the CPU for analysis could be housed on the patient, or the synchronized data could be communicated to an external CPU for all or part of the analysis of the data).

As mentioned above, the system can obtain data from 1 or more joints, e.g., 1 joint, 2 joints, 3 joints, 4 joints, 5 joints, 6 joints, 7 joints, 8 joints, 9 joints, 10 joints, 15 joints, 20 joints, or more joints. In certain embodiments, data are recorded with all the sensors, and only the data recorded with the sensors of interest are analyzed. In other embodiments, only data of selected sensors is recorded and analyzed.

The CPU and/or other components of the system are operably linked to at least one trigger, such as those explained above. In alternative embodiments, a separate external component (or an additional integrated system component) can be used to trigger all or some of the components of the system (such as for example one or more remotes of a Wii video gaming system, commercially available from Nintendo, an external computer, a tablet device, an external keyboard, a watch, and/or a mobile phone). In alternative embodiments, the trigger could be voice activated, such as when using a microphone. In alternative embodiments the trigger could be motion activated (such as for example through hand movements, body postures, and/or specific gestures that are recognized). A trigger can mark events into the recorded data, in an online fashion. Additionally, any one of these external devices can be used to write to the data being recorded to indicate when a task is being performed by an individual being evaluated with the system (for example an observer, or individual running the system, while evaluating a patient can indicate when the patient is performing one of the tasks, such as using the device to mark when a flexion and extension task is started and stopped). In certain embodiments the events marked by a trigger can later be used for further data analysis, such as calculating duration of specific movements, or for enabling additional processes such as initiating or directing brain stimulation. In certain embodiments, multiple triggers can be used for functions that are separate or integrated at least in part.

The CPU is then caused to calculate kinematic and/or kinetic information about at least one joint of a subject from a combination of the first and second sets of motion data, which is described in more detail below. Then the CPU is caused to output the kinematic and/or kinetic information for purposes of assessing a movement disorder. Exemplary movement disorders include diseases which affect a person's control or generation of movement, whether at the site of a joint (e.g., direct trauma to a joint where damage to the joint impacts movement), in neural or muscle/skeletal circuits (such as parts of the basal ganglia in Parkinson's Disease), or in both (such as in a certain pain syndrome chronic pain syndrome where for instance a joint could be damaged, generating pain signals, that in turn are associated with change in neural activity caused by the pain). Exemplary movement disorders include Parkinson's disease, Parkinsonism (aka., Parkinsonianism which includes Parkinson's Plus disorders such as Progressive Supranuclear Palsy, Multiple Systems Atrophy, and/or Corticobasal syndrome and/or Cortical-basal ganglionic degeneration), tauopathies, synucleinopathies, Dementia with Lewy bodies, Dystonia, Cerebral Palsy, Bradykinesia, Chorea, Huntington's Disease, Ataxia, Tremor, Essential Tremor, Myoclonus, tics, Tourette Syndrome, Restless Leg Syndrome, Stiff Person Syndrome, arthritic disorders, stroke, neurodegenerative disorders, upper motor neuron disorders, lower motor neuron disorders, muscle disorders, pain disorders, Multiple Sclerosis, Amyotrophic Lateral Sclerosis, Spinal Cord Injury, Traumatic Brain Injury, Spasticity, Chronic Pain Syndrome, Phantom Limb Pain, Pain Disorders, neuropathies, Metabolic Disorders and/or traumatic injuries.

The data can be used for numerous different types of assessments. In one embodiment, the data is used to assess the effectiveness of a stimulation protocol. In such embodiments, a subject is evaluated with the motion analysis system at a first point in time, which services as the baseline measurement. That first point in time can be prior to receiving any stimulation or at some point after a stimulation protocol has been initiated. The CPU is caused to calculate a first set of kinematic and/or kinetic information about the at least one joint of a subject from a combination of the first and second sets of motion data while the subject is performing a task. That data is stored by the CPU or outputted for storage elsewhere. That first set of kinematic and/or kinetic information is the baseline measurement. The subject is then evaluated with the motion analysis system at a second point in time after having received at least a portion or all of a stimulation protocol. The CPU is caused to calculate a second set kinematic and/or kinetic information about the at least one joint of a subject from a combination of the first and second sets of motion data while the subject is performing a task. That second set of data is stored by the CPU or outputted for storage and/or presentation elsewhere. The first and second sets of data are then compared, either by the CPU or by a physician having received from the CPU the outputted first and second sets of data. The difference, if any, between the first and second sets of data informs a physician as to the effectiveness of the stimulation protocol for that subject. This type of monitoring can be repeated numerous times (i.e., more than just a second time) to continuously monitor the progress of a subject and their response to the stimulation protocol. The data also allows a physician to adjust the stimulation protocol to be more effective for a subject.

In other embodiments, the motion analysis system of the disclosure is used for initial diagnosis or assessment of a subject for a movement disorder. Such embodiments, use a reference set of data to which a subject is compared in order to make a diagnosis or assessment of the subject. The reference set, stored on the CPU or remotely on a server operably coupled to the CPU, includes data of normal healthy individuals and/or individuals with various ailments of various ages, genders, and/or body type (e.g., height, weight, percent body fat, etc.). Those healthy individuals and/or individuals with various ailments have been analyzed using the motion analysis system of the disclosure and their data is recorded as baseline data for the reference data set (in alternative embodiments, a reference set can be developed by modeling simulation motion data and/or a reference set could be developed from a model developed based on the analysis of assessments of healthy individuals and/or patients). The reference set of data could be based on previous measurements taken from the patient currently being assessed. A test subject is then evaluated using the motion analysis system of the disclosure and their kinematic and/or kinetic information is compared against the appropriate population in the reference set, e.g., the test subject data is matched to the data of a population within the reference set having the same or similar age, gender, and body type as that of the subject. The difference, if any, between the test subject's kinematic and/or kinetic information as compared to that of the reference data set allows for the assessment and/or diagnosis of a movement disorder in the subject. Typically, at least a 25% difference (e.g., 30%, 35%, 40%, 45%, 50%, 60%, 70%, 80%, 90%, 95%, 95%, or 99%) between the kinematic and/or kinetic information of the subject and that of the reference data set is an indication that the subject has a movement disorder. The greater the difference between the kinematic and/or kinetic information of the subject and that of the reference data set allows for the assessment of the severity or degree of progression of the movement disorder. For example, a subject with at least 50% difference (e.g., 55%, 60%, 70%, 80%, 90%, 95%, 95%, or 99%) between their kinematic and/or kinetic information and that of the reference set would be considered to have a severe form of the movement disorder. In certain diagnostic tests, just the presence of a characteristic, for example a Babinski sign, would be enough to demonstrate a patient has an ailment, which for a Babinski would demonstrate an upper motor neuron deficit. In certain diagnostic evaluations, smaller differences can be demonstrated and/or tracked, such as for example to track a patients progress to a therapy (such as when comparing a patient's motion analysis results previous motion analysis results from a previous exam of the patient). Furthermore multiple small differences (e.g., less than 25%), for example in at least two criteria, between a tested patient and a data set can be used to make a probabilistic diagnosis that a patient suffers from a disorder (for example, in certain alternative embodiments, multiple changes, with changes as small as 1%, could be used to make a statistical model, that can have a predictive capabilities with high probability that a disease is present (such as for example with 80%, 90%, 95%, 99%,99.9% and 100% probability)-for example: a statistical model based on 10 different movement task characteristics could be assessed which makes a diagnosis based on a weighted probabilistic model, a disease diagnosis model based on derived results or grouped results (e.g., positive presence of a Babinski sign when 99 other tested criteria were not met, would still be an indication of an upper motor neuron disease), and/or model based on patient history and a result(s) derived from the motion analysis system while patients are performing a movement or set of movement tasks).

Additionally, measures could be used to determine characteristics of movement (such as quality and/or kinematics) at a baseline visit and used to evaluate the impact of a therapy throughout the course a treatment paradigm (eventually the system could be integrated into the therapy providing system to make a closed loop system to help determine or control therapeutic dosing, such as integrating a motion analysis suite with a neurostimulation system (which could further be integrated with other systems, such as computerized neuro-navigation systems and stimulation dose models such as could be developed with finite element models, see for example U.S. pat. publ. nos. 2011/0275927 and 2012/0226200, the content of each of which is incorporated by reference herein in its entirety)). A motion analysis suite could further be used to develop new clinical scores based on the quantitative information gathered while evaluating patients (such as for example, one could track Parkinson patients with the system and use the results to come up with a new clinical metric(s) to supplement the UPDRS part III scores for evaluating the movement pathology in the patient).

Certain exemplary embodiments are described below to illustrate the kinematic and/or kinetic information that is calculated for the first and second data sets and the output of that calculation.

In one embodiment, bradykinesia is assessed. A subject is asked to perform 10 arm flexion-extension movements as fast as possible (note that this number (e.g., 10 movements) is just exemplary, and that 1, 2, 3 movements, and so on could be completed. Furthermore, in alternative embodiments, just one movement type (e.g., flexion), any other type of movement(s), and/or groups of movement can be examined. Additionally, any joint or group of joints can be assessed. Furthermore, in certain embodiments the tests could be completed with more or less body motion sensors placed at different locations on the body, and/or the analysis can be completed for different joint(s)). Furthermore, during some tests the patient will perform more or less movements than asked (for example, sometimes they are unable to complete all the tasks due to a pathology, other times they might simply loose count of how many movements they have performed). This test can then be repeated with both arms (simultaneously or independently) or conducted with a single arm. The image capture device records and transmits these movements of the subject to the CPU, which becomes the first set of motion data. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger to at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offset, last value greater than 0, for example on a trigger data channel). Onset, offset, and total duration are displayed to the user, who can edit them if needed. Alternatively, in certain embodiments the trigger data could be automatically obtained from the motion data.

For this task, the image capture device records and transmits wrist joint position data (X, Y, Z) related to the 10 flexion-extension movements (ideally 20 movements total, 10 flexion and 10 extension movements). Those movements are filtered with a low-pass filter (cut-off 10 Hz, 11 coefficients) designed with the frequency sampling-based finite impulse response (FIR) filter design method. Then filtered X, Y, Z components are differentiated with a central difference algorithm to obtain velocities Vx, Vy, and Vz. Then, speed profiles are calculated as v=sqrt(Vx.\hat{ }2+Vy.\hat{ }2+Vz.\hat{ }2). Speed profiles are finally segmented (onset and offset are identified) to extract the 20 movements. To determine onset and offset of each of the 20 movements, X, Y, and Z are filtered with a FIR low-pass filter (cut-off 0.01 Hz, 11 coefficients); then velocities 1_Vx, 1_Vy, and 1_Vz are calculated by differentiating the filtered X, Y, and Z respectively, and speed profiles are calculated as v_1=sqrt(1_Vx.\hat{ }2+1_Vy.\hat{ }2+1_Vz.\hat{ }2). Peaks of (−v_1) are extracted automatically. This step identifies minimum values of v_1, which are assumed to be the same as minimum values of v (and easier to extract as the signal is less noisy). These points are used to define onset and offset of the single 20 movements. In this analysis and/or other embodiments of segmentation process, other methods can be used for extracting onset and offset values. For example, a method based on thresholding speed or velocity profiles or a method based on zero crossings of position data or velocity components or a combination of the above, etc. could be used. Results of segmentation are displayed to the user who can edit them if needed. Segmentation of the movements can be confirmed by the data from the external body sensor. Ideally, both information from the image capture and external body sensor components is used together for the segmentation process (see below). For this task, at least one accelerometer (and or gyroscope) can also be mounted on the subject's index finger, wrist, or comparable joint location (e.g., a joint location which correlates with the movement being performed). During this task, the accelerometer data is processed with a 4^{th} order low-pass Butterworth filter with a cut-off frequency of 5 Hz. In other embodiments of analysis other types of filters designed with any method known in the art can be used, such as for example Window-based FIR filter design, Parks-McClellan optimal FIR filter design, infinite impulse response (IIR) filter, Butterworth filter, Savitzky-Golay filter, etc. Additionally, filters with different parameters and characteristics (coefficients/order, cut-off frequency, bandwidth, impulse response, step response, etc.) can be used. Analog filters and/or analog methods may be used where appropriate. Similarly, differentiation can be performed using different algorithms, such as forward differencing, backward differencing, etc.

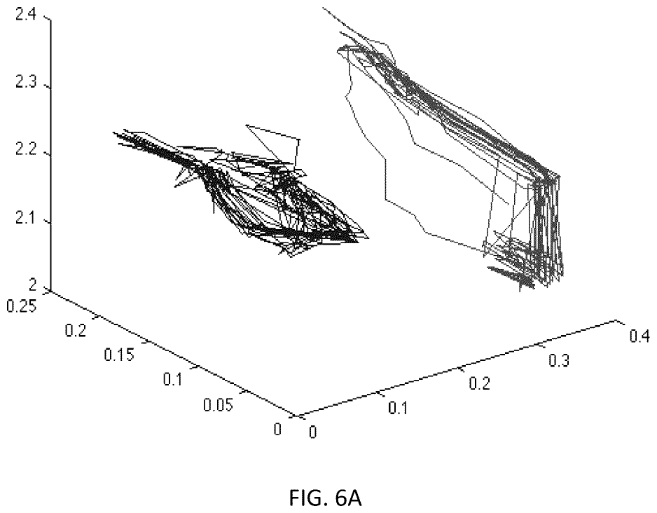

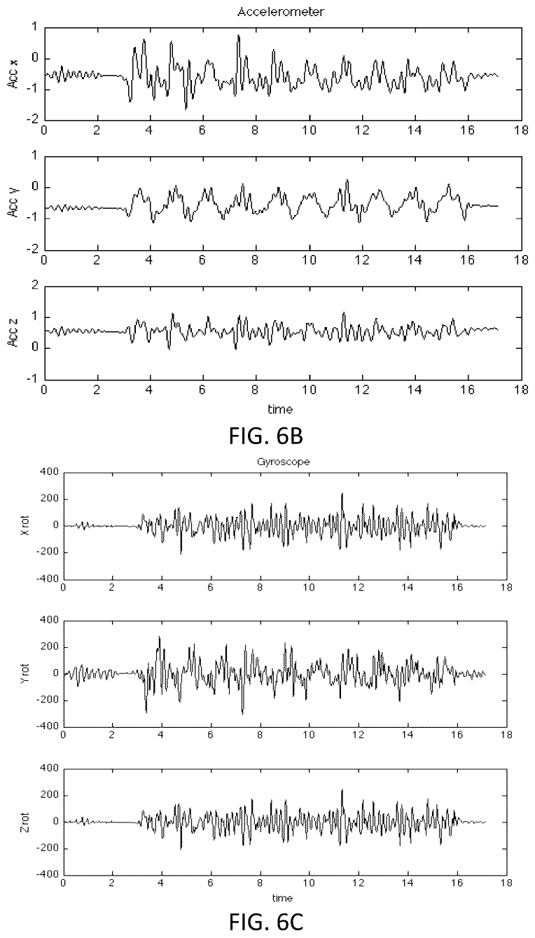

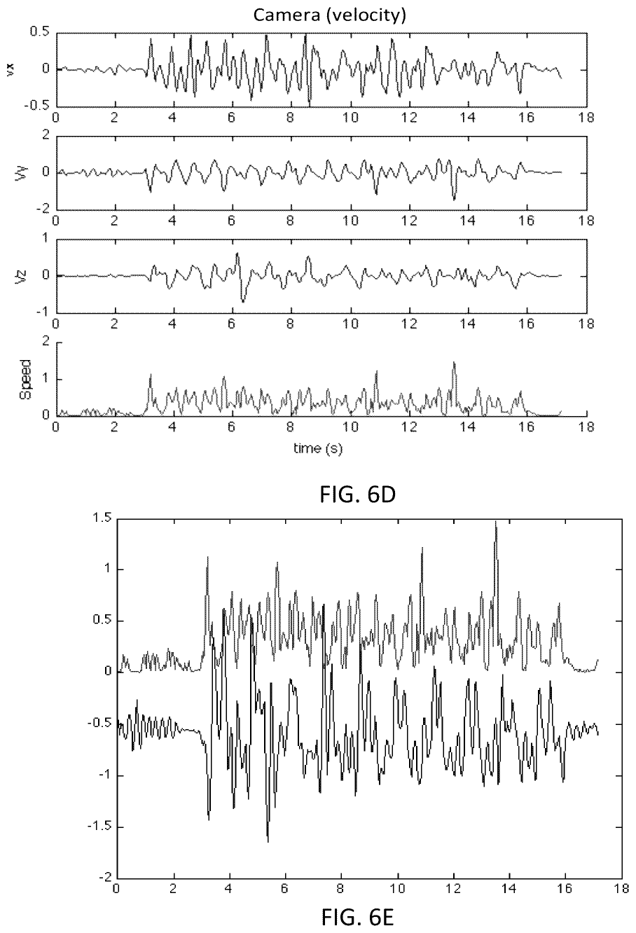

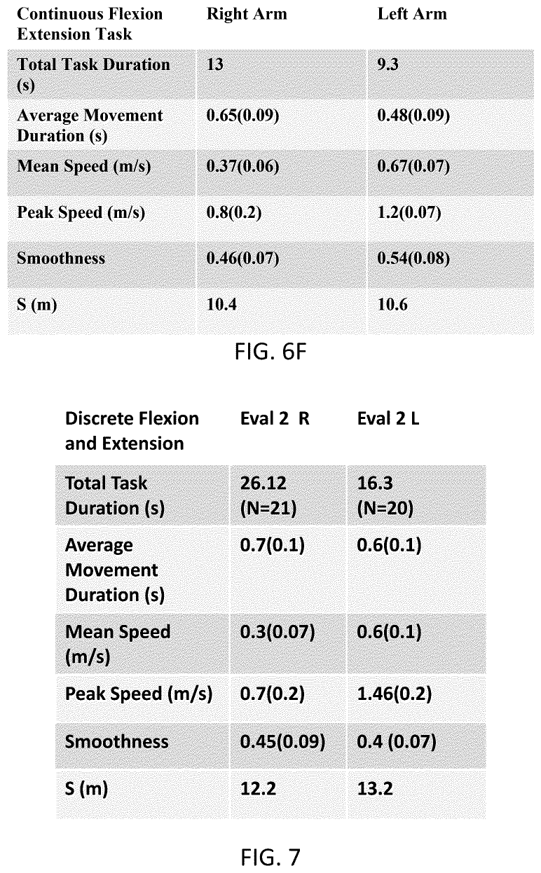

An example of this data and analysis for this task is shown in FIGS. 6A-6E. In FIG. 6A, a figure of position data recorded from the camera device indicating the position of the wrist in space, provided in X, Y, Z coordinates in the space of the subject, in the units of meters, during a test is provided. The blue line corresponds to the right wrist and the red line to the left wrist-note for tasks can be performed separately or together (this example data is for when the tasks for the left and right arm were performed individually, but demonstrated here on the same graph). In FIG. 6B, we provide the information from the accelerometers, provided in the X, Y, Z coordinates in the space relative to the accelerometer (i.e., relative to the measurement device) in relative units of the accelerometer-this data is for the right wrist. In FIG. 6C, we provide the information from the gyroscope in relative units of the gyroscope-this data is for the right wrist. In FIG. 6D, we provide the information of the velocity of movement in provided in X, Y, Z coordinates in the space of the subject, with the units of m/s, calculated based on the camera data—this data is for the right wrist. In FIG. 6E, we provide the information of the velocity (red line) based on the camera information in line with the data simultaneously recorded with the accelerometer (blue line)-note we provide the information in terms of m/s for the red line, and the blue line is given in terms of the relative units of the accelerometer-this data is for the right wrist (note the displayed Y-axis scale is in velocity and the x-axis is in units of time and the axis for the accelerometer is not shown as it is given in relative units). Here the accelerometer data was used to confirm the movements recorded with the camera (and vice versa), such that where one data set can be used to validate the other. The two sets of data can further be used such that the X, Y, Z acceleration information that was developed in the coordinate system of the accelerometer could be correlated to the X, Y, Z space of the patient's coordinate system.

The following metrics are extracted from the 20 segments of v, whose onset and offset is described above: movement mean speed (mean value of speed), movement peak speed (peak value of speed), movement duration: difference between offset of movement and onset of movement, movement smoothness (smoothness is a measure of movement quality that can be calculated as mean speed/peak speed; in this analysis and/or other embodiments smoothness can also be calculated as the number of speed peaks, the proportion of time that movement speed exceeds a given percentage of peak speed, the ratio of the area under the speed curve to the area under a similarly scaled, single-peaked speed profile, etc. Smoothness can also describe a general movement quality). Also calculated is the path length of the trajectory of the wrist joint (distance traveled in 3D space). See FIG. 6F.

The following metrics are the final output (kinematic and/or kinetic information) for this test: total duration of test; number of movements performed; movement mean speed (mean and standard deviation across all movements); movement peak speed (mean and standard deviation across all movements; movement duration (mean and standard deviation across all movements); movement smoothness (mean and standard deviation across all movements); and path length. In this analysis and/or other embodiments of the final output other statistical measures can be used such as for example variance, skewness, kurtosis, and/or high-order-moments. That data is then compared against the reference set or against the subject's prior set of kinematic and/or kinetic information for assessment of a movement disorder or assessment of the effectiveness of stimulation on treating the movement disorder.

In other embodiments, different computational methods and/or computational step order can be followed to determine kinematic and/or kinetic information output from the system. In certain embodiments, additional results that could be assessed include: acceleration (velocity, position, power, and/or other derived metrics (e.g., jerk)) as a function of joint(s) analyzed; acceleration (velocity, position, power, and/or other derived metrics (such as average, median, and/or standard deviation of these metrics)) as a function of movement(s) analyzed; acceleration (velocity, position, power, and/or other derived metrics) as a function of joint(s) position(s) analyzed; trajectory information (direction, quality, and/or other derived metrics) as a function of joint(s), movement(s), and/or joint(s) position(s); timing data related to movements (e.g., time to fatigue, time to change in frequency of power, time of task, time component of a task, time in position); joint or group joint data (absolution position, relative position, dimensions, velocity, position, power, time in position, and/or other derived metrics); and/or analysis based on individual or combined elements of these examples.

While in the above example, we provided a method where the relative accelerometer data was integrated with the camera image data to aid in the segmentation of movement data captured by the camera (see example FIG. 6) and to confirm the task movement was completed as directed, the two components' information can be integrated to provide further correlated information about movement that would not be captured by either device independently. For example, in a certain embodiment the power frequency spectrum of acceleration during movement of a specific joint as recorded by the accelerometer can be analyzed as a function of the movement recorded with the image device (or vice versa). As another example, in a certain embodiment the information from the camera position information can be used to determine constants of integration in assessing the information derived from the accelerometer which require an integration step(s) (e.g., velocity). As another example, an accelerometer on its own provides acceleration data relative to its own body (i.e., not in the same fixed coordinate system of a subject being analyzed with the system), and a camera cannot always provide all information about a joints information during complicated movements due its field of view being obscured by a subject performing complicated movement tasks, and by bringing the data together from the two components of the system the loss of information from either can be filled-in by the information provided by the correlated information between the two components. As another example, in a certain embodiment the camera image recordings can be used to correct drift in motion sensors (such as drift in an accelerometer or gyroscope). As another example, in a certain embodiment the camera image recordings can be used to register the placement and movement of the accelerometer (or other motion analysis sensor) in a fixed coordinate system (accelerometers X, Y, and Z recording/evaluation axes move with the device). In another example, in a certain embodiment the accelerometer (or other motion analysis sensor) can be placed on body locations that can be obscured from the recording view of the camera system during body movements. In another example, in a certain embodiment the camera information can be used to remove the effects of gravity on the accelerometer recordings (by being able to determine relative joint and accelerometer position during movement, and thus the relative accelerometer axis to a true coordinate space the subject is in and thus localize the direction of gravity). In another example, in a certain embodiment the acceleration data from the accelerometer (such as for example total acceleration) could be correlated and analyzed as a function of specific characteristics in movement determined from the camera component (such as for example an individual and/or a group of joints' position, movement direction, velocity). In another example, in a certain embodiment gyroscopic data and accelerometer data can be transformed into data in a patient's fixed reference frame by co-registering the data with the video image data captured by the camera and used to correct for drift in the motion sensors while simultaneously and/or allowing for the determination of information not captured by the camera system, such as for example when a patients' movements obscure a complete view of the patient and joints from the camera. In certain situations, a camera alone could suffer from certain disadvantages (for example an occlusion of views, software complexity for certain joints (e.g., hand and individual fingers), sensitivity to lighting conditions) but these advantages can be overcome by coupling the system with a motion sensors); while motion sensors (such as accelerometers and/or gyroscopes) alone suffer from certain disadvantages (for example drift and a lack of a fixed coordinate system) which can be overcome by coupling the system with camera, for tasks and analysis that are important to the diagnosis, assessment, and following of movement disorders. In addition to these examples, more examples and alternative embodiments of the system follow describing the use of multiple motion analysis sensors (such as accelerometers, gyroscopes, and/or force plates), camera components, and/or other patient sensors (voice sensors, eye sensors, etc.) in various groups and combinations for the diagnosis, assessment, and following of movement disorders. These examples are only exemplary and not limiting, and the skilled artisan will appreciate that other methods of combining multiple components not mentioned here may be used with systems of the disclosure and that the components and task will be chosen to allow for assessment and/or diagnosis of the movement disorder being studied.

In another embodiment for assessing bradykinesia, a subject is asked to perform 10 arm flexion-extension movements (note that this number, joint, motion sensor(s) placement(s), and joint task is just exemplary). After each flexion or extension movement, the subject is asked to stop. The movements are performed as fast as possible. This test can then be repeated with both arms. The image capture device records and transmits these movements of the subject to the CPU, which becomes the first set of motion data. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger at the initiation of the task and asked to use the trigger at the completion of the task. In that manner, the trigger marks into the recorded data the start and end times for the task, which can be used to calculate the total duration of the experiment. In certain embodiments, the trigger could mark a single event, a part of an event, and/or multiple events or parts of event, such as all 10 flexion movements. The onset and offset of the trigger are calculated automatically by the CPU (Onset: first value greater than 0; Offset, last value greater than 0). Onset, offset, and total duration are displayed to the user, who can edit them if needed. Alternatively, in certain embodiments the trigger data could be automatically obtained from the motion data.

For this task, the image capture device records and transmits wrist joint position data (X, Y, Z) related to the 10 flexion-extension movements (ideally 20 movements total 10 flexion and 10 extension movements). Similarly, the accelerometer and optionally gyroscope are positioned on the wrist joint.

The data are analyzed similarly to above, but segmentation of speed profiles is performed differently such that the accelerometer (+gyroscope) data are scaled to be same length as the image capture data and the process of segmentation to extract the 20 single movements uses gyroscope data. Specifically, the Z component of the data recorded from the gyroscope is analyzed to extract peaks; starting at the time instant corresponding to each identified peak, the recording is scanned backward (left) and forward (right) to find the time instants where the Z component reaches 5% of the peak value (note in alternative embodiments other thresholds could be used (For example, such as 2%, 3%, 4%, 10%, 15% of peak value, depending on the signal-to-noise ratio.)). The time instants at the left and right are identified respectively as the onset and offset of the single movement (corresponding to the identified peak). This segmentation process leads to extraction of 10 movements. A similar process is repeated for the-Z component of the data recorded from the gyroscope to identify the remaining 10 movements.

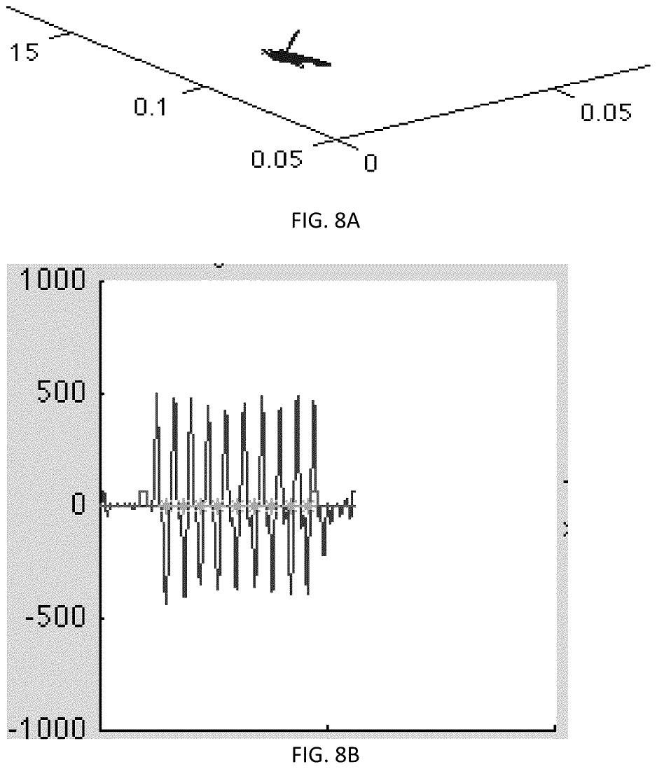

The following metrics are extracted from the 20 segments of v, whose onset and offset is described above: movement mean speed (mean value of speed), movement peak speed (peak value of speed), movement duration (difference between offset of movement and onset of movement), movement smoothness (mean speed/peak speed). Also calculated is the path length of the trajectory of the wrist joint (distance traveled in 3D space). Following a process similar to above, detailed in FIG. 6A-E, the data in FIG. 7, was determined.

The following metrics are the final output (kinematic and/or kinetic information) for this test: total duration of test; number of movements performed; movement mean speed (mean and standard deviation across all movements); movement peak speed (mean and standard deviation across all movements; movement duration (mean and standard deviation across all movements); movement smoothness (mean and standard deviation across all movements); and path length. That data is then compared against the reference set or against the subject's prior set of kinematic and/or kinetic information for assessment of a movement disorder or assessment of the effectiveness of stimulation on treating the movement disorder. As above, in other embodiments, different computational methods and/or step order can be followed to determine kinematic and/or kinetic information output from the system.

In still another embodiment, a subject is asked to perform 10 hand opening and closing movements, as fast as possible, while the hand is positioned at a fixed location (here for example the shoulder)—note that this number, joint, motion sensor(s) placement(s), and joint task is just exemplary. The image capture device records and transmits these movements of the subject to the CPU, which becomes the first set of motion data. This camera data can be used to assess if the patient is keeping their hand in a fixed location, for example by analyzing wrist or arm positions. Or in alternative embodiments, the camera data can be used to determine individual characteristics of the hand motion (such as for example individual finger positions) when assessed in conjunction with the accelerometer. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger to at the initiation of the task and asked to use the trigger at the completion of the task (herein the last evaluated hand open-closing task). In that manner, the trigger marks into the recorded data the start and end times for the task, which can be used to calculate the total duration of the experiment. The onset and offset of the trigger are calculated automatically by the CPU (e.g., Onset: first value greater than 0; Offset, last value greater than 0). Onset, offset, and total duration are displayed to the user, who can edit them if needed.

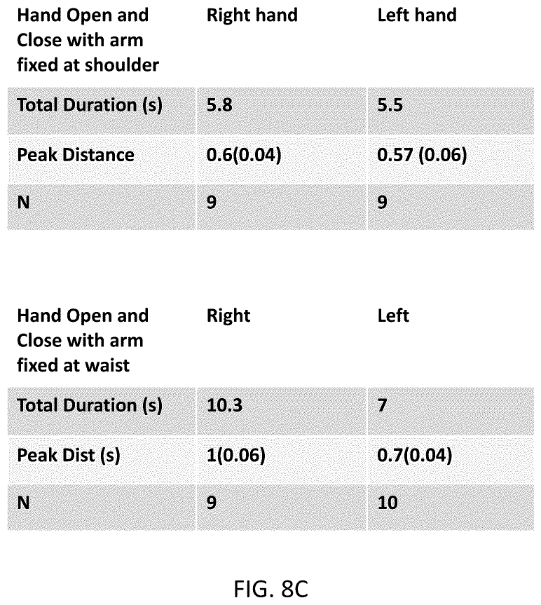

For this task, the image capture device records and transmits wrist joint position data (X, Y, Z) in a fixed coordinate space related to the opening and closing of the hand. Or alternatively, the image capture device is used to validate the position of the wrist and arm, and thus that the hand is fixed at the location chosen for the movement task evaluation (for example here at the shoulder), see FIG. 8A depicting wrist position. In FIG. 8A, the position of the hand is gathered from the data, as can be noticed compared to FIG. 6A the patient was able to follow the instructions of keeping the hand stable, as the limited movement was determined within the normal range in the case of the patient (e.g., the patient did not demonstrate the same range of movement depicted in the flexion and extension movement), and at point in X, Y, Z space of the patient that corresponds to the appropriate anatomical level (e.g., shoulder). In alternative tasks, the relative hand position can be tracked with a camera, and be used to determine what effect the location of the hand has on the hand open and closing speeds as determined with accelerometer and/or gyroscope data (see below). With a single camera alone it would not be possible to study all aspects of individual fingers as the hand closes (such as for example due to occlusion of views), yet accelerometer and gyroscopic data can fill this void; furthermore, the gyroscope and accelerometer cannot provide fixed joint position information (as the observation axes are dependent on the position of the recording systems); the combined information is particularly important for the diagnosis, evaluation, and following of movement disorders.

Similarly, the accelerometer and optionally gyroscope are positioned on the subject's index finger. Gyroscopic and acceleration data of the index finger is recorded. For example, in FIG. 8B, peaks of the rotational component of the gyroscope along its X axis is identified and displayed to the user (blue line in units of the gyroscopic device), the red lines show the triggering device, and the green line demonstrates the peak locations of the movements. The gyroscopic information, corresponding to the waveform characteristics of the data could be used to determine the time point when the hand was opened or closed (based on the rotational velocity approaching zero at this point). The distance between consecutive peaks (a measure of the time between two consecutive hand closing/opening movements) is calculated. The number of movements performed is calculated as the number of peaks +1. See FIG. 8C (top half for data gathered with the hand held at the shoulder). In FIG. 8C (bottom half), this same data is provided for the hand held at the waist, as confirmed by the camera system in a fixed coordinate space. The difference in hand speeds in these positions can only be confirmed through the use of data from both the image capture device and the external body sensors.

The following metrics are the final output for this test: total duration of test; number of movements performed; and time between two consecutive hand closing/opening movements (mean and standard deviation across all movements). That data is then compared against the reference set or against the subject's prior set of kinematic and/or kinetic information for assessment of a movement disorder or assessment of the effectiveness of stimulation on treating the movement disorder. As above, in other embodiments, different computational methods and/or step order can be followed to determine kinematic and/or kinetic information output from the system.

In still another embodiment, a subject is asked to perform combined movements (flexion followed by hand opening/closing followed by extension followed by hand opening/closing) 10 times as fast as possible (note that this number, joint, motion sensor(s) placement(s), and joint task is just exemplary).

The image capture device records and transmits these movements of the subject to the CPU, which becomes the first set of motion data. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offset, last value greater than 0). Onset, offset, and total duration are displayed to the user, who can edit them if needed.

The final output is total duration of test, and a combination of the above data described in the individual tests. That data is then compared against the reference set or against the subject's prior set of kinematic and/or kinetic information for assessment of a movement disorder or assessment of the effectiveness of stimulation on treating the movement disorder. In alternative tasks, more complicated movements can be performed where the movements are occurring simultaneously. As above, in other embodiments, different computational methods and/or step order can be followed to determine kinematic and/or kinetic information output from the system.

In still another embodiment, a subject is asked to touch their nose with their index finger, as completely as possible, 5 times (note that this number, joint, motion sensor(s) placement(s), and joint task is just exemplary). The image capture device records and transmits these movements of the subject to the CPU, which becomes the first set of motion data. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger to at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offset, last value greater than 0). Onset, offset, and total duration are displayed to the user, who can edit them if needed.

For this task, the image capture device records and transmits wrist joint position data (X, Y, Z). The accelerometer and optionally gyroscope are positioned on the subject's index finger.

For this task, the image capture device records and transmits wrist joint position data (X, Y, Z). Those movements are filtered with a FIR low-pass filter (cut-off 10 Hz, 11 coefficients). Then filtered X, Y, Z are differentiated to obtain velocities Vx, Vy, and Vz. Then, speed profiles are calculated as v=sqrt(Vx.\hat{ }2+Vy.\hat{ }2+Vz.\hat{ }2). Speed profiles are finally segmented (onset and offset are identified) to extract the 20 movements. To determine onset and offset of each of the 20 movements, X, Y, and Z are filtered with a FIR low-pass filter (cut-off 0.01 Hz, 10 coefficients); then velocities 1_Vx, 1_Vy, and 1_Vz are calculated by differentiating the filtered X, Y, and Z respectively, and speed profiles are calculated as v_1=sqrt(1_Vx.\hat{ }2+1_Vy.\hat{ }2+1_Vz.\hat{ }2). Peaks of (−v_1) are extracted automatically. This step identifies minimum values of v_1, which are assumed to be the same as minimum values of v (but easier to extract as the signal is less noisy). These points are used to define onset and offset of the single 20 movements. Results of segmentation are displayed to the user who can edit them if needed.

Acceleration is calculated as root square of Acc_X, Acc_Y, and Acc_Z, which are recorded with the accelerometer. Signal is de-trended (mean is removed), FFT magnitude is calculated (N=1024), and values between 6 and 9 Hz (as well as between 6 and 11 Hz) are summed. The resulting value represents the power of the signal in the range 6-9 Hz (or 6-11 Hz). For this example, tremor is calculated as the power of the signal in the range 6-9 Hz (or 6-11 Hz) divided by the total power of the signal.

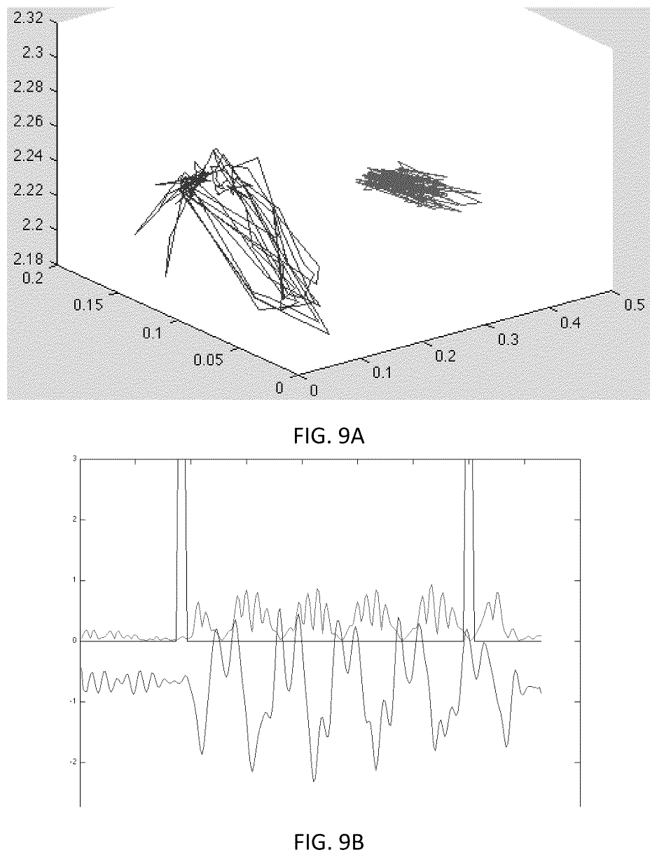

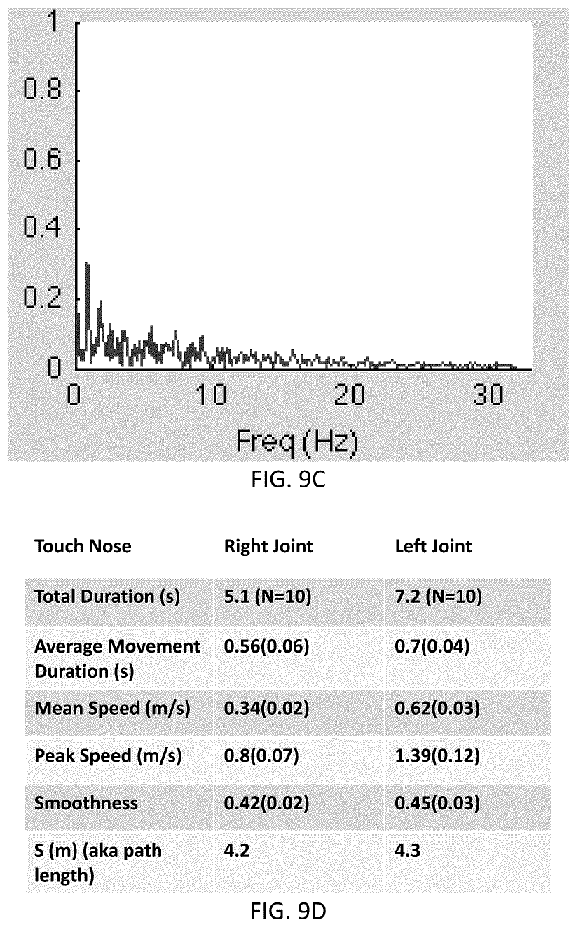

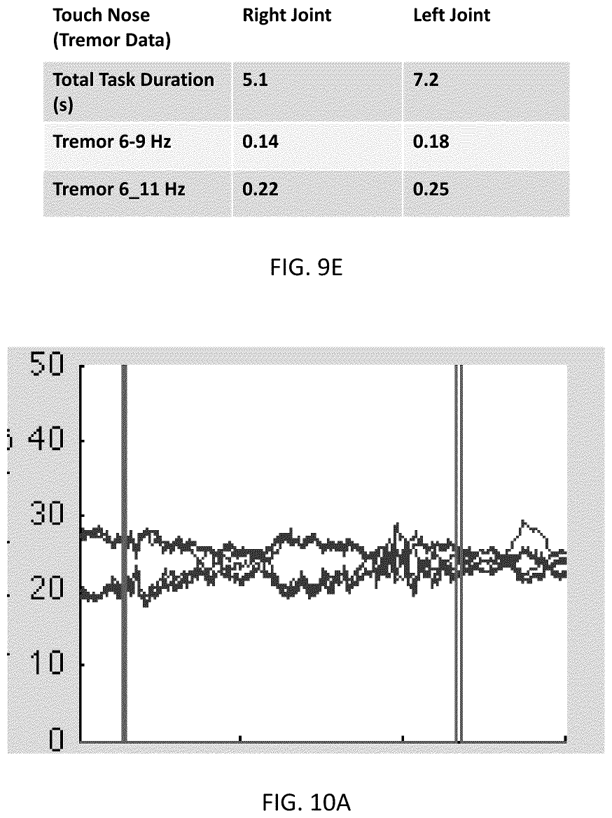

In FIG. 9A, we show an example of position data recorded by the camera provided in X, Y, Z coordinates in the space of the subject, in the units of meters, during the test. The blue line corresponds to the right wrist and the red line to the left wrist-note for tasks can be performed separately or together (this example data is for when the tasks for the left and right arm were performed individually). In FIG. 9B, we show velocity determined from the camera data (red), accelerometer data (blue line), and the trigger data to mark the beginning of the first and last movement (black lines)-the y axis is given in m/s for the velocity data (note the accelerometer data is provided in relative units of the accelerometer) and x-axis is time, this data is for the right joint. In FIG. 9C, we show the data from the power in the movements of the right hand as a function of frequency as determined from the accelerometer data.

The following metrics are the final output for this test: total duration of test; number of movements actually performed; movement mean speed (mean and standard deviation across all movements); movement peak speed (mean and standard deviation across all movements); movement duration (mean and standard deviation across all movements); movement smoothness (mean and standard deviation across all movements); path length; tremor in the range 6-9 Hz; tremor in the range 6-11 Hz. See FIG. 9D and FIG. 9E. In other embodiments this analysis could be done in other bands, such as for example from 8 to 12 Hz or at one specific frequency. That data is then compared against the reference set or against the subject's prior set of kinematic and/or kinetic information for assessment of a movement disorder or assessment of the effectiveness of stimulation on treating the movement disorder. As above, in other embodiments, different computational methods and/or step order can be followed to determine kinematic and/or kinetic information output from the system.

In an additional example, we can further determine further information about the movement characteristics with the data recorded from the camera and the accelerometer. For example, the tremor data could be analyzed for each individual movement and correlated to the camera information to provide true directional information (i.e., the tremor as a function of movement direction or movement type) or quality of movement information. An individual system of just the accelerometer could not provide such information, because the accelerometer reports its acceleration information as a function of the internal axes of the accelerometer that are changing continuously with the patient movement. Furthermore, typical camera systems cannot provide this information because their sampling rate is generally too low (for example see similar tremor data gathered with a typical camera during the same movements), nor do they allow one to localize tremor to a specific fixed location on the body with a single fixed camera as patient movements can obscure joint locations from observation (i.e., a single camera could not provide full 3D information about the movements, and multiple cameras still cannot fill information when their views are obscured by patient movements). In certain examples, a high speed camera could be used to provide tremor data (and/or with the use of other motion analysis systems). Furthermore, in certain embodiments the combined system allows multiple levels of redundancy that allow for a more robust data set that can provide further details and resolution to the signal analysis of the patient data.

In another embodiment, resting tremor is assessed, which is assessment of tremor while the hand is at a resting position (for example evaluated from 4-6 Hz). In another embodiment, postural tremor is assessed while having a subject maintain a fixed posture with a joint. For example, a subject is asked to keep their hand still and hold it in front of their face. During the assessments for resting, action, and/or postural tremor, different frequency bands can be explored, such as frequencies or frequency bands from 0-1 Hz, 1-2 Hz, 2-3 Hz, 4-6 Hz, 8-12 Hz, and so on. The tremor frequency band could be determined based on a specific disease state, such as Essential Tremor and/or Parkinson/s Disease (or used to compare disease states).

In another embodiment, a patient's posture and/or balance characteristics are assessed. A subject is asked to stand on a force plate (e.g., a Wii Balance Board) while multiple conditions are assessed: eyes open, eyes closed, patient response to an external stimuli (e.g., an clinical evaluator provides a push or pull to slightly off balance the patient, or a mechanical system or robotic system provides a fixed perturbation force to the patient) herein referred to as sway tests (note that this set of conditions is just exemplary, and that other conditions could be completed, or just a subset of those presented. Furthermore, in certain embodiments the tests could be completed with more or less body motion sensors placed at different locations on the body, and/or the analysis can be completed for different joint(s)). During measurements with eyes open or closed, the subject is simply asked to stand on a force plate. During sway measurements, the subject is slightly pulled by a clinician (or other system, such as a mechanical or robotic system). The image capture device can optionally record and transmit these movements of the subject to the CPU, which becomes the first set of motion data. Additionally, the external body motion sensor(s) record and transmit motion data while the subject is performing this task, which becomes the second set of motion data. Data from the force plate is also acquired and transmitted to the CPU, which becomes the balance data. A trigger can be used to mark events into the recorded data. Specifically, an operator (and/or the patient) is asked to use the trigger to at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offset is Onset+15 seconds (or Onset+total length of data if recordings are shorter)). Onset, offset, and total duration are displayed to the user, who can edit them if needed. For this task, the image capture device can record and transmits joint position data (X, Y, Z) related to patient spinal, shoulder, and/or additional joint information. The accelerometer and optionally gyroscope are positioned on the subject's spinal L5 location (on the surface of the lower back) and/or other joint locations.

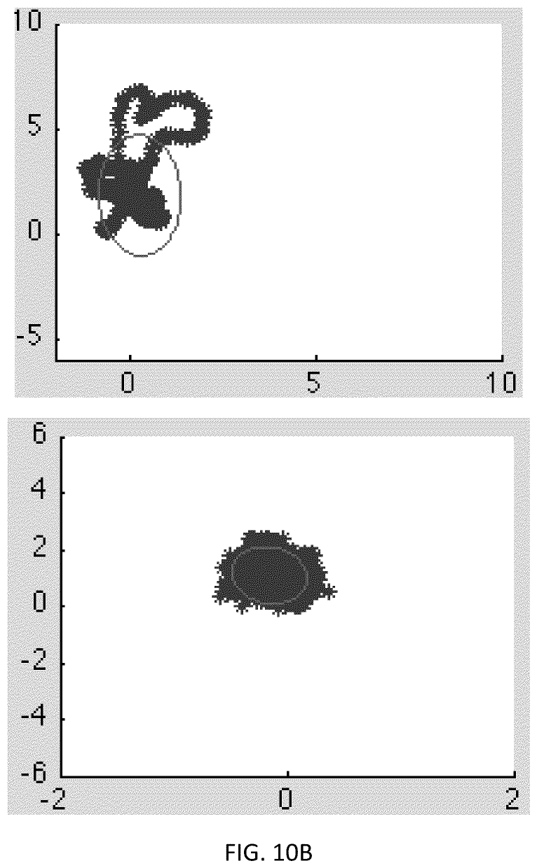

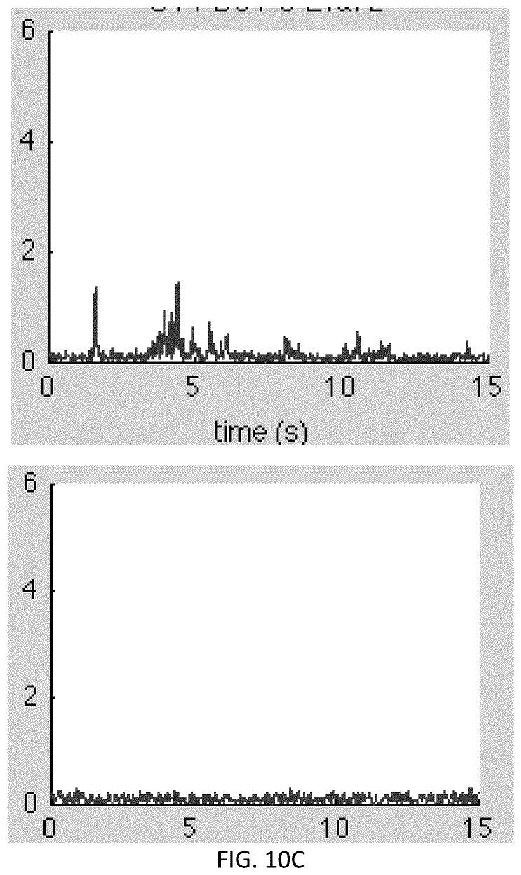

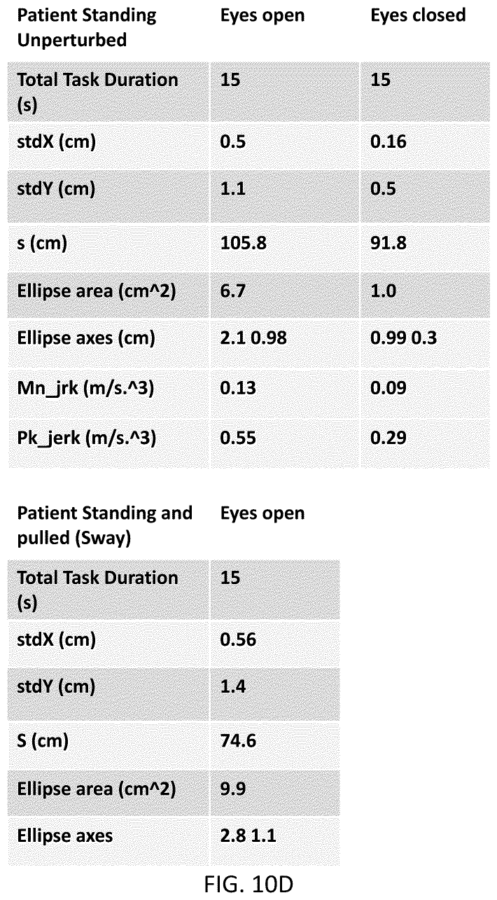

Metrics of balance are derived from the center of pressure (X and Y coordinates) recordings of the force plate. StdX and StdY are calculated as the standard deviation of the center of pressure. The path length of the center of pressure (distance traveled by the center of pressure in the X, Y plane) is also calculated. The movements of the center of pressure are fitted with an ellipse, and the area and axes of the ellipse are calculated. The axes of the ellipse are calculated from the eigenvalues of the covariance matrix; the area is the product of the axes multiplied by PI. In FIG. 10A, the weight calculated for the front and back of the left and right foot is calculated in kg, and the red line depicts a trigger mark where a clinician has determined a patient has stepped on the board and begun balancing and the second line depicts when the clinician tells the patient the test is over and they can prepare to get off the force plate, the x-axis is in until of time. In FIG. 10B, we show typical examples of data depicting patients center of gravity movements (blue), here depicted in units of length, and area ellipses depicting total movement (red)—the top part shows a patient who has been perturbed (eyes open) and swaying and the bottom part shows a patient standing without perturbation (eyes closed). The time information could be communicated on a third axis or via color coding, here for clarity it is removed in the current depiction,

Jerk is calculated by analyzing acceleration along X and Y (and Z in certain embodiments), which are calculated by differentiating accelerometer data along the relevant axes, smoothed with a moving average filter (N=5). Jerk is then calculated as root square of Jerk X and Jerk Y (and in certain embodiments as the root square of Jerk X, Jerk Y, and Jerk Z). Mean value and peak value of jerk is calculated. In FIG. 10C the jerk data, in units of position per time cubed, are provided-the top part shows a patient who has been perturbed and swaying (eyes open) and the bottom part shows a patient standing without perturbation (eyes closed)-corresponding to the data in FIG. 10B. The jerk data can be calculated in the X and Y axis from the force plate, and X, Y, and Z dimensions from the accelerometer data or image capture device data (note each captures different jerk information, for example from the force plate we could calculate jerk of the center of gravity, from the accelerometers the jerk about the individual axes of the devices, and for the camera the relative jerk data of the analyzed joints. All of these measures can be compared and registered in the same analysis space by appropriately coupling or co-registering the data as mentioned above). The image capture device can capture information about the joint positions and be analyzed similar to what is described in the above examples.

In the sway condition, all of the metrics can be evaluated as a function of the initial subject perturbation, push or pull force, derived perturbation characteristics, and/or derived force characteristics (such as rate of change, integral of force, force as function of time, etc.).

The following metrics are the final output for this test: total duration of test; StdX; StdY; path length s; ellipse area; ellipse major and minor axis; mean jerk; and peak jerk, see FIG. 10D. That data is then compared against the reference set or against the subject's prior set of kinematic and/or kinetic information for assessment of a movement disorder or assessment of the effectiveness of stimulation on treating the movement disorder. In certain embodiments, this method allows an observer to provide a controlled version of a typical Romberg test used in clinical neurology. As above, in other embodiments, different computational methods and/or step order can be followed to determine kinematic and/or kinetic information output from the system. In certain embodiments, additional results that could be assessed include: center of gravity (and/or its acceleration, velocity, position, power, and/or other derived metrics (e.g., jerk)) as a function of joint(s) analyzed; body position and/or joint angle (and/or their acceleration, velocity, position, power, and/or other derived metrics (such as average, median, and/or standard deviation of these metrics)) as a function of movement(s) analyzed; sway trajectory information (acceleration, velocity, position, power, direction, quality, and/or other derived metrics) as a function of patient perturbation force (acceleration, velocity, position, power, direction, quality, and/or other derived metrics); timing data related to the patients COG movement (e.g., time to return to center balanced point, time of sway in a certain direction(s)); and/or analysis based on individual or combined elements of these and/or the above examples.

In another embodiment for assessing gait and/or posture, a subject is asked to walk 10 meters, four different times (note that this number, joint, motion sensor(s) placement(s), and joint task is just exemplary). The image capture device optionally records and transmits these movements of the subject to the CPU, which becomes the first set of motion data. Additionally, the external body motion sensor(s) record and transmit motion data while the subject is performing this task, which becomes the second set of motion data. The trigger is used to mark events into the recorded data. Specifically, the subject is asked to use the trigger to at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offset, last value greater than 0). Onset, offset, and total duration are displayed to the user, who can edit them if needed.

For walks 1 and 2, an external body motion sensor (accelerometer and gyroscope) is positioned on the subject's left and right ankles. For this task, the image capture device can record and transmits joint position data (X, Y, Z) related to joints of the lower limbs, spine, trunk, and/or upper limbs. For this task, the image capture device can record and transmits joint position data (X, Y, Z) related to joints of the lower limbs, spine, trunk, and/or upper limbs.

For walks 3 and 4, a first external body motion sensor (accelerometer and gyroscope) is positioned on the subject's back (L5), and a second external body motion sensor (accelerometer and gyroscope) is positioned on one of the subject's ankles, preferably the right ankle. For this task, the image capture device can record and transmits joint position data (X, Y, Z) related to joints of the lower limbs, spine, trunk, and/or upper limbs.

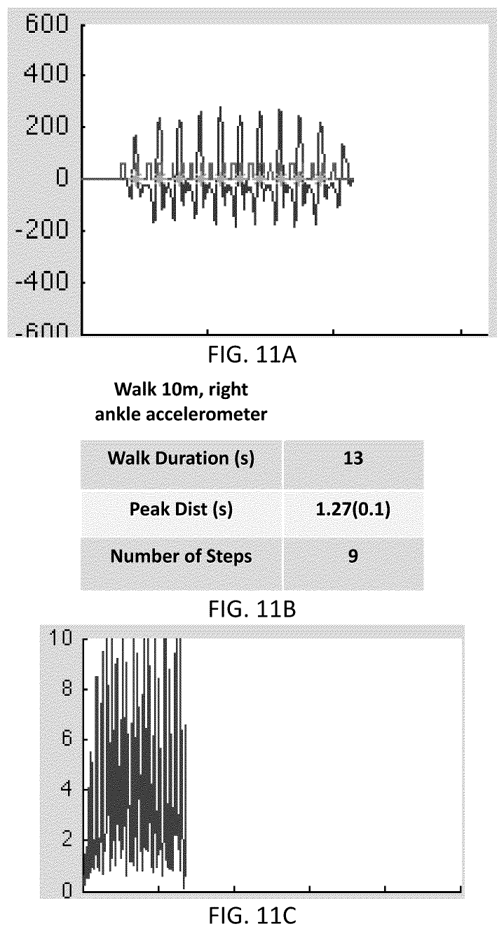

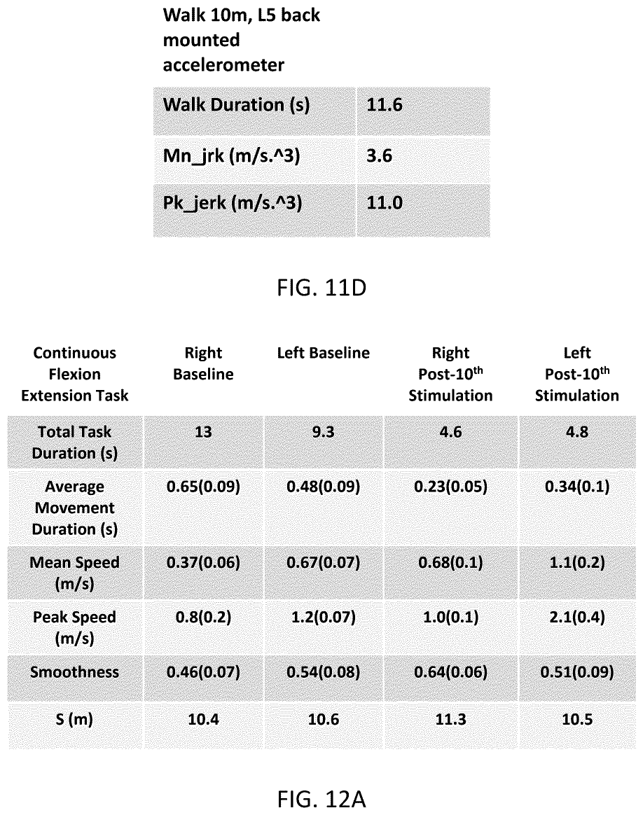

For the right ankle, acceleration metrics of gait are derived. Specifically peaks of Z rot (gyroscope data for Z) are extracted, and the distance in time between consecutive peaks is calculated (this is considered a metric of stride time). The number of strides is calculated as number of peaks +1. For example, in FIG. 11A, the peaks of the rotational component of the gyroscope along its Z axis are identified and displayed to the user (blue line in units of the gyroscopic device), the red lines show the triggering device, and the green line depicts the time instants corresponding to peaks of Z rotational component. The Y-axis is given in the relative units of the gyroscope around its Z-axis, and the X-axis in units of time. The triggering device here is activated on every step. The compiled results of this analysis are shown in FIG. 11B, demonstrating the total walk time, and longest time per right step (Peak Distance).

For the left ankle, acceleration metrics of gait are derived as described above for the right ankle, but −Zrot is used instead.

For the back, acceleration is calculated by analyzing jerk along X, Y, and Z, which is calculated by differentiating accelerometer data along X, Y, and Z. Jerk_X, Jerk_Y and Jerk_Z are then filtered with a low-pass butterworth filter (N=4, cut_off=5 Hz). Jerk is finally calculated as root square of Jerk_X, Jerk_Y, and Jerk_Z. In FIG. 11C, an example of Jerk is shown (the Y-axis is in the units of m/time\hat{ }3, X-axis in terms of time), the blue line corresponds to the period while a person is walking and the open space when the walk and task recording has stopped. Mean value and peak value of jerk is calculated. The image capture device can capture information about the joint positions and be analyzed similar to what is described in the above examples. The compiled results of this analysis are shown in FIG. 11D.

The following metrics for walks 1 and 2 are the final output for this test: total duration of test (average of test 1 and test 2); mean stride time for left ankle (average of test 1 and test 2); standard deviation of stride time for left ankle (average of test 1 and test 2); number of strides for right ankle; mean stride time for right ankle (average of test 1 and test 2); standard deviation of stride time for right ankle (average of test 1 and test 2); and number of strides for right ankle. That data is then compared against the reference set or against the subject's prior set of kinematic and/or kinetic information for assessment of a movement disorder or assessment of the effectiveness of stimulation on treating the movement disorder.

The following metrics for walks 3 and 4 are the final output for this test: total duration of test; mean jerk (average of test 3 and test 4); and peak jerk (average of test 3 and test 4). That data is then compared against the reference set or against the subject's prior set of kinematic and/or kinetic information for assessment of a movement disorder or assessment of the effectiveness of stimulation on treating the movement disorder. As above, in other embodiments, different computational methods and/or step order can be followed to determine kinematic and/or kinetic information output from the system.

Many other metrics could be determined, such as, for example, average step length, path direction (e.g., was a patient moving in a straight line or some other path), time foot is planted, stride length, range of motion of joint, relative arm and leg movement characteristics, and posture during movements.

### Wearable Components

In alternative embodiments, the system components described herein, can in part or in whole be part of a wearable item(s) that integrates some or all of the components. For example, a person could wear a suit that integrates motion analysis sensors (e.g., accelerometers) in a wearable item, with a CPU processing unit, a telecommunications component and/or a storage component to complete the analysis, diagnosis, evaluation, and/or following of a movement disorder. Or for example one could have a watch with an accelerometer connected wirelessly to a mobile phone and an external image capture device to complete the analysis, diagnosis, evaluation, and/or following of a movement disorder (in certain embodiments the image capture camera could be in a mobile phone, and/or part of a watch or wearable item). In certain embodiments the system can contain a wearable image capture device (such as for example components exemplified by a GoPro camera and/or image capture devices typically worn by the military or law enforcement). In certain embodiments, the wearable system components can be integrated (either wirelessly or via wired connections) with multiple other wearable components (such as a watch, a helmet, a brace on the lower limb, a glove, shoe, and/or a shirt). In certain embodiments, the patient could wear a shoe that has at least one sensor built into the system, such as for example a sole of a shoe that can measure the force or pressure exerted by the foot, such as for example a component that could be used to provide a pressure map of the foot, displays force vs. time graphs and pressure profiles in real time, and/or position and trajectories for Center of Force (CoF) during phases of gait.

In certain embodiments, the system can track and/or compare the results of two or more different users, for example two people could be wearing comparable wearable items, such that the items are part of the same network with at least one CPU unit, which allows the comparison of individual's wearing the devices (for example a healthy individual could be wearing a device that is simultaneously worn by another who was suffering from a movement disorder, and the individuals could perform tasks simultaneously, such that the CPU could compare the data from the wearable items to complete analysis, diagnosis, evaluation, and/or following of a movement disorder). In alternative embodiments, at least one of the wearable items can be connected or integrated with an active component(s) (such as a for example a robotic or electromechanical systems that can assist in controlled movements) so for example a healthy individual could be wearing a device that is simultaneously worn by another who was suffering from a movement disorder, and the individuals could perform tasks simultaneously, such that the CPU could compare the data from the wearable items and provide a signal that controls active components of the device worn by the individual suffering from a movement disorder to aid or assist the patient in the completion of a task (this for example could be used as part of a training or therapy protocol). In alternative embodiments, the systems could be connected via active and passive feedback mechanisms.

In yet a further alternative embodiment, multiple components and/or systems could integrate through the methods described herein and be used for the analysis of multiple individuals, such as for example following the performance of a sports team during tasks or competition or following the interaction of a patient with other individuals.

In one alternative embodiment, one could use implantable components, where at least one motion analysis component is implanted in the body of a subject being analyzed. This embodiment requires invasive procedures to place a sensor in the body.

In certain embodiments, the system can be used as a training device with or without feedback, such as in training surgeons to perform movements for surgical procedures (such as without tremor or deviation from predefined criteria), or an athlete completing balance training.

### Active System Components

In alternative embodiments, the motion analysis system may be integrated with an active component(s) (such as for example a robotic, or electromechanical system) that can assist in controlled movements), which for example could assist the patient in performing movement tasks. For example, the components could be worn by a person or placed on a person and used to assist a patient in a flexion and extension task, while the system monitors and analyzes the movement, and helps a patient complete a recovery protocol. These active components may or may not be controlled by the system, or be independent and/or have their control signals integrated with the system. Furthermore, the systems could be controlled by active or passive feedback between the different components. Furthermore, these devices can also provide data that can be used by the CPU to assess patient movement characteristics such as for example movement measurement data, trigger information, synchronization information, and/or timing information. These active components can also be used to provide stimuli to the patient during task assessment.

### Diagnosis

The system and the alternative embodiments described herein can be used diagnostically, such as to aid in or to provide the diagnosis of a disease or disorder, or to aid in or provide the differential diagnosis between different diseases or disorder states. The system can also be used as a diagnostic tool, where a diagnosis is made based on the response to a therapy as demonstrated from the motion analysis system, for example giving a suspected Parkinsonian patient a treatment of dopamine and assessing the patient's response to the drug with the motion analysis system. The system could also be used to stratify between different disease states, such as for example using the motion analysis system to determine what type of progressive supra nuclear palsy (PSP) a PSP patient has and/or to determine the severity of a disease or disorder. The system can be used to provide a diagnosis with or without the input of a clinician, and in certain embodiments the system can be used as a tool for the clinician to make a diagnosis.

In certain embodiments of the diagnostic system, the system uses a reference set of data to which a subject is compared in order to make a diagnosis or assessment of the subject. The reference set, stored on the CPU or remotely on a server operably coupled to the CPU, includes data of normal healthy individuals and/or individuals with various ailments of various ages, genders, body type (e.g., height, weight, percent body fat, etc.). Those healthy individuals and/or individuals with various ailments have been analyzed using the motion analysis system of the disclosure and their data is recorded as baseline data for the reference data set (in alternative embodiments, a reference set can be developed by modeling simulation motion data and/or a reference set could be developed from a mathematical model developed based on the analysis of assessments of healthy individuals and/or patients). The reference set of data could be based on previous measurements taken from the patient currently being assessed. A test subject is then evaluated using the motion analysis system of the disclosure and their kinematic and/or kinetic information is compared against the appropriate population in the reference set, i.e., the test subject data is matched to the data of a population within the reference set having the same or similar age, gender, and body type as that of the subject. The difference, if any, between the test subject's kinematic and/or kinetic information as compared to that of the reference data set allows for the assessment and/or diagnosis of a movement disorder in the subject. Typically, at least a 25% difference (e.g., 30%, 35%, 40%, 45%, 50%, 60%, 70%, 80%, 90%, 95%, 95%, or 99%) between the kinematic and/or kinetic information of the subject and that of the reference data set is an indication that the subject has a movement disorder. The greater the difference between the kinematic and/or kinetic information of the subject and that of the reference data set allows for the assessment of the severity or degree of progression of the movement disorder. For example, a subject with at least 50% difference (e.g., 55%, 60%, 70%, 80%, 90%, 95%, 95%, or 99%) between their kinematic and/or kinetic information and that of the reference set would be considered to have a severe form of the movement disorder. In certain diagnostic tests, just the presence of a characteristic, for example a Babinski sign, would be enough to demonstrate a patient has an ailment, which for a Babinski would demonstrate an upper motor neuron deficit in the system. In certain diagnostic evaluations, smaller differences can be demonstrated, such as for example to track a patients progress to a therapy (such as when comparing a patient's motion analysis results previous motion analysis results from a previous exam of the patient). Furthermore multiple small differences (e.g., less than 25%), for example in at least two criteria, between a tested patient and a data set can be used to make a probabilistic diagnosis that a patient suffers from a disorder (for example, in certain alternative embodiments, multiple changes, with changes as small as 1%, could be used to make a statistical model, that can have a predictive capabilities with high probability that a disease is present (such as for example with 80%, 90%, 95%, 99%,99.9% and 100% probability)-for example: a statistical model based on 10 different movement task characteristics could be assessed which makes a diagnosis based on a weighted probabilistic model, a disease diagnosis model based on derived results or grouped results (e.g., positive presence of a Babinski sign when 99 other tested criteria were not met, would still be an indication of an upper motor neuron disease), and/or model based on patient history and a result(s) derived from the motion analysis system while patients are performing a movement or set of movement tasks).

In certain embodiments, the CPU can contain and/or be connected to an external database that contains a set of disease characteristics and/or a decision tree flow chart to aid in or complete the diagnosis of a disease (additional types of databases, analyses methods, and data types which can be integrated with the method can be found in THE EVOLUTION OF BIG DATA IN NEUROSCIENCE AND NEUROLOGY incorporated hereinabove. In certain embodiments the system can take information in about the patient demographics and/or history. In certain embodiments, the CPU might direct a clinician to perform certain tests based on a patient's history and chief complaint or the clinician could have the choice to completely control the system based on their decisions. The test plan (i.e., planned tasks to be conducted and subsequent analysis) can be modified throughout the entire patient exam, based on results gathered from an ongoing exam (such as for example based on a probabilistic decision derived from the motion analysis system measured patient movement characteristics and CPU analysis) and/or clinician interaction. The system could be programed to conduct a part of an exam, such as a cranial nerve exam, or a focused exam relative to an initial presentation of symptoms or complaint of a patient, such as for example a motor exam tailored by the system to compare PSP and Parkinson's Disease, and/or other potential movement disorders.

For example, a patient might come to a clinic with complaints of slowed movement and issues with their balance including a history of falls. The patient could be fitted with a number of motion analysis sensors, such as accelerometer and gyroscopes, and be asked to perform a number of tasks while in view of an image capture system, and the data from these measurements are processed by a CPU to aid in or provide a patient diagnosis. In this example herein, the CPU might process demographic information about the patients (e.g., 72 years, male) and that the patient has a history of falls and is presenting with a chief complaint of slowed movement and complaints of balance problems. Based on this information the system could recommend a set of tasks for the patient to complete while being analyzed by the system, and/or a clinician can direct the exam (for example, based on an epidemiological data set of potential patient diagnoses from a reference set).

In this example, the doctor first instructs the patient to perform a number of tasks, such as a flexion and extension task or a combined movement task, to determine movement characteristics such as the speed, smoothness, and/or range of movement that they are moving at during the task, which is compared to a reference set of data (e.g., matched healthy individuals and/or patients suffering from various ailments). By using the motion analysis system, with the data from the body motion sensors and the image capture system, the CPU could complete the analysis exemplified as above, and compare this data to matched (e.g., age, sex, etc.) subjects who performed the same tasks. The CPU directed comparison to reference data could be made just compared to healthy individuals, to patients suffering from a pathology or pathologies, and/or both. The system analysis of the patient task performance could establish that the example patient has slowed movements (i.e., bradykinesia), indicating the potential for a hypokinetic disorder, and demonstrate that the symptoms are only present on one side of the body (note this example of patient symptoms provided herein is just exemplary and not meant to be limiting but provided to demonstrate how this diagnostic embodiment of the device could be used with an example patient).

Next, while being assessed by the motion analysis system, the patient could be asked to perform a number of additional movement tasks to assess for tremor and/or additional quality measures of movement (such as by using the system as exemplified above). This could for example establish that the patient has no evidence of postural, resting, and/or action tremor (aka kinetic tremor) relative to matched healthy subjects or patients suffering from tremor pathologies (e.g., the example patient demonstrates insignificant signs of increased power in frequency bands indicative of abnormal tremors as determined by the CPU by comparing the motion analysis system results with a reference data set).

In certain alternative embodiments the system can be designed to assess and compare tremors of different diseases such as for example Parkinsonism, multiple sclerosis, cerebellar tremor, essential tremor, orthostatic tremor, dystonic tremor, and/or enhanced physiological tremors (with each other and/or with a normal physiological tremor). As above, the tremor can be correlated with numerous conditions, such as body position, joint position and/or movement, for the diagnosis of a movement disorder.

Next while being assessed by the motion analysis system, the patient could be asked to stand still and have the posture analyzed by the system, such as by using the system as exemplified above. In this case the system analysis of the patient could for example demonstrate that the patient has a very subtle posture abnormality where they are leaning backwards while standing up relative to matched healthy subjects (indicative of rigidity of the upper back and neck muscles seen in certain pathologies in matched patients, such as those with PSP).

Next while being assessed by the motion analysis system, the patient could stand on a force plate and have their balance analyzed in number of different states (e.g., eyes open, eyes closed, feet together, feet apart, on one foot, and/or with a clinician provided perturbation (e.g., bump)), such as by using the system as exemplified above. In this case the system analysis of the patient could for example demonstrate a lack of stability (e.g., large disturbances in their center of gravity) and demonstrate a positive Romberg sign relative to healthy matched subjects and being indicative of matched patients suffering from various pathologies that negatively affect their balance (such as Parkinsonism).

Next while being assessed by the motion analysis system, the patient could then be asked to walk along a 10 meter path, turn around, walk another 10 meters back to the starting point. As described above, the patient's gait and/or posture characteristics could be analyzed and/or compared relative to matched subjects. For example, in this patient, it could be shown with the motion analysis system that the patient has a slower average gait speed and a smaller stride length than a typical matched healthy subject (furthermore it might be shown that their stride and gait characteristics were more effected on one side of the body than the other, which was comparable with their bradykinesia symptoms, and potentially indicative of Parkinsonism given the other data analyzed).

Next while being assessed by the motion analysis system, the clinician could also manually manipulate the patient's joint(s), by providing a fixed, measured, random, and/or calculated force to move a joint of the patient. This manipulation could be done while asking the patient to be passive, to resist, and/or to move in a certain manner. This manipulation could be accomplished by an external or additional integrated system, such as by a robot. The motion analysis suite could assess the joint displacement characteristics to the clinician provided manipulation. This information could be used as a measure of the patient's rigidity. There are a number of ways the motion analysis system and alternative embodiments could assess rigidity. For example, the motion analysis suite can determine the response of the joint to the clinician provided manipulation by assessing patterns of movement such as explained above (for example the magnitude of movement along a path length, directional response, power in response), or whether the trajectory or the joint displacement is continuous and smooth such as for example whether it might show a cogwheel (which presents as a jerky resistance to passive movement as muscles tense and relax) or lead-pipe (the body won't move; it's stiff, like a lead pipe) rigidity pattern. In certain embodiments, the system can be used to determine the force or characteristics of the movement perturbing the joint and the response of the joint to the manipulation, such as by using the accelerometer data of magnitude and relative acceleration direction (where in certain embodiments the exact direction in the patients' coordinate system are determined by the camera) and/or a calculation of mass of the joint (for example, the image capture device could be used to provide dimension information about the joint being moved (e.g., arm and wrist information in an elbow example), and with that information a calculation of the mass of the moved joint could be determined based on typical density information of the limb). In certain embodiments, the acceleration of the perturbation movement (i.e., the manipulation movement of the joint) could be used in lieu of force (for example, one could determine the response of a joint to an external acceleration). In certain embodiments, the force or movement characteristics that the clinician provides to manipulate or perturb the patient's joints can also be determined by having the clinician wear at least one external motion sensor (such as an accelerometer) and/or be analyzed by the motion analysis system where in certain embodiments they are also assessed by the motion capture device. Additionally, the force to manipulate a joint provided can be measured by a separate system and/or sensor and provided real-time or/at a later point to the system for analysis. In this example patient, the patient could show an absence of rigidity in the arms and legs (e.g., throughout the upper and lower limbs) as assessed by the motion analysis system.

Note, as described in the assessment and analysis of a patient's potential rigidity, in certain embodiments, the clinician could wear at least one external motion sensor and/or place at least one on or in an external instrument used as part of the exam for any part of the patient analysis. Such, as for example one could assess a patient's reflexes to an accelerating hammer, with fixed mass and shape characteristics, which has an accelerometer in it. In this example, the clinician could note normal joint reflexes in the upper and lower limb as assessed by the motion analysis system.

Next while being assessed by the motion analysis system, the patient might also be asked to hold both arms fully extended at shoulder level in front of him, with the palms upwards, and hold the position, either in a normal state, with their eyes closed, and/or while the clinician and/or system provides a tapping (such as through an active system component in certain system embodiments) to the patient's hands or arms. If the patient is unable to maintain the initial position the result is positive for pronator drift, indicative of an upper motor neuron disease and depending on the direction and quality of the movement the system could determine the cause (such as from a cerebellar cause, such as for example when forearm pronates then the person is said to have pronator drift on that side reflecting a contra-lateral pyramidal tract lesion. A lesion in the cerebellum usually produces a drift upwards, along with slow pronation of the wrist and elbow). The system could complete the analysis of the movements and comparison to a reference data set as above, and demonstrate that the example patient shows no differences in pronator drift relative to matched healthy subjects.

Next while being assessed by the motion analysis system, the patient might then be asked to remove their shoe and the clinician might place an accelerometer on the patient's big toe (if it was not used for any of the previous tasks). The physician could than manually run an object with a hard blunt edge along the lateral side of the sole of the foot so as not to cause pain, discomfort, or injury to the skin; the instrument is run from the heel along a curve to the toes (note the motion analysis system could also automate this with an active component). The accelerometer (and/or other motion sensor) and image capture device can determine whether a Babinski reflex is elicited in this patient (The plantar reflex is a reflex elicited when the sole of the foot is stimulated with a blunt instrument. The reflex can take one of two forms. In normal adults the plantar reflex causes a downward response of the hallux (flexion), which could be recorded with the system. An upward response (extension) of the hallux is known as Koch sign, Babinski response or Babinski sign, named after the neurologist Joseph Babinski. The presence of the Babinski sign can identify disease of the spinal cord and brain in adults, and also exists as a primitive reflex in infants)). The system could complete the analysis of the movements and comparison to a reference data set as above, and demonstrate that the example patient did not have a definitive Babinski.

Next while being assessed by the motion analysis system, the patient might then be given a cognitive exam (such as for example a mini mental state exam, General Practitioner Assessment of Cognition (GPCOG), Mini-Cog, Memory Impairment Screener, Language Assessment Screener, Wisconsin Card Sorting Test, Dementia Rating Scale, Hooper Visual Organization Test, Judgment of Line Orientation-Form V, Scale of Outcomes of Parkinson Disease-Cognitive, the Neuropsychiatry Inventory, and/or comparable instruments), to assess the patients' cognitive level or assess if there are any other deficits, which in certain embodiments could be conducted via components connected to and controlled by the motion analysis system CPU. The system could also gather data from the patient such as history and/or other symptom information not gathered at the onset of the exam but determined important as a result of the CPU analysis based on data gathered as part of the patient exam (for instance, whether this patient had sleep disturbances or a history of hallucinations), which could be determined from simple questions, or by connecting the motion analysis system to other systems which can assess a patient's sleep characteristics (e.g., REM sleep disturbances). For this example, as could be determined by the motion analysis system (connected to additional system components that provided a cognitive exam), this example patient could demonstrate no cognitive abnormalities that indicate severe dementia or cognitive decline compared to the CPU analyzed reference sets.

At this stage for this example, the clinician has analyzed the patient with the motion analysis system and the patient demonstrates positive signs for asymmetric bradykinesia, gait abnormalities (with issues more pronounced on one side), a slight posture abnormality indicative of rigidity in the neck and upper back but no pronounced rigidity in the peripheral joints, and poor general balance with a positive Romberg sign. Based on the results of the motion analysis system, the system and the doctor indicate that the patient has early stage Parkinson's Disease or early PSP. The doctor sends the patient home with a prescription for L-dopa and tells the patient to come back in 8 to 12 weeks (or a typical period for a patient who is responsive to the drug to begin responding to the medication).

When the patient returns, the clinician confirms that the patient has taken their course of medicine and repeats the exam with the motion analysis system as above. Unfortunately, the system reveals that patient has the same symptoms as above, did not respond to the L-dopa, but now presents with more pronounced symptoms. The motion analysis system makes a definitive diagnosis of early stage PSP and the doctor begins treating the patient with brain stimulation and tracking the patient with the motion analysis system.

In alternative embodiments, the PSP patient (or any patient being examined) could have had their eyes examined at any stage during the exam. For example on the follow-up appointment, an eye tracking system could have been used to analyze the patients vertical and horizontal gaze and specifically been used to assess whether there was a recording of restricted range of eye movement in the vertical plane, impaired saccadic or pursuit movements, abnormal saccadic or smooth pursuit eye movements, and/or other visual symptoms (the recording of other visual symptoms not explained by the presence of gaze palsy or impaired saccadic or pursuit movements, which could evolve during a PSP disease course. Symptoms include painful eyes, dry eyes, visual blurring, diplopia, blepharospasm and apraxia of eyelid opening). This eye tracking could be conducted by a connected to and/or integrated component of the motion analysis system, and the CPU analysis of this eye data, by itself and/or in combination with the other motion data could be compared to a reference set of healthy and patient performances to make the diagnosis of PSP or some other ailment.

In further alternative embodiments, the system could be connected with sensors that evaluate a patient's autonomic function such as for example urinary urgency, frequency or nocturia without hesitancy, chronic constipation, postural hypotension, sweating abnormalities and/or erectile dysfunction (which in certain embodiments could also be determined through an automated system of questions answered by the patient).

In further embodiments, the motion analysis system and connected components could be used to analyze a patients speech patterns and voice quality (such as for example through facial recognition, sound analysis, and/or vocal cord function as measured with accelerometers).

In alternative embodiments, the CPU can be programmed to analyze and track the drug history and status of the patient and be used in making diagnostic decisions or to develop more effective drug (or other therapy) dosing regimens.

In another example, another patient comes in to a clinician's office with complaints of general slowness of movement. The patient could be fitted with a number of motion analysis sensors, such as accelerometer and gyroscopes, and be asked to perform a number of tasks while in view of an image capture system, while data from these measurements are processed by a CPU to aid in or provide a patient diagnosis. The patient completes the same test as above, and demonstrates cogwheel rigidity, slowed velocity of movement, pronounced action tremor, pronounced resting tremor, pronounced postural tremor, all of which are more pronounced on the right side of the body in comparison to a healthy reference set. The system makes a diagnosis of classical Parkinson's disease.

In certain embodiments, the system would have a defined neural exam outline to conduct, based on a cranial nerve exam, a sensory exam, a motor strength exam, a sensory exam, a coordination exam, autonomic function analysis, reflexes, and/or cognitive exams (such as for example exams such as discussed in “Bates' Guide to Physical Examination and History-Taking” by Lynn Bickley MD (November 2012)).

For example, the motion analysis system could be designed to assess a patient's cranial nerves. In the first set of tasks the system is used to assess the visual acuity and eye motion of the patient. A visual monitor could be connected to the CPU, which controls visual stimuli sent to the patient, and the image capture device and/or eye tracking system could be used to record the patient movements and eye characteristics to determine the function of cranial nerves 2, 3, 4, and 6. In certain embodiments a sound recording and production device could also provide and record eye exam directions and responses (e.g., record the response from reading a line of letters, provide instructions to look upwards or to follow a light on a screen). The image capture component of the system, and potentially facial recognition software, and/or face and shoulder mounted motion sensor could be used to assess a patients ability preform facial and shoulder movements which could help in assessing the function of cranial nerve 5, 7, 9, and 11 where the patient could be instructed to complete various movements, such as example movements demonstrated to a patient on a monitor. For example, such an assessment could be used to help determine and diagnose if a patient had a stroke, where with a stoke (upper motor neuron injury) a patient might have a droopy mouth on one side and a spared forehead with the ability to raise their eyebrows (compared to another disorder such as Lyme disease where the forehead is not spared and a patient can't raise their eyebrow). In certain embodiments, the system could implement active stimuli generating components, such as for example where components could generate light touch stimuli on the location such as the forehead or cheek to assess the sensory component of the 5^{th} and 7^{th} cranial nerves, where the system could provide the stimuli to the patient and assess whether they sense the stimuli, relative to a certain location on their face as determined by the CPU and data from the image capture component (such as for example via visual feedback from the patient). The system could provide sound stimuli to assess the 8^{th} cranial nerve, based on feedback responses from the patient as to how well they hear certain stimuli. To assess the movements generated the 9^{th} and 10^{th} cranial nerve, using the system such as described above, the patient could be instructed to swallow and say “ah” and additionally assess whether their voice was horse (such as through additional sound recording and analysis methods outlined above). And finally for an evaluation of the 12 the cranial nerve the system could assess the patient as they move their tongue in various directions and through various movements (following the methods and analysis described above). As another example the motion analysis system could analyze the coordination of a patient, such as for example conducting tests such as those outlined above or other tests such as assessing things such as rapid alternating movements, flipping the heads back and forth, running and/or tapping the finger to the crease of the thumb. These tasks would be completed and analyzed as described above.

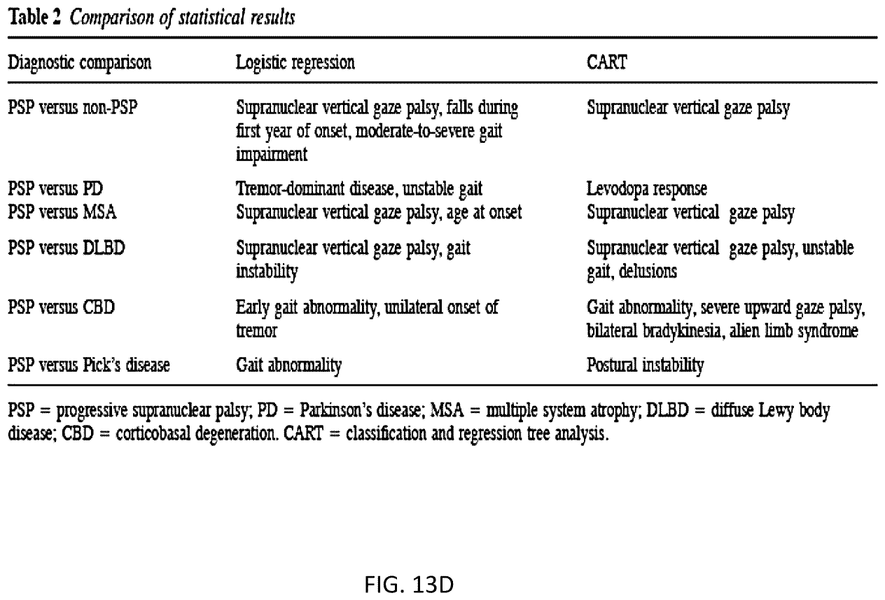

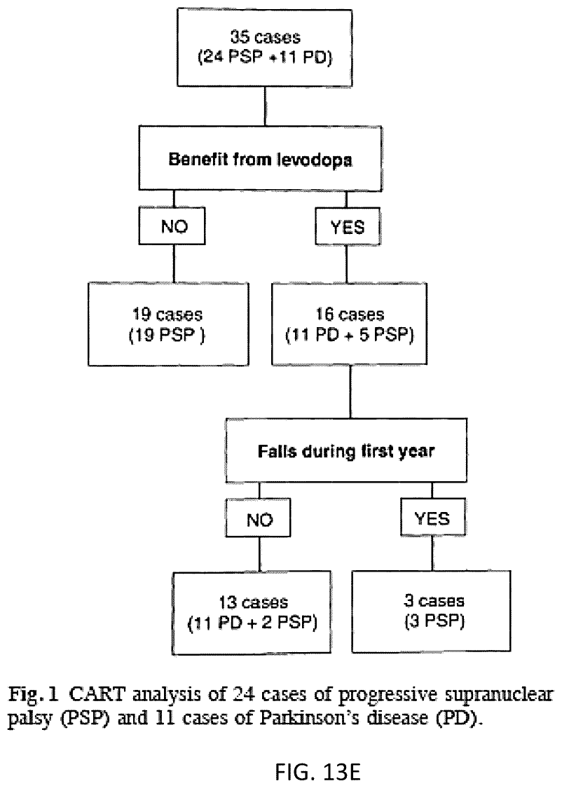

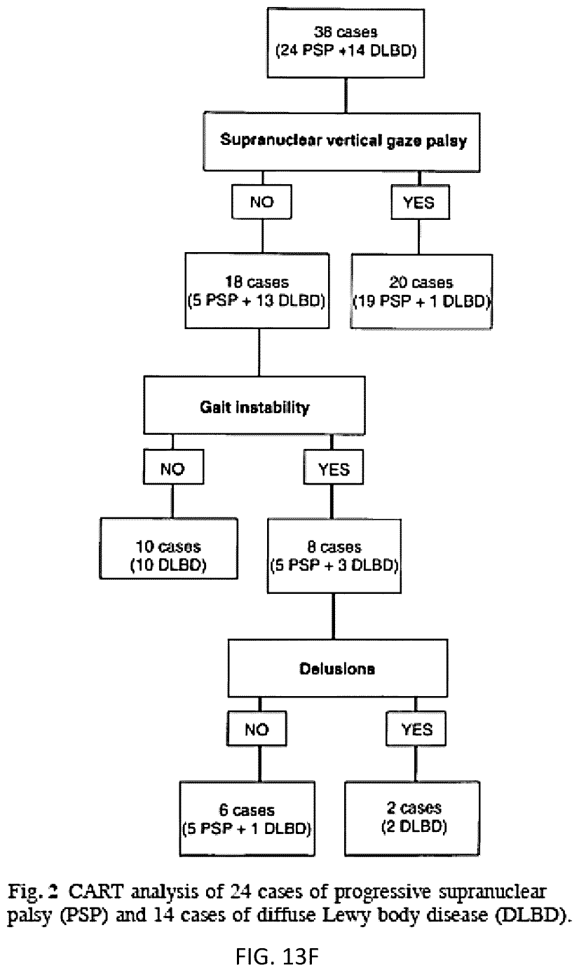

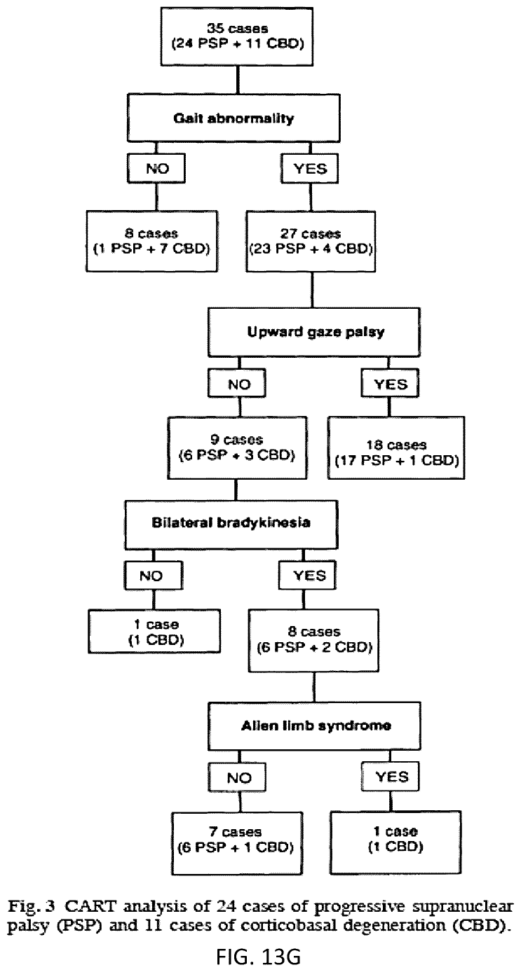

The system could have a focused neural exam based on disease characteristics that serve as part of a differential diagnosis, such as for example it could conduct a specific sub-set of a complete neural exam based on preliminary information provided by the patient. For example, a patient whose chief complaints are slowness of movement, balance abnormalities, and a history of falls could be provided a focused exam like above in the example patient diagnosed with PSP. The exam flow could be based on patient characteristics determined from across a number of previous cases, as could similarly the diagnostic criteria that the CPU uses to determine the disease state of the patient. For example, in the above PSP diagnosis example the diagnosis could be made based on defined criteria such as in FIG. 13A which is from “Liscic R M, Srulijes K, Gr€oger A, Maetzler W, Berg D. Differentiation of Progressive Supranuclear Palsy: clinical, imaging and laboratory tools. Acta Neurol Scand: 2013:127:362-370.” and/or FIG. 13B which is from “Williams et al. Characteristics of two distinct clinical phenotypes in pathologically proven progressive supranuclear palsy: Richardson's syndrome and PSP-parkinsonism. Brain (2005), 128, 1247-1258.” The motion analysis system could implement: a diagnostic flow chart based on previous studies to determine a diagnosis; a weighted decision tree based on a neuro-exam based flow chart; follow the exam and diagnostic flow of statistical studies of a disease such as could be exemplified in FIG. 13C-13G from “Litvan et al. Which clinical features differentiate progressive supranuclear palsy (Steele-Richardson-Olszewski syndrome) from related disorders? A clinicopathological study. Brain (1997), 120, 65-74.” ; a statistical prediction based on the patient criteria measured by the motion analysis system and a look up table of patient characteristics demonstrated in previous populations of patients; a probabilistic model based on past patient disease characteristics (e.g., probability of having disease given symptoms, etc.) across multiple disease states; and/or use prediction models such as those described in “The Statistical Evaluation of Medical Tests for Classification and Prediction (Oxford Statistical Science Series) by Margaret Sullivan Pepe (Dec 2004)”, “Clinical Prediction Models: A Practical Approach to Development, Validation, and Updating (Statistics for Biology and Health) by Ewout Steyerberg (October 2008)”, “Statistical Methods in Diagnostic Medicine by Xiao-Hua Zhou, Nancy A. Obuchowski, Donna K. McClish (March 2011)” the content of each of which is incorporated by reference herein in its entirety.

### Stimulation

As already mentioned above, systems and methods of the disclosure can be used with stimulation protocols. Any type of stimulation known in the art may be used with methods of the disclosure, and the stimulation may be provided in any clinically acceptable manner. For example, the stimulation may be provided invasively or noninvasively. Preferably, the stimulation is provided in a noninvasive manner. For example, electrodes may be configured to be applied to the specified tissue, tissues, or adjacent tissues. As one alternative, the electric source may be implanted inside the specified tissue, tissues, or adjacent tissues. Exemplary apparatuses for stimulating tissue are described for example in Wagner et al., (U.S. pat. publ. nos. 2008/0046053 and 2010/0070006), the content of each of which is incorporated by reference herein in its entirety.

Exemplary types of stimulation include chemical, mechanical, thermal, optical, electromagnetic, thermal, or a combination thereof. In particular embodiments, the stimulation is a mechanical field (i.e., acoustic field), such as that produced by an ultrasound device. In other embodiments, the stimulation is an electrical field. In other embodiments, the stimulation is a magnetic field. Other exemplary types of stimulation include Transcranial Direct Current Stimulation (TDCS), Transcranial Ultrasound (TUS), Transcranial Doppler Ultrasound (TDUS), Transcranial Electrical Stimulation (TES), Transcranial Alternating Current Stimulation (TACS), Cranial Electrical Stimulation (CES), Transcranial Magnetic Stimulation (TMS), temporal interference, optical stimulation, Infrared stimulation, near infrared stimulation, optogenetic stimulation, nanomaterial enabled stimulation, thermal based stimulation, chemical based stimulation, and/or combined methods. Other exemplary types include implant methods such as deep brain stimulation (DBS), microstimulation, spinal cord stimulation (SCS), and vagal nerve stimulation (VNS). Other exemplary forms of stimulation include sensory stimulation such as multi-gamma stimulation. In certain embodiments, stimulation may be provided to muscles and/or other tissues besides neural tissue. In other embodiments, the stimulation source may work in part through the alteration of the nervous tissue electromagnetic properties, where stimulation occurs from an electric source capable of generating an electric field across a region of tissue and a means for altering the permittivity and/or conductivity of tissue relative to the electric field, whereby the alteration of the tissue permittivity relative to the electric field generates a displacement current in the tissue. The means for altering the permittivity may include a chemical source, optical source, mechanical source, thermal source, or electromagnetic source.

In other embodiments, the stimulation is provided by a combination of an electric field and a mechanical field. The electric field may be pulsed, time varying, pulsed a plurality of time with each pulse being for a different length of time, or time invariant. Generally, the electric source is current that has a frequency from about DC to approximately 100,000 Hz. The mechanical field may be pulsed, time varying, or pulsed a plurality of time with each pulse being for a different length of time. In certain embodiments, the electric field is a DC electric field.

In other embodiments, the stimulation is a combination of Transcranial Ultrasound (TUS) and Transcranial Direct Current Stimulation (TDCS). Such a combination allows for focality (ability to place stimulation at fixed locations); depth (ability to selectively reach deep regions of the brain); persistence (ability to maintain stimulation effect after treatment ends); and potentiation (ability to stimulate with lower levels of energy than required by TDCS alone to achieve a clinical effect).

In certain embodiments, methods of the disclosure focus stimulation on particular structures in the brain that are associated with arthritic pain, such as the somatosensory cortex, the cingulated cortex, the thalamus, and the amygdala. Other structures that may be the focus of stimulation include the basal ganglia, the nucleus accumbens, the gastric nuclei, the brainstem, the inferior colliculus, the superior colliculus, the periaqueductal gray, the primary motor cortex, the supplementary motor cortex, the occipital lobe, Brodmann areas 1-48, the primary sensory cortex, the primary visual cortex, the primary auditory cortex, the hippocampus, the cochlea, the cranial nerves, the cerebellum, the frontal lobe, the occipital lobe, the temporal lobe, the parietal lobe, the sub-cortical structures, and the spinal cord. Stimulation and the effects of stimulation on a subject can be tuned using the data obtained from this system. Tuning stimulation and its effects are discussed, for example in U.S. pat. publ. no. 2015/0025421, the content of which is incorporated by reference herein in its entirety. Furthermore, the motion analysis system can be used as part of a DBS stimulation parameter tuning process.

In certain embodiments, stimulation and the motion analysis system can be coupled to aid in the diagnosis of a disorder. For example, brain stimulation can be applied to a specific brain area that is expected to be affected by a disease being tested for. The response of joints that are connected to the brain area can be assessed by the motion analysis system. And the motion analysis system analysis of these movements in conjunction with the stimulation response can be used to aid in the diagnosis of a disease (for example, if a patient as being tested for a lesion to the right primary motor cortex hand area of the patient under study, stimulation to the left primary cortex is expected to generate a diminished response of hand motion in the presence of a lesion).

A combined stimulation and motion analysis system could also be used to determine mechanisms of a disease or disorder, and/or methods for more appropriately treating the disease or disorder. For example, we found that stimulation to a Parkinson's Disease patient's primary motor cortex had a benefit on certain symptoms of the disease as demonstrated by the motion analysis system, and in turn we could look at those responses to stimulation to compare their differential response to determine additional therapies and explore fundamental mechanisms of the disease (such as for example comparing the differential effect of stimulation on a patient's balance with their eyes open and closed, and using this and other data to determine the impact of the disease on the patient's direct and indirect pathway, and then in turn adapting the location of stimulation based on the motion analysis data results and knowledge of these pathways to target a more effective area of the brain).

### Additional Exemplary Movements and Assessments for Use With the Motion Analysis System

The systems described herein can analyze any movement or static position of a joint, limb, and/or body part as part of the processes described herein such as for example discrete movements, continuous movements, compound movements, holding a static position, moving between static positions, extension, flexion, rotation, abduction, adduction, protrusion, retrusion, elevations, depression, lateral rotation, medial rotation, pronation, supination, circumduction, deviation, opposition, reposition, inversion, eversion, dorsiflexion, plantarflexion, excursion, medial excursion, lateral excursion, superior rotation, inferior rotation, hyperflexion, retraction, reposition, hyperextension, lateral movements, medial movements, movement of a body part relative to another body part, flexing a muscle, discrete flexion of a limb, discrete extension of a limb, continuous flexion of a limb, continuous extension of a limb, rotation of a limb, opening of a hand, closing of a hand, walking, standing, and/or any combination thereof. This can be used for any limb or joint (e.g., pivot, hinge, condyloid, saddle, plane, ball, and socket) and/or any combination thereof. The systems described herein can analyze any movement or static position of a joint, limb, and/or body part as part of the processes described herein such as for example for or with a physical exam, orthopedic exam, neurological exam, quantitative sensory testing, reflex assessment, assessing the integrity of a joint, physical therapy, stroke assessment, drug use diary, Parkinson's Disease assessment, balance testing, step tests, Romberg tests, functional reach tests, single leg balance testing, range of motion tests, and/or movement disorder assessment. The systems described herein can analyze any movement or static position of a joint, limb, and/or body part as part of the processes described herein such as for example used with or for determining the UPDRS scale, PROMIS scales, Apathy: Apathy Scale (AS), Apathy: Lille Apathy Rating Scale (LARS), Autonomic Symptom: Composite Autonomic Symptom Scale, Blepharospasm: Blepharospasm Disability Index (BSDI), Depression: Beck Depression Inventory (BDI), Depression: Cornell Scale for Depression in Dementia (CSDD), Depression: Geriatric Depression Scale (GDS), Depression: Hamilton Rating Scale for Depression (HAM-D), Depression: Hospital Anxiety and Depression Scale (HADS), Depression: Montgomery-Asberg Depression Rating Scale (MADRS), Depression: Zung Self-Rating Depression Scale (SDS), Dyskinesia: Rush Dyskinesia Rating Scale, Dyskinesia: Abnormal Involuntary Movements Scale (AIMS), Dyskinesia: Unified Dyskinesia Scale (UDysRS), Dyskinesia: Parkinson's Disease Dyskinesia Scale (PDYS-26), Dystonia: Cervical Dystonia Impact Scale (CDIP-58), Dystonia: Craniocervical Dystonia Questionnaire (CDQ-24), Dystonia: Fahn-Marsden Dystonia Rating Scale (FMDRS), Dystonia: Toronto Western Spasmodic Torticollis Rating Scale (TWSTRS), Fatigue: Fatigue Severity Scale, Fatigue: Parkinson Fatigue Scale, Fatigue: Multidimensional Fatigue Inventory, Parkinson's disease: Core assessment program for surgical interventional therapies in Parkinson's disease (CAPSIT-PD), Parkinson's disease: Hauser Diary, Parkinson's disease: Hoehn and Yahr Scale, Parkinson's disease: Non-Motor Symptoms Questionnaire (NMSQ), Parkinson's disease: Non-Motor Symptoms Scale (NMSS), Parkinson's disease: Scales for Outcomes in Parkinson's disease, Parkinson's disease: Movement Disorder Society-Unified Parkinson's Disease Rating Scale (MDS-UPDRS), Parkinson's disease: Unified Parkinson's Disease Rating Scale (UPDRS), Parkinson's disease: Wearing Off Questionnaire-9, Parkinson's disease: Wearing Off-19, Wearing Off-Quick Questionnaire, Psychosis: Brief Psychiatric Rating Scale (BPRS), Psychosis: Neuropsychiatric Inventory (NPI), Psychosis: Positive and Negative Syndrome Scale for Schizophrenia (PANSS), Psychosis: Scale for the Assessment of Positive Symptoms (SAPS), Quality of Life: European Quality of Life Scale (EQ-5D), Quality of Life: Short Form Health Survey (SF-36), Quality of Life: Parkinson's Disease Questionnaire (PDQ-39), Quality of Life: Parkinson's Disease Questionnaire (PDQ-8) Short Form, Quality of Life: Parkinson's Disease Quality of Life Questionnaire (PDQL), Quality of Life: Scales for Outcomes in Parkinson's Disease-Psychosocial, Quality of Life: Parkinson's Impact Scale (PIMS), Quality of Life: Nottingham Health Profile, Quality of Life: Sickness Impact Profile (SIP), Quality of Life: Parkinson's Disease Quality of Life Scale (PDQUALIF), Sleep: Parkinson's Disease Sleep Scale (PDSS), Sleep: Pittsburgh Sleep Quality index (PSQI), Sleep: SCOPA Sleep Scale (SCOPA), Voice: Vocal Performance Questionnaire (VPQ), Voice Handicap Index, MDS-Unified Parkinson's Disease Rating Scale (MDS-UPDRS), Rush Video-Based Tic Rating Scale, MDS Non-Motor Rating Scale (MDS-NMS), Scales for Outcomes in Parkinson's Disease Autonomic Dysfunction (SCOPA-AUT), Cortical Basal ganglia Functional Scale (CBFS), Scales for Outcomes in Parkinson's Disease-Diary Card (SCOPA-DC), Gastrointestinal Dysfunction Scale for Parkinson's Disease (GIDS-PD), Scales for Outcomes in Parkinson's Disease Psychiatric Complications (SCOPA-PC), Global Assessment Scale for Wilson's Disease (GAS for WD), Scales for Outcomes in Parkinson's Disease-Psychosocial Functioning (SCOPA-PS), Global Dystonia Severity Rating Scale (GDS), Scales for Outcomes in Parkinson's Disease Sleep (SCOPA-Sleep; SCOPA-S), Modified Bradykinesia Rating Scale (MBRS), Scales for Outcomes in Parkinson's Disease-COGnition (SCOPA-COG), Non-Motor Symptoms Questionnaire (NMSQ), Short Parkinson's Evaluation Scale (SPES)/Scales for Outcomes in Parkinson's Disease-Motor Function (SPES/SCOPA-Motor), Non-Motor Symptoms Scale for Parkinson's Disease (NMSS), The Non-Motor Fluctuation Assessment (NoMoFA) Questionnaire, PKAN Disease Rating Scale (PKAN-DRS), UFMG Sydenham's Chorea Rating Scale (USCRS), Progressive Supranuclear Palsy Clinical Deficits Scale (PSP-CDS), Unified Dyskinesia Rating Scale (UDysRS), Quality of Life in Essential Tremor Questionnaire (QUEST), Unified Dystonia Rating Scale (UDRS), Montreal Cognitive Assessment (MoCA), Mini Mental State Exam, Rating Scale for Psychogenic Movement Disorders (PMD), Unified Multiple System Atrophy Rating Scale (UMSARS), Numerical Rating Scale (NRS), Visual Analog Scale (VAS), Defense and Veterans Pain Rating Scale (DVPRS), Adult Non-Verbal Pain Scale (NVPS), Pain Assessment in Advanced Dementia Scale (PAINAD), Behavioral Pain Scale (BPS), Critical-Care Observation Tool (CPOT), Neonatal Pain, Agitation, and Sedation Scale (N-PASS), Neonatal/Infant Pain Scale (NIPS), Neonatal Facial Coding System (NFCS), CRIES, Faces Legs Activity Cry and Consolability (FLACC), Revised-FLACC, Non Communicating Children's Pain Checklist (NCCPC-R), Children's Hospital of Eastern Ontario Pain Scale (CHEOPS), Wong-Baker Faces scale, Numerical Rating Scale (NRS), Visual Analog Scale (VAS), pain diaries, drug use diaries, pain history, drug use history, symptom severity, symptom duration, symptom chronicity, pain severity, pain duration, pain chronicity, neuropathic pain symptom inventory (NPSI), American pain foundation diary, west haven yale multidimensional pain inventory, The International Classification of Headache Disorders, Pain Drawing, Pain Numeric Rating Scale (NRS), Defense and Veterans Pain Rating Scale (DVPRS), Pain Outcomes Questionnaire (POQ), Pain Assessment in Non-Communicative Adult Palliative Care Patients, Boston Carpal Tunnel Questionnaire, Western Ontario and McMaster Universities Arthritis Index (WOMAC), Michigan hand outcomes questionnaire, Multidimensional Pain Inventory, Physical therapy scales, stroke scales, Alder Hey Triage pain Scale, Behavioral Pain Scale, Brief Pain Inventory, Checklist for Nonverbal Pain Indicators, Clinical Global impression, Critical Care Pain Observation Tool, Scales for joint assessments (e.g., Boston Carpal Tunnel Questionnaire), Neuropathic Pain Symptom Inventory, Quantitative Sensory Testing, COMFORT Scale, Dallas Pain Questionnaire, Discomfort in Dementia Scale, Descriptor Differential Scale(DDS), Edmonton Symptom Assessment System, Lequesne-Algofunctional Index, Mankowski Pain Scale, McGill Pain Questionnaire, Neck pain and Disability Scale, OSWESTRY Pain Disability Index, Pediatric Pain Questionnaire, Support Team Assessment, Verbal Rating Scale, all scales and/or assessments found in the NIH Common Data Elements Repository (https://cde.nlm.nih.gov/), and/or any combination thereof. As for these lists, and others within this document, they should be considered exemplary and not limiting.

### Prediction, Inference, and/or Optimization

Aspects of the disclosure makes use of a motion analysis system diagnostic tool (motion analysis system as described for example in PCT/US14/64814) for quantitative and objective assessment of motor symptoms in Parkinson's Disease (PD), stroke, or any such pathology that affect human movement. The motion analysis suite employs a toolbox of computational methods (e.g., statistical algorithms, machine learning algorithms, optimization methods) such as for example to assess patient movement kinematics and kinetics; reduce data dimensionality; classify patient disease characteristics; highlight patient symptomology; identify patient risk characteristics; predict disease progression; predict motor behavior; predict the response to treatment; and/or tailor a patients treatment course. For example, the motion analysis suite could employ statistical and machine learning algorithms, based on the patient motion data that is recorded during an exam to improve patient diagnoses and evaluation. For example, early diagnosis of PD is quite challenging, and approximately 20% of new patients go mis-or un-diagnosed; and the motion analysis suite can be used in the differential diagnosis of PD and assist the care giver in making a proper disease diagnosis. Furthermore, disease progression is tracked using coarse clinical scales, such as the Unified Parkinson's Disease Rating Scale (UPDRS), which suffer from limited resolution and high intra-and inter-rater variability; the motion analysis suite could address these limitations, where the motion analysis system is used to measure PD motor symptoms, quantify disease severity, and facilitate diagnosis (such as through statistical algorithms and/or machine learning algorithms). The system may include a battery of portable and/or wearable sensors (including a 3D motion capture video camera (classic RGB and infrared depth-based imaging), inertial sensors, force sensors, and/or a force plate), which can be used for monitoring and quantifying subjects' motor performance during assessments, such as a UPDRS III focused motor exam. Quantitative metrics can be derived from the motion analysis suite recordings to measure primary motor symptoms (e.g., bradykinesia, rigidity, tremor, postural instability). The data from the motion analysis system can be used to build statistical models to extract a low dimensional representation of disease state and to predict disease severity (e.g., UPDRS3). Kinematic/kinetic data not classically captured with clinical scales, such as the UPDRS3 can be identified, including joint kinematics of position, movement trajectory, and movement quality across the motor system, to build full body models of disease state. The computational models can predict response to therapy based on motion analysis suite data by comparing motion analysis suite measures of patients in different states of therapy (such as in their ‘On’ and “Off’ states (i.e., on or off levodopa) or in different states of Deep Brain Stimulation (e.g., different stimulation pulse frequencies) for Parkinson's patients), or based on a database of past treated patients and their response to various therapies (e.g., Deep Brain Stimulation (DBS) for Parkinson's patients). The entire computational package of the motion analysis suite, including the kinematic/kinetic analysis software, can be combined in a patient-tracking database, capable of providing motion analysis system data that enhances classical clinical scale information (e.g., UPDRS information).

The motion analysis suite could employ statistical and machine learning algorithms, based on the patient motion data that is recorded during an exam to improve patient prognosis. For example, prediction of recovery from stroke can be quite challenging; and the motion analysis suite can be employed to predict the likelihood of the patient recovering from stroke in the acute setting or in chronic state. By integrating the different proposed individual sensor types, the system can for example first assist in perform more accurate, less variable motor exams, and symptom assessments with higher resolutions than classic clinical scales as the sensors can be used to objectively track and measure patient movements without subjective limitations of typical clinical assessments (Taylor-Rowan, M. et al, 2018, https://www.ncbi.nlm.nih.gov/pubmed/29632511). Furthermore, stroke is a multi-symptom disease of varied, yet often correlated symptoms, which is necessarily described in a “probabilistic” manner, especially when predicting motor recovery (Stinear et. al., 2007, https://www.ncbi.nlm.nih.gov/pubmed/17148468). Machine learning algorithms can be implemented to generate predictions of clinical scales (such as the Fugal Meyer Stroke scale, or the NIH Stroke Scale); Predictions of motor recovery based on integrated symptom assessment; and/or Patient classification based on statistical algorithms (e.g., sensor-based movement kinematics data can be collected during assessments with the motion analysis suite along and combined with data from past exams and/or data derived from typical patient characteristics to build a generalized linear model which predicts a patients stroke scale scores or likelihood of recovery based on the motion analysis data input (and or other clinical information collected from the patient)). As the motion analysis suite could make use of data collected from a single joint or across multiple joints throughout the body, the system allows for the development of both single joint and full body models of disease impact on movement. The computational approach with the motion analysis suite can build upon the integration of sensors that provides for a synchronized data acquisition of patient kinematics and the statistical algorithms can be employed to computationally analyze the stroke injury state, through data dimensionality reduction and prediction methods to provide the clinician with a tool to aid and augment the classic evaluation process.

In certain embodiments a motion analysis suite can aid care providers in motor symptom assessments, differential diagnosis, disease diagnosis, patient/disease classification, prediction of patient recovery, prediction or assessment of disease progression, prediction or assessment of treatment outcome, guiding treatment decisions (e.g., type, course (e.g., dose, duration, delivery timing)), treatment tuning or optimization, prognosis, prediction of new symptoms development, prediction of hospitalization risk, prediction of needed level of assistance, diet optimization, and/or dosing of a treatment. The system can include software to derive quantitative movement kinematic/kinetic-based motor evaluations; computational, statistical and/or machine learning algorithms for data reduction, data modeling, predictions of clinical scales and/or prognostic potential for disease recovery, predictions of clinical scales, prediction of response to therapy, guidance of therapy to a particular response, and/or tuning of therapy to particular response. As above, the computational system(s) can be integrated with a database of patient clinical, patient demographic data, and/or disease specific data which can be used as part of the statistical and/or machine learning calculations and assessments (additional types of databases, analyses methods, and data types which can be integrated with the method can be found in THE EVOLUTION OF BIG DATA IN NEUROSCIENCE AND NEUROLOGY incorporated hereinabove. The system itself can be designed such that the statistical and/or machine learning calculations and assessments can continually improve their capability (e.g., accuracy of predictions, resolution of assessments) based on the access to database(s) of patient clinical and/or demographic data and/or past calculation or assessment results.

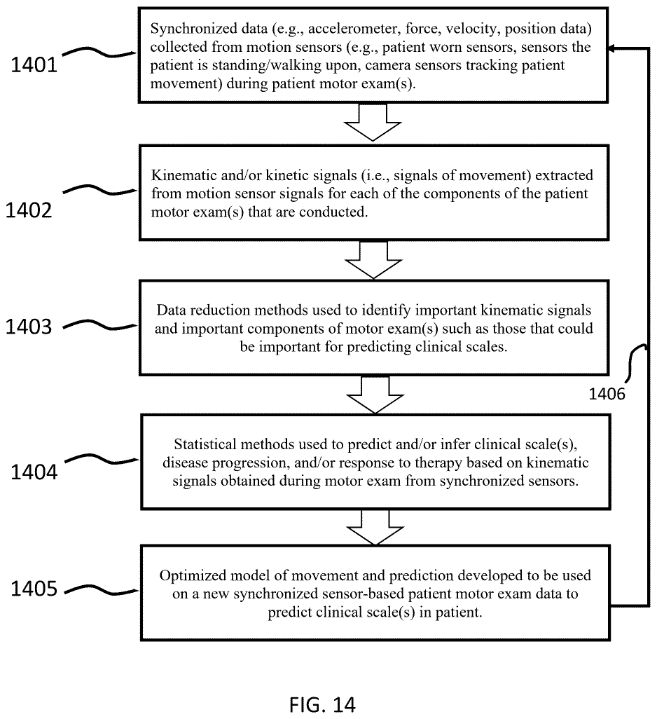

For example, in an embodiment of a motion analysis suite of the disclosure with an included analysis, data reduction, and prediction suite one could predict the UPDRS of a patient that underwent an exam and optimize the motion analysis suite for future predictions. The step-by-step methods of such an example are described schematically in FIG. 14. It will be understood that of the methods described in FIG. 14, as well as any portion of the systems and methods disclosed herein, can be implemented by computer, including the devices described above, and the process for prediction and process optimization can be implemented with such a system as exemplified. The method is only exemplary and not limiting, and the skilled artisan will appreciate that other computational steps and/or algorithms employed which are not mentioned here may be used with systems of the disclosure and that the computational steps and/or algorithms employed chosen will be chosen to allow for assessment, diagnosis, prognosis, classification, and/or therapy tuning of the movement disorder being studied. In this example, first a patient will undergo a motion analysis system focused motor exam (such as those exemplified above), as depicted in 1401 with a motion analysis system, such as depicted in FIG. 1 to collect synchronized data of patient movements. The motion analysis system could include at least one image capture device, motion sensor (e.g., accelerometer, gyroscope), force plate, and/or alternate sensor as described above (such as for example 1 image capture device; or 2 image capture devices; or 1 image capture device, 1 combined accelerometer/gyroscope sensor, and 1 force plate; or 1 image capture device, 1 combined accelerometer/gyroscope sensor, 1 force plate, and 1 sound recording device; or any such permutation of sensors). If just one recording sensor/device is used to gather data from a patient, it should be understood it is not synchronized across other sensors, but the time signal associated with the data is recorded as exemplified in the earlier descriptions. A computational system (e.g., computer(s), tablet(s), phone(s)), either connected from directly to the sensors (through a wired connection) or through a wireless connection, or that has access to the synchronized data from the motor exam (such as through storage media) can then implement methods such as those exemplified above to extract kinematic and/or kinetic signals from the data (i.e., any signal of movement), as depicted in 1402. The kinematic and/or kinetic signals (i.e., signal of movement) can then be reduced in dimension, 1403, so one could identify important kinematic and/or kinetic signals determined from the synchronized data recording from battery of recording sensor/device(s) used in the motor exam. Data reduction methods that can be employed in step 1403 include for example Dimensionality Reduction (e.g., Principal Component Analysis (PCA), Independent Component Analysis, Wavelet transforms, Attribute Subset Selection, Factor Analysis), Numerosity Reduction (e.g., Parametric (e.g., Regression and Log-Linear method), Non-Parametric (e.g., histogram, clustering, sampling (Simple random sample without replacement (SRSWOR) of size s, Simple random sample with replacement (SRSWR) of size s, Cluster sample, Stratified sample), Data Cube Aggregation), feature selection methods (e.g., missing value ratio, low variance filter, high correlation filter) and Data compression methods). Additional examples of methods of data reduction can be employed in step 1403 are exemplified in (Statistical Analysis of Complex Data: Dimensionality reduction and classification methods, M. Fordellone, 2019, LAP LAMBERT Academic Publishing; The Data Science Handbook, F. Cady, 2017, John Wiley & Sons, Inc. ; A Primer in Data Reduction: An Introductory Statistics Textbook, A. S. C. Ehrenberg, 2007, Wiley) the content of each of which is incorporated by reference herein in their entirety. This list of data reduction methods is only exemplary and not limiting, and the skilled artisan will appreciate that other data reduction methods not mentioned here may be used with systems of the disclosure and that the data reduction methods chosen will be chosen to allow for appropriate kinetic signal identification. For example, when implementing a PCA methodology, one could identify a kinematic signal from a particular sensor which contributes most to the variability of the principal component of a model built from the motor exam and a different kinematic signal from a particular sensor which contributes the least to the variability of the principal component of a model built from the motor exam. One could then implement a new model of patient movement signals based on the kinematic signal from a particular sensor which contributes most to the principal component of a model built from the motor exam and remove the different kinematic signal from a particular sensor which contributes the least to the principal component of a model built from the motor exam. In addition to using the data reduction methods on the kinetic and/or kinematic signals (i.e., signals of movement), one could also use data reduction methods on demographic and clinical information determined from or about the patient, which can be determined for example from a patient's medical records, separate patient evaluations (e.g., demographic information, past clinical exam results (e.g., lab work, imaging, electrophysiology), and/or database information (e.g., from comparable patients undergoing comparable exams, from typical patients from a pathology being assessed, from modeling work of pathology under assessment). For example, if one was conducting a UPDRS3 motor exam evaluation in a patient, and the patient was examined by a physician (who assigned a UPDRS3 scale to the patient) and the patient was assessed with a motion analysis system (with at least for example an image capture device, a accelerometer/gyroscope unit, or a force plate) one could conduct data reduction methods, such as a PCA, on the combined model determined from both the clinician based UPDRS3 scores (such as from the individual questions that make up the part 3 UPDRS exam) and from the kinematic and/or kinetic signals determined that were derived from the synchronized data that was gathered during the motion analysis system recordings during the motor exam, and individual PCA models for each the clinician based UPDRS3 scores (such as from the individual questions that make up the part 3 UPDRS exam) and from the kinematic and/or kinetic signals determined that were derived from the synchronized data that was gathered during the motion analysis system recordings during the motor exam. The comparison of the models would allow one to determine elements of each model that contribute the principal component and reduce the model dimensions. This dimension reduction data can be used as an input to the next step, and/or used to provide the operator with information of data dimensionality useful in the motor exam and/r patient assessment with the motion sensors and/or clinical scales assessed. Next, 1404, one could employ statistical methods to predict and/or infer a patient's clinical scales (e.g., UPDRS1, UPDRS2, UPDRS3, UPDRS4, UPDRS, Fugl Meyer, Fugl Meyer upper limb, Fugl Meyer lower limb, NIH Stroke Scale (NIHSS), Barthel Index, modified NIHSS, Motor Activity Log (MAL), Wolf Motor Function Test, Action Research Arm Test (ARAT), Motor Assessment Scale, Nine Hole Peg Test, Jebsen Taylor Hand Test, the Box and Block test, Chedoke-McMaster Stroke Assessment Scale, Chedoke Arm and Hand Activity Inventory, the Ashworth scale, the modified Ashworth scale, Rankin scale, modified Rankin scale, The Short Form 36 (SF-36), Stroke Specific Quality of Life scale (SS-QOL), Euro-QOL, the Postural Assessment Scale for Stroke (PASS), the Berg Balance Scale, Stroke Rehabilitation Assessment of Movement (STREAM), Clinical Outcome Variables (COVS), Functional Ambulation Categories (FAC), Functional Independence Measure (FIM), CIHI-National Rehabilitation Reporting System, Frenchay Activities Index (FAI), Modified Rankin Handicap Scale (MRS), Rivermead Mobility Index (RMI), Rivermead Motor Assessment (RMA), Six-Minute Walk Test (6MWT), Timed “Up & Go” Test (TUG), Romberg test, Rise from Chair Test, Timed Step Test, Canadian Occupational Performance Measure, LIFE-H (Assessment of Life Habits, London Handicap Scale (LHS), Nottingham Health Profile (NHP), and/or scales found in the Evidence-Based Review of Stroke Rehabilitation “Outcome Measures in Stroke Rehabilitation” by K. Salter, et. al., http://www.ebrsr.com/sites/default/files/Chapter%2020_Outcome%20Measures.pdf ) or other patient outcome information (e.g., disease progression and/or past, current, and/or future response to a treatment, such as for example from pharmaceutical treatment, stimulation treatment (e.g., Electrosonic Stimulation, DBS, TMS), and/or physical therapy treatment) based on the kinematic signals extracted from the synchronized data during the motor exams. In addition to (or in replacement of) the kinematic and/or kinetic signals extracted from motion sensor signals for components of the motor exam, one could use data on demographic and clinical information determined from or about the patient, which can be determined for example from a patient's medical records, separate patient evaluations (e.g., demographic information, past clinical exam results (e.g., lab work, imaging, electrophysiology), and/or database information (e.g., from comparable patients undergoing comparable exams, from typical patients from a pathology being assessed, from modeling work of pathology under assessment) to predict or infer a patient's clinical scales. For example, one could develop a linear model for predicting the UPDRS3 based on patient signals of movement extracted from the motion sensor signals. This can be done for a single patient, or multiple patients, across a single observation or multiple observation (e.g., patient observation could occur on separate days, such as those separated over a period of time to develop models of disease progression). In this example, one could develop a model that predicts UPDRS3 based on a linear model developed from signals of movement extracted from motion sensor signals from multiple observations across multiple patients at multiple time points, and subsequently use this linear model to predict the past, current, or future UPDRS3 scores in a new patient based on signals of movement extracted from motion sensor signals during a motor exam. Numerous statistical methods of prediction and/or inference can be employed such as regression modeling, generalized linear modeling, generalized nonlinear modeling, least absolute shrinkage and selection operator (LASSO), LASSO or elastic net regularization for linear models, linear support vector machine models, Empirical risk minimization (ERM), neural network learning, such as those are exemplified in (Applied Predictive Modeling, M. Kuhn, K. Johnson (Author), 2018, Springer; Handbook of Deep Learning in Biomedical Engineering), V. E. Balas, B. K. Mishra, R. Kumar, 2021, Academic Press; Statistical and Machine Learning Data Mining, B. Ratner, 2011, CRC Press) the content of each of which is incorporated by reference herein in their entirety. The model(s) of prediction and/or inference can further be optimized via additional machine learning/artificial intelligence (AI) methods such as deep learning. Methods used herein could, for example, be selected from the examples listed at page 21. The list of statistical methods (including machine learning, AI, and optimization methods) for prediction and inference, is only exemplary and not limiting, and the skilled artisan will appreciate that other prediction and inference methods not mentioned here may be used with systems of the disclosure and that the statistical methods chosen will be chosen to allow for appropriate methods to prediction or inference of a patient's clinical scales or other patient outcome information (e.g., disease progression or past, current, and/or future response to a treatment,) based on the kinematic signals extracted from the synchronized data during the motor exams. The model(s) of prediction or inference could use multiple steps, and/or multiple methods (such as for example using one method to optimize the parameters of a second method for optimal predictive value such as using a LASSO model to guide a generalized linear model), and/or as inputs to other models (or to themselves when using recursive methods). An optimized model of movement can be developed, 1405, identifying the signals (e.g., signals of movement, signals from the clinical history of the patient, signals determined from clinical examinations of the patients, signals of disease characteristics determined from disease databases) with the most predictive value to both determine an improved motor exam (e.g., potentially with less movement requirements such that a patient's burden would be reduced, or with additional movements such that predictions of clinical scales or therapy effects can be improved), synchronized data to be collected from motion sensors (e.g., potentially adding or removing signals to be extracted from the data or adding and/or adding/removing a sensor from the exam procedure) with the most predictive value to both determine an improved motor exam, and the computational methods (e.g., if performing a lasso or elastic net regularization for linear regression one could adjust the Elastic net mixing value) with the most predictive value. This new optimized method could then be employed going forward and loaded into the motion analysis system(s) and exam procedure(s), 1406, and/or continually employed moving forward. It should be noted that this is exemplary and not all steps of FIG. 14 need to be employed depending on the patient pool, motor exam, and condition under assessment; for example, one could forego step 1403 and still develop models of prediction for certain disease states/motor exams; or steps 1401 and 1402 can be conducted on a patient pool of 10 patients, at which point the data and signal set developed from the first 10 patients can be used in steps 1403, 1404, and 1405 whereby a new optimized model and exam is developed which can be used on all some or all future patients. At any stage in the process, one could employ data imputation methods to address missing data that was potentially not recorded during a motor exam, not available or recorded in the patients clinical records or other clinical exam(s), or not available for certain patients or observations (visits), and could employ imputation methods such as regression imputation, interpolation, extrapolation, cold deck imputation and those exemplified in (Handbook of Statistical Data Editing and Imputation, T. de Waal, J Pannekoek, S Scholtus, 2011, John Wiley & Sons, Inc. ; Handbook of Missing Data Methodology, G. Molenberghs, G. Fitzmaurice, M. G. Kenward, A. Tsiatis, G. Verbeke, 2020, Chapman and Hall/CRC; Flexible Imputation of Missing Data, S van Buuren, 2018, CRC Press), the content of each of which is incorporated by reference herein in their entirety. Furthermore, while the method described is based on the motor exams, one could complement or supplement the motor exam information with other exam information (e.g., EEG exam, imaging exam, sensory exam (such as can be developed with quantitative sensory testing methods such as those described in (Occupational Neurology, Handbook of Clinical Neurology, M. Lotti, M. Bleecker, 2015, Elsevier), cognitive exam). At any stage in the process, one could employ clustering and classification methods to identify patient groups that for example manifest the disease in a specific way, progress in a certain way, and/or respond to therapy in a specific way. The patient disease classification can be used to further guide any of the steps of the process in FIG. 14 and one could do subgroup analysis at each or at specific steps based on the patient groups. The system could employ classification and clustering methods, both linear and nonlinear, such as linear discriminant analysis, k-means, fuzzy c-means, k-nearest neighbor, support vector machines, decision trees, logistic regression, and those such as exemplified in (Handbook of Statistics, Classification Pattern Recognition and Reduction of Dimensionality, P. R. Krishnaiah and L. N. Kanal, Volume 2, 1982, Elsevier; Cluster and Classification Techniques for the Biosciences, A. H. Fielding, 2007, Cambridge University Press; Handbook of Cluster Analysis, C. Hennig, M. Meila, F. Murtagh, R. Rocci, 2020 Chapman and Hall/CRC; Fuzzy Cluster Analysis: Methods for Classification, Data Analysis and Image Recognition, F. Hoppner, F. Klawonn, R. Kruse, T. Runkler, 1999, Wiley) the content of each of which is incorporated by reference herein in their entirety. For example, with a Parkinson's Disease patient, the kinematics/kinetics data gathered from a patient's motion analysis suite based exam, which could for example be with the other patient clinical data, be used to classify patients to certain disease classes or clusters, such as a tremor dominant disease, postural instability dominant disease, bradykinesia dominant disease, bradykinesia and rigidity dominant disease, a minimal tremor disease, a certain progression class, and/or a therapy responder class. As another example, the system can be used to identify disease classes currently unknown, such as for example finding subclasses of Parkinsonism with certain symptom clusters not identified previously without such a big data method we propose herein in certain embodiments or gradations of disease not typically distinguished with classic clinical exams that can be identified with the objective sensor-based methods outlined herein.

As another example of this process, which initiated at step 1401 whereby a motor exam was initiated based on 5 movements (e.g., movement 1, movement 2, movement 3, movement 4, and movement 5) at which time clinical scale 1a-c and clinical scale 2a-c were developed, which is conducted in 100 patients at monthly visits over a year with sensors: accelerometers 1-5 placed on the 5 unique body parts and an image capture device. From which, during step 1402, signals of movement were extracted (movement 1 signal a, movement 1 signal b, movement 2 signal a, movement 2 signal b, movement 3 signal a, movement 4 signal a, movement 4 signal b, movement 5 signal a, movement 5 signal b, movement 5 signal c). During step 1403 it can be demonstrated that movement 1 signal a and movement 1 signal b are redundant, and thus just one movement signal is used in the next steps. Then during step 1404 and 1405, an optimized model of prediction is developed for clinical scale 1a-c based on movement 1 signal a, movement 2 signal a, movement 2 signal b, and movement 5 signal a; clinical scale 2a-b based on movement 1 signal a, movement 2 signal a, and movement 5 signal a; and clinical scale 2c by movement 1 signal a. Overall, it is identified that an optimized motor exam can be conducted using movement 1, movement 2, and movement 5. The signals of movement that should be extracted for clinical scales 1 includes movement 1 signal a, movement 2 signal a, movement 2 signal b, and movement 5 signal a and while conducting the motor exam for this clinical scale one would only need to use the motion sensors which generate the optimally developed signals (for instance if movement 1 signal a, movement 2 signal a, movement 2 signal b, and movement 5 signal a can be gathered from accelerometer 1-2 and the image capture device one would only need to use these 3 motion sensors). This data is then feed back into the system and processed, 1406, and the full process or individual steps can be continued as desired or identified as optimal. As part of the process, one might identify classes of patients that manifest certain symptoms (for example 30 of the one hundred patients might show large changes over a year in movement 2 signal b while 70 of the patients show a stable signal in movement 2 signal b indicative of different patient classes of disease symptoms. The patient disease classification can be used to further guide any of the steps of the process in FIG. 14.

As an example of the process, the process can be initiated at step 1401 whereby a motor exam for Parkinson's Disease was initiated based on 5 movements (e.g., 10× arm flexion and extension, 10 m walk, holding hand at nose for 1 minute, 10× arm abduction and adduction, and standing prone with the eyes open for 30 seconds) at which time UPDRS3 exams were also determined, in 100 patients at monthly visits over a year while movements were assessed with sensors: accelerometers 1-5 placed on the left arm, right arm, left ankle, right ankle, and upper back, an image capture device, and a force plate as appropriate. From which, during step 1402, a number of signals of movement can be extracted such as velocity of arm movement during arm-flexion and extension, tremor power during the handheld still at the nose movement, postural stoop angle during the 10 m walk, velocity of arm movement during arm abduction and adduction, and total deviation in center of posture while standing). During step 1403 it could for example be demonstrated that movement velocity of arm movement during arm-flexion and extension and velocity of arm abduction and adduction are redundant, and thus just one movement signal is used in the next steps (Note, the movements and criteria are just theoretical examples to demonstrate the methodology herein and not indicative of actual clinical results, but examples based on Parkinson's Disease patient assessments and the process outlined herein are provided below). Then during step 1404 and 1405, an optimized model of prediction could developed for the UPDRS 3 scale based on the postural stoop angle during the 10 m walk and the movement velocity of arm movement during arm-flexion extension of the side most affected by Parkinson's Disease (Note, the movements and criteria are just theoretical examples to demonstrate the methodology herein and not indicative of actual clinical results, but examples based on Parkinson's Disease patient assessments and the process outlined herein are provided below). As part of the process the system could access data from an external database, such as for example if part of the patients initial exam completed imaging in the patient (for example a DAT scan, MRI, fMRI, EEG, CAT-Scan, PET-Scan, etc.) which was integrated as a datapoint in the statistical analysis of the patient data (e.g., DAT scan imaging data was used in conjunction with the kinematic and kinetic signals of movement to predict a patients UPDRS3, likelihood of responding to DBS, and the ideal dose of DBS for the patients) or secondarily one can further access DAT scan typical results from a local, national and/or international database repository of imaging data to further optimize the statistical analysis of patient data (e.g., DAT scan imaging data from the patient and that determined from a national database were used in conjunction with the kinematic and kinetic signals of movement to predict a patients UPDRS3, likelihood of responding to DBS, and the ideal dose of DBS for the patients). This statistical model and/or optimized statistical model is then feed back into the system and process, 1406, such that one has identified a process to predict UPDRS3 based on fewer sensors and movements than initially used Note, the movements and criteria are just theoretical examples to demonstrate the methodology herein and not indicative of actual clinical results, but examples based on Parkinson's Disease patient assessments and the process outlined herein are provided below.

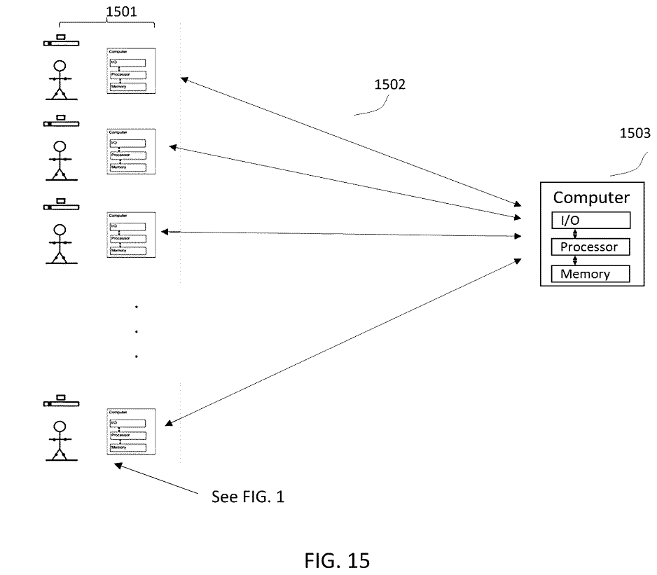

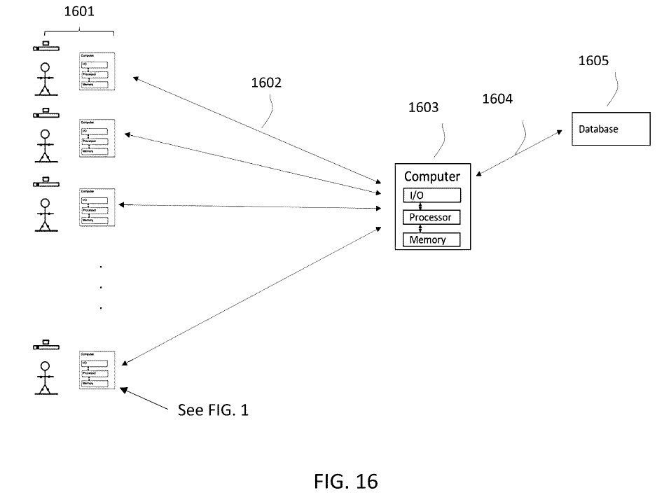

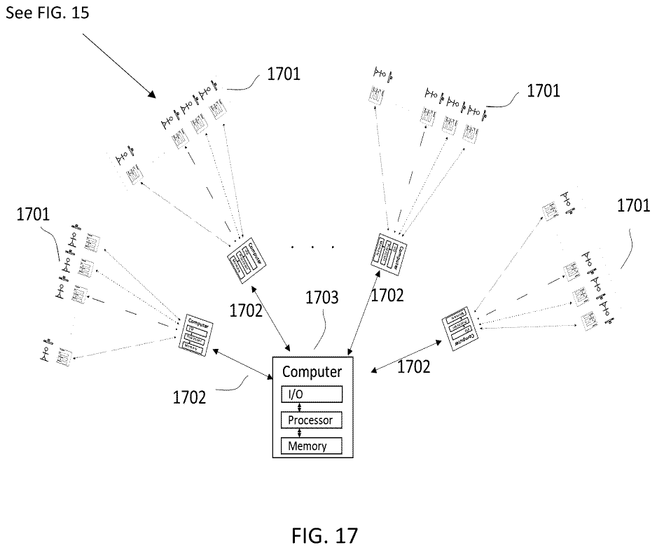

In certain embodiments, multiple systems can be connected to a central computational system so that multiple patients can be assessed simultaneously and/or at multiple locations. For example, in FIG. 15 multiple motion analysis systems are used to conduct motor exams on multiple patients, 1501. The synchronized data from motion sensors of the multiple motion analysis systems is transmitted via a connection, 1502, to a central processing system, 1503, to conduct the extraction of kinematic/kinetic signals, data reduction, statistical analysis (including prediction/inference), and optimization which can then send an optimized method back to the multiple motion analysis system, 1501, via the same connection, 1502 (or a different connection (note two individual uni-directional connections could also be employed as necessary, such as for security protocols)). The data can be compressed prior to transmitting from and/or to a sensor (e.g., camera, accelerometer) from and/or to a receiver in the CPU based system in the individual motion analysis systems from and/or to the central processor when information is communicated (either through wired or wireless communications). The data can be compressed and/or decompressed at any stage of the process, and repeated if necessary. Compression methods that can be used include examples such as Huffman coding, LZMA, or methods based on deep learning (e.g., Multi-Layer Perceptron (MLP)-based compression, Convolutional Neural Network (CNN)-based Compression, etc.) (Handbook of Data Compression, by D. Salomon, G. Motta, 2010, Springer; The Data Compression Book, M. Nelson, J. L. Gailly, 1996, BPB Publications). Such data can also be encrypted prior to transmitting and/or storing (internally in the sensor and/or at the CPU system and/or central processor). The data can be encrypted and/or de-encrypted at any stage of the process, and repeated if necessary. Encryption methods that can be used include examples such as Advanced Encryption Standard (AES), Rivest-Shamir-Adleman (RSA), Triple Data Encryption Standard (DES), Snow, Elliptic curve cryptography, Blowfish, and Twofish. Any wired or wireless communication standard in any frequency band can be used, such low energy Bluetooth, Zigbee (an IEEE 802.15.4 based specification of protocols), and passive wi-fi can be implemented. In certain embodiments, the aspects of the process can be conducted at the local system level prior to being transmitted to the central processing system, or the central processing system can be removed, and the multiple motion analysis systems can be interconnected and function as multi-processor system with multiple nodes. In certain embodiments a computer or computers that make up part of the motion analysis systems, 1501, and the central processing unit, 1503 can be used interchangeably and/or share responsibilities for addressing the data (e.g., storage, processing, transferring).

In certain embodiments, multiple systems can be connected to a central computational system so that multiple patients can be assessed simultaneously and/or at multiple locations and connected to database or databases used in any part of the process identified in FIG. 14. For example, in FIG. 16 multiple motion analysis systems are used to conduct motor exams on multiple patients, 1601. The synchronized data from motion sensors of the multiple motion analysis systems is transmitted via a connection, 1602, to a central processing system, 1603, to conduct the extraction of kinematic/kinetic signals, data reduction, statistical analysis (including prediction/inference), and optimization which can then send an optimized method back to the multiple motion analysis system, 1601, via the same connection, 1602 (or a different connection (note two individual uni-directional connections could also be employed as necessary, such as for security protocols)). The central processing system, 1603, can be connected, 1604, to a database, 1605, which contains additional patient or disease data to be used in the process. In certain, embodiments the system is connected to multiple databases. Databases could contain data such as for example data about demographics, clinical data, imaging data, any data related to daily living activities (e.g., sleep or motion data from actigraphy devices), nutrition data, daily habits data, treatment data, patient history, drug use, and data from smart wearables for tracking subject behavior. The data can be compressed and/or decompressed at any stage of the process, and repeated if necessary. Such data can also be encrypted prior to transmitting and/or storing (internally in the sensor and/or at the CPU system and/or central processor). The data can be encrypted and/or de-encrypted at any stage of the process, and repeated if necessary. Any wired or wireless communication standard in any frequency band can be used, such low energy Bluetooth, Zigbee (an IEEE 802.15.4 based specification of protocols), and passive wi-fi can be implemented. In certain embodiments the aspects of the process can be conducted at the local system level prior to being transmitted to the central processing system, or the central processing system can be removed, and the multiple motion analysis systems can be interconnected and function as multi-processor system with multiple nodes and/or to a Database or Database(s).

In certain embodiments, a single motion analysis system can be used to monitor multiple people simultaneously and predict or infer individual or group results based on scales of interest.

In certain embodiments, the system includes multiple sub nodes that aid in the computational process. For example, in FIG. 17, 1701, depicts multiple sets of motion analysis systems and processing units (such as those depicted in FIG. 15), connected, 1702, to a central processing unit, 1703. In FIG. 17, multiple motion analysis systems are used to conduct motor exams on multiple patients, part of 1701. The synchronized data from motion sensors of the multiple motion analysis systems is transmitted via a connection, to processing system(s), part of 1701, and via additional connections, 1702, to a central processing system to conduct signal segmentation, filtering, extraction of kinematic/kinetic signals/metrics, data reduction, statistical analysis (including prediction/inference), and/or optimization which can then send an optimized method back to the multiple motion analysis system, part of 1701. The processing systems, part of 1701, and/or central processing unit, 1703, can be connected to a database or databases which contain additional patient or disease data to be used in the process. Databases could contain data such as for example data about demographics, clinical data, imaging data, any data related to daily living activities (e.g., sleep or motion data from actigraphy devices), nutrition data, daily habits data, treatment data, patient history, drug use, and data from smart wearables for tracking subject behavior. The data can be compressed and/or decompressed at any stage of the process, and repeated if necessary. Such data can also be encrypted prior to transmitting and/or storing (internally in the sensor and/or at the CPU system and/or central processor). The data can be encrypted and/or de-encrypted at any stage of the process, and repeated if necessary. Any wired or wireless communication standard in any frequency band can be used, such low energy Bluetooth, Zigbee (an IEEE 802.15.4 based specification of protocols), and passive wi-fi can be implemented. In certain embodiments the aspects of the process can be conducted at the local system level prior to being transmitted to the central processing system, or the central processing system can be removed, and the multiple motion analysis systems can be interconnected and function as multi-processor system with multiple nodes and/or to a Database or Database(s).

In certain embodiments, the system or systems can further be integrated directly with a patient billing and reimbursement system(s) or database(s) to see that the motion analysis system(s) use or the use of other therapies being evaluated are properly reimbursed. For example, a motion analysis system can be employed in a patient's home setting and be used to conduct and track motor exam-based assessments on the patient. Each time the patient conducts an exam the system could communicate with a patient billing system and indicate that it had been used and ascertain the patient's insurance plan and bill accordingly to the care provider that is managing the patient and/or has prescribed the motion analysis system. For example, the system can be connected with a patient's medical record such as for example through an EPIC system, integrated with a billing system such as those used by Centers for Medicare & Medicaid Services to track and/or apply Medicare and/or Medicaid payments, through an insurance carriers billing database such as for example tracking International Classification of Disease codes and/or payments for care (e.g., such as for example Telemedicine based care). For example, the system can be integrated with a database of Current Procedural Terminology (CPT) codes, Healthcare Common Procedure Coding System (HCPCS) Codes, Durable Medical Equipment (DME) Codes, to appropriate bill for the procedure. In certain embodiments, the codes themselves can be directly integrated into the software and/or hardware of the motion analysis system and/or systems to assist the practitioner while using the motion analysis system(s).

The system, for example, can be deployed in a patient's home setting for use when a patient is prescribed a therapy and be used to help in the diagnosis of a disease (e.g., the difference of the Parkinson's Disease patient's movement patterns when on or off a L-dopa therapy and/or when undergoing a different stimulation treatment for DBS).

In certain embodiments, the system can be used in an integrated manner to dose and tune a therapeutic regimen (e.g., a neurostimulation device's frequency, current, voltage, pulse width, pulse shape, pulse timing, intensity such as those described in U.S. pat. publ. no. 2021/0322771, which can be integrated with a local and/or remote system to track and/or control the patient's therapeutic regimen.

In certain embodiments, the device can be used to provide and/or direct care, such as for example actively and/or passively controlling and/or directing a physical therapy routine that was provided to a patient. In such an example, a physical therapy routine can be provided in a standardized manner or optimized real time, based on past patient observations, based on models of disease, and/or based on models of treatment through a screen and speaker system connected to or that is part of the motion analysis suite which can be used to provide directions and/or feedback to the patients and/or care providers while conducting the physical therapy routine. For example, a stroke patient can be undergoing therapy while being tracked with a motion analysis suite's sensors and cameras, while simultaneously the patient is provided a physical therapy routine and feedback through the screen and speaker system based on assessments of the motion analysis suite of the patient's activities. In certain embodiments, this routine can be provided and/or optimized through the motion analysis suite and/or via another local system from which the feedback is provided (such as for example a motion analysis suite that implements the assessment and predictions described herein) and/or through a local operator or observer that is interfacing with the system(s) and providing patient feedback. In certain embodiments, this routine can be provided and/or optimized through a remote system where either the feedback is provided through a computer system (such as for example one that implements the assessment and predictions described herein) and/or through a remote operator or observer that is interfacing with the system(s). In addition to the patient receiving feedback, the caregiver could also be provided feedback as such as where a motion analysis suite is tracking and assessing both a patient and a caregiver, such as for example, where a physical therapist is providing manual therapy to a patient while both are being tracked and assessed. In certain embodiments, some or all of the functions can be conducted with a local computer system and some or all of functions with a remote system. Typical feedback from the motion analysis suite is based on motion analysis data, but in certain embodiments the system can be integrated with other forms of Biofeedback (from any step described herein (e.g., from observation, to analysis, to prediction and classification)) such as provided from other integrated systems of measurement and/or assessment (e.g., EEG and ERP data, EKG data, EMG, EOG, respiratory assessment, imaging assessment, metabolic assessment, electrophysiology assessment, and/or galvanic skin response) and/or systems such as those described in (Biomedical Signals, Imaging, and Informatics, J. Bronzino, D. R. Peterson, 2014, CRC Press) which is incorporated herein by reference in its entirety).

The disclosure includes methods that identify biomechanical correlations of symptoms of a movement disorder (in some cases, symptoms not normally captured by the classical clinical scales), and can use such data to tailor therapies based on specific patient biomechanical patterns, such as for example, in teaching patients specific compensatory movements based on disease patterns and/or providing brain stimulation therapies focused on specific movement patterns or providing, controlling, or dosing therapy based on specific patterns recorded during a motor exam conducted with the motion analysis suite.

Disease progression can also be tracked and/or modeled through with the systems and methods disclosed herein. For example, the system can be used to evaluate a patient at different time points and compare the change in evaluations as a function of time with evaluations based on an assessment of a single joint movement, multiple assessments of multiple movements of multiple joints, and/or an assessment of multiple correlated movements of joints which can be used to compare the patient to their earlier visits or from a model developed from data determined from using a single system and/or multiple integrated systems outlined herein. As another example a patient could come in a have their first evaluation completed with a single motion analysis suite system following a standardized assessment protocol, when once completed the patient's evaluation data can be uploaded to a cloud based system which was connected to multiple other systems used on other patient(s) in the past, present, and/or future whereby the evaluation data recorded from the patient can be used as a comparator to the other patient evaluation data set(s) and establish a baseline for tracking disease progression where future evaluations can be compared to an evaluation data sets made at subsequent time points in the other patient evaluation data set(s). As demonstrated, numerous computational methods can be used to complete such a process, and the methods described herein should be considered exemplary as one skilled in the art would note other such methods can be employed.

As discussed, tuning and/or optimizing treatment (e.g., neurostimulation, physical therapy, drug therapy) and its effects are discussed, for example in U.S. pat. publ. no. 2015/0025421. For example, the motion analysis system can be used as part of a deep brain stimulation (DBS) stimulation parameter tuning process whereby a patient undergoes an exam with a motion analysis system(s) as detailed herein to establish a baseline measure, such as for example quantifying a Parkinson's patient's baseline tremor, bradykinesia, rigidity, and/or postural instability characteristics. The patient could subsequently be provided brain stimulation via a DBS device and reassessed with the motion analysis system(s) to compare the patient's Parkinson's patient's during stimulation tremor, bradykinesia, rigidity, and/or postural instability characteristics to the baseline characteristics. Furthermore, the practitioner could vary the DBS stimulation parameters (e.g., voltage, current, pulse frequency, pulse width, pulse shape, electrode lead, polarity) and assess the change in the data from the motion analysis system(s) to determine the stimulation parameters which improve the patient's symptoms. One could also use the process to selectively tune the patient's response, such as for example maximally improving a patient's postural instability, while potentially having less effect on other symptoms (e.g., tremor) such as for example in a patient that had a history of falls. As another example, a set of network connected motion analysis suite(s) can be used with multiple patients, either in discrete or ongoing evaluations, following the exemplified tuning process, and a central computation system could evaluate this discrete or expanding data set via the statistical/AI based methods described herein and/or the incorporated references to tune the stimulation patterns. A big data approach and/or an adaptive model approach can be implemented where ongoing evaluations from large numbers of patients can be continually implemented to continually improve the stimulation tuning. Such a method can be integrated with other patient data sets to further optimize the stimulation (e.g., EEG, MRI, EKG, patient history, behavioral assessments, clinical exam data, cognitive assessments). Such as for example, numerous motion analysis systems can be connected via the internet but deployed to multiple clinical sites where stroke patients are assessed and provided physical/rehabilitation therapy. The connected systems could initially assess a patients baseline information and be used to collect additional data, such as patient imaging data and/or clinical assessments, which can be uploaded to a central system which develops mathematical models, such as a linear model or correlation models, described the relationship between the patient's motor exam values gathered with the motion analysis systems and the clinical assessments and/or imaging data. The uploaded data or generated models can further be used to classify patients (and/or potentially identify patients that would best respond to specific physical therapy/rehabilitation regimens). As more patients are evaluated and/or the patient(s) or patients begin and/or continue to undergo treatment, further data can be uploaded to the central system and the classification and/or therapy tuning can further be improved and optimized. Furthermore, as the classification and/or treatment can be influenced by this motion analysis system(s) based exams, such as by using feedback, one can continuously optimize the classification and/or treatment process by providing the feedback to those conducting the motion analysis system-based exams. The multiple motion analysis systems can be connected to a central computation system, or the connected multiple motion analysis systems can work in parallel to complete the computational processes. As demonstrated, numerous computational methods can be used to complete such a process, and the methods described herein should be considered exemplary as one skilled in the art would note other such methods can be employed.

### Big Data Integration

Herein we provide additional description of the way in which big data can be used with brain stimulation, neuromodulation, motion analysis suite(s), cost effective analysis software, diagnostics, prognostics, health care and/or combined elements (e.g., Big Data Application of a Personalized Therapy Suite and the Associated Elements) such as for example with a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment. Big data's status and current impact in the medical and basic sciences, such as those in Basic Neurosciences, Neurology, Pain Medicine, Addiction Medicine, and Rehabilitation Medicine, exemplified through work done in areas such as Connectomics, Alzheimer's Disease, Stroke, Depression, Parkinson's Disease, Pain, and Addiction can be advanced in combination with the methodologies exemplified in this application.

As examples, neuroscience subfields are implementing big data approaches, such as computational neuroscience (Trappenberg, Fundamentals of Computational Neuroscience, 2010), neuroelectrophysiology (Chung et al., High-density single-unit human cortical recordings using the Neuropixels probe, Neuron, 2022; Ikegaya et al., Synfire chains and cortical songs: temporal modules of cortical activity, Science, 2004; Pnevmatikakis et al., Simultaneous Denoising, Deconvolution, and Demixing of Calcium Imaging Data, Neuron, 2016; Reed & Kaas, Statistical analysis of large-scale neuronal recording data, Neural Netw, 2010), and connectomics (Scheffer et al., A connectome and analysis of the adult Drosophila central brain, Elife, 2020) to elucidate the structure and function of the brain and can be used for improving noninvasive brain stimulation treatments, motion analysis suite(s), cost effective analysis software, diagnostics, prognostics, health care and/or combined elements. Databases and data such as those exemplified in (Dipietro L, Gonzalez-Mego P, Ramos-Estebanez C, Zukowski L H, Mikkilineni R, Rushmore R J, Wagner T. The evolution of Big Data in neuroscience and neurology. J Big Data. 2023;10(1):116. doi: 10.1186/s40537-023-00751-2. Epub 2023 Jul. 10. PMID: 37441339; PMCID: PMC10333390) can be integrated with these methods and/or data exemplified in this section and the rest of the document.

Such methods could for example be interfaced with brain stimulation dosing software (such as those described in U.S. pat. publ. no. 2021/0322771, incorporated herein in its entirety), such as for by integrating the neural structure as demonstrated by a connectomic(s) approaches with neuroelectrophysiology data to predict or guide the stimulation doses based on a desired outcome from the stimulated tissue(s). Neuroelectrophysiology techniques, such as where simultaneous recordings made from single to hundreds to thousands to hundreds of thousands to millions to billions to trillions of brain neurons (Chung et al., High-density single-unit human cortical recordings using the Neuropixels probe, Neuron, 2022; Ikegaya et al., Synfire chains and cortical songs: temporal modules of cortical activity, Science, 2004; Pnevmatikakis et al., Simultaneous Denoising, Deconvolution, and Demixing of Calcium Imaging Data, Neuron, 2016; Reed & Kaas, Statistical analysis of large-scale neuronal recording data, Neural Netw, 2010) necessitate big data techniques to decode the electrical signals of the brain can be coupled with the examples described (and/or for combinations from multiple recordings from multiple cells, nerves, nervous systems, and/or brains); and connectomonics, which can implement ultrahigh resolution histological imaging methods, such as electron microscopy, to allow for complete reconstructions of structures at submicron resolution (Scheffer et al., A connectome and analysis of the adult Drosophila central brain, Elife, 2020) can be coupled as an imaging method with brain stimulation, neuromodulation, motion analysis suite(s), cost effective analysis software, diagnostics, prognostics, health care and/or combined elements to optimize the techniques. Currently, Neurology initiatives commonly use large, highly heterogeneous data sets (e.g., neuroimaging, genetic testing, and/or clinical assessments from 1,000 to 10,000s+ patient groups (Bethlehem et al., Brain charts for the human lifespan, Nature, 2022; Demro et al., The psychosis human connectome project: An overview, Neuroimage, 2021; Drysdale et al., Resting-state connectivity biomarkers define neurophysiological subtypes of depression, Nat Med, 2017; Kim et al., Scaling Up Research on Drug Abuse and Addiction Through Social Media Big Data, J Med Internet Res, 2017; Veitch et al., Using the Alzheimer's Disease Neuroimaging Initiative to improve early detection, diagnosis, and treatment of Alzheimer's disease, Alzheimers Dement, 2022; Xia et al., Connectome gradient dysfunction in major depression and its association with gene expression profiles and treatment outcomes, Mol Psychiatry, 2022)) and by acquiring big data with increasing velocity (e.g., using real-time wearable sensors (Pnevmatikakis et al., Simultaneous Denoising, Deconvolution, and Demixing of Calcium Imaging Data, Neuron, 2016)) with or without technologies adapted from other fields (e.g., automatized clinical note assessment (Wheatley, Google's latest AI tools help doctors read medical records faster, 2020), social media-based infoveillance applications (Kim et al., Scaling Up Research on Drug Abuse and Addiction Through Social Media Big Data, J Med Internet Res, 2017; Nasralah et al., Social Media Text Mining Framework for Drug Abuse: Development and Validation Study With an Opioid Crisis Case Analysis, J Med Internet Res, 2020)) one could effectively improve brain stimulation, neuromodulation, motion analysis suite(s), cost effective analysis software, diagnostics, prognostics, health care and/or the combined methods to optimize the techniques.

Big Data Connectomes: The human brain contains ˜100 billion neurons that are connected through ˜10¹⁴ synapses, through which electrochemical data is transmitted (Briscoe & Marin, Looking at neurodevelopment through a big data lens, Science, 2020). Neurons are organized into discrete regions or nuclei and connect in precise and specific ways to neurons in other regions; the aggregated connections between all neurons in an individual comprises their connectome. The connectome is a large and complex dataset characterized by tremendous interindividual variability (Sporns et al., The human connectome: A structural description of the human brain, PLoS Comput Biol, 2005). Connectomes, at the level of the individual or as aggregate data from many individuals have the potential to produce a better understanding of how brains are wired as well as to unravel the “basic network causes of brain diseases” for prevention and treatment (Abbott, How the world's biggest brain maps could transform neuroscience, Nature, 2021; Nair, Connectome, Proc Natl Acad Sci USA, 2013; Sporns, The human connectome: a complex network, Ann NY Acad Sci, 2011; Sporns et al., The human connectome: A structural description of the human brain, PLoS Comput Biol, 2005). The connectome can be used as the basis of dose-based modeling and targeting, where one can align the connectome information with dosing software for brain stimulation. Exemplary embodiments of the apparatuses and methods disclosed can be employed in the area of analyzing, predicting, controlling, and optimizing the dose of energy for neural stimulation, for directly stimulating neurons, depolarizing neurons, hyperpolarizing neurons, modifying neural membrane potentials, altering the level of neural cell excitability, and/or altering the likelihood of a neural cell firing (during and after the period of stimulation). This for example can be used to alter brain oscillations. Exemplary apparatuses for stimulating tissue are described for example in Wagner et al., (U. S pat. publ. nos. 2008/0046053 and 2010/0070006), the content of each of which is incorporated by reference herein in its entirety. Likewise, methods for stimulating biological tissue may also be employed in the area of muscular stimulation, including cardiac stimulation, where amplified, focused, direction altered, and/or attenuated currents can be used to alter muscular activity via direct stimulation, depolarizing muscle cells, hyperpolarizing muscle cells, modifying membrane potentials, altering the level of muscle cell excitability, and/or altering the likelihood of cell firing (during and after the period of stimulation). Likewise, methods for stimulating tissue can be used in the area of cellular metabolism, physical therapy, drug delivery, and gene therapy. Furthermore, stimulation methods described herein can result in or influence tissue growth (such as promoting bone growth or interfering with a tumor). Furthermore, devices and methods can be used to solely calculate the dose of the fields, for non-stimulatory purposes, such as assessing the safety criteria such as field strengths in a tissue (such as for example delivering energy to treat a potential brain cancer). The embodiments outlined herein for calculating, controlling, tuning, and/or optimizing energy doses of stimulation can be integrated (either through feedback control methods or passive monitoring methods) with imaging modalities, physiological monitoring methods/devices, diagnostic methods/devices, and biofeedback methods/devices (such as those described in co-owned and co-pending U.S. pat. publ. no. 2011/0275927, the content of which is incorporated by reference herein in its entirety). The embodiments outlined herein for calculating/controlling energy doses of stimulation can be integrated with or used to control the stimulation source properties (such as number, material properties, position (e.g., location and/or orientation relative to tissue to be stimulated and/or other sources or components to be used in the stimulation procedure) and/or geometry (e.g., size and/or shape relative to tissue to be stimulated and/or other sources or components to be used in the stimulation procedure)), the stimulation energy waveform (such as temporal behavior and duration of application), properties of interface components (such as those outlined in U.S. pat. publ. no. 2010/0070006) and, for example, position, geometry, and/or material properties of the interface materials), and/or properties of focusing or targeting elements (such as those outlined in (co-owned and co-pending U.S. pat. publ. no. 2011/0275963, the content of which is incorporated by reference herein in its entirety) and for example position, geometry, and/or material properties of the interface materials) used during stimulation. See also, U.S. pat. publ. no. 201/0228716, the disclosure of which is hereby incorporated herein in its entirety. The dose of energy(ies) can include the magnitude, position, dynamic behavior (i.e., behavior as a function of time), static behavior, behavior in the frequency domain, phase information, orientation/direction of energy fields (i.e., vector behavior), duration of energy application (in single or multiple sessions), type/amount/composition of energy (such as for electromagnetic energy, the energy stored in the electric field, the magnetic field, or the dissipative current component (such as can be described with a Poynting Vector)), and/or the relationship between multiple energy types (e.g., magnitude, timing, phase, frequency, direction, and/or duration relationship between different energy types (such as for example for an electromechanical energy (i.e., energy provided from mechanical field source, such as ultrasound device, and an electrical field source, such as an electrode) pulse, the amount of energy stored in an acoustic energy pulse compared with that stored in an electric pulse)). Dose of energy may be analyzed, controlled, tuned, and/or optimized for its impact on a cell, tissue, functional network of cells, and/or systemic effects of an organism. The connectome and big data approaches can be used to optimize this approach, such as integrating the connectome, brain stimulation field data, and/or an AI based algorithm (see above for examples) to tune the dose delivery in a manner which would best impact the patient. This can be done on an individual patient basis, or across large patient groups and datasets. Furthermore, this can be integrated with other data, such as that described for use in the motion analysis system to further tune the data (such as for example identifying a brain connection and motion analysis recorded movement pattern phenotype for optimizing therapy as exemplified above). The term tissue filtering properties refer to anatomy of the tissue(s) (e.g., distribution and location), electromagnetic properties of the tissue(s), cellular distribution in the tissue(s) (e.g., number, orientation, type, relative locations), mechanical properties of the tissue(s), thermodynamic properties of the tissue(s), chemical distributions in the tissue(s) (such as distribution of macromolecules and/or charged particles in a tissue), chemical properties of the tissue(s) (such as how the tissue effects the speed of a reaction in a tissue), and/or optical properties of the tissue(s) which has a temporal, frequency, spatial, phase, and direction altering effect on the applied energy. The term filtering includes the reshaping of the energy dose in time, amplitude, frequency, phase, type/amount/composition of energy, or position, or vector orientation of energy (in addition to frequency dependent anisotropic effects). Filtering can result from a number of material properties that act on the energy, for example this includes a tissue's (and/or group of tissues'): impedance to energy (e.g., electromagnetic, mechanical, thermal, optical, etc.), impedance to energy as a function of energy frequency, impedance to energy as a function of energy direction/orientation (i.e., vector behavior), impedance to energy as a function of tissue position and/or tissue type, impedance to energy as a function of energy phase, impedance to energy as a function of energy temporal behavior, impedance to energy as a function of other energy type applied and/or the characteristics of the other energy type (such as for a combined energy application where an additional energy type(s) is applied to modify the impedance of one tissue relative to other energy types that are applied), impedance to energy as function of tissue velocity (for tissue(s) moving relative to the energy and/or the surrounding tissue(s) moving relative to a targeted tissue), impedance to energy as a function of tissue temperature, impedance to energy as a function of physiological processes ongoing in tissue(s), impedance to energy as a function of pathological processes ongoing in tissue(s), and/or impedance to energy as a function of applied chemicals (applied directly or systemically). One could use the connectome data to directly generate an impedance model of the targeted tissue (by using impedance values of the cells and or tissue that make up the connectome to calculate the network impedance (see for example FIG. 1.3 of (Wagner, T. (2006). Noninvasive brain stimulation: modeling and experimental analysis of transcranial magnetic stimulations and transcranial DC stimulations as a modality for neuropathology treatment. MIT. HST PhD Thesis. Cambridge, MA.) for how one could use the tissues, or see the references incorporated herein for other examples (e.g., 2021/0322771 Methods of Stimulating Tissue Based Upon Filtering Properties of the tissue or an averaged impedance value typical of the targeted site to optimize the energy delivered to the targeted cells. Filtering can further be caused by the relationship between individual impedance properties to an energy or energies (such as for example the relationship that electrical conductivity, electrical permittivity, and/or electrical permeability have to each other). This can further include the velocity of propagation of energy in the tissue(s), phase velocity of energy in the tissue(s), group velocity of energy in the tissue(s), reflection properties to energy of the tissue(s), refraction properties to energy of the tissue(s), scattering properties to energy of the tissue(s), diffraction properties to energy of the tissue(s), interference properties to energy of the tissue(s), absorption properties to energy of the tissue(s), attenuation properties to energy of the tissue(s), birefringence properties to energy of the tissue(s), and refractive properties to energy of the tissue(s). This can further include a tissue(s'): charge density (e.g., free, paired, ionic, etc.), conductivity to energy, fluid content, ionic concentrations, electrical permittivity, electrical conductivity, electrical capacitance, electrical inductance, magnetic permeability, inductive properties, resistive properties, capacitive properties, impedance properties, elasticity properties, stress properties, strain properties, combined properties to multiple energy types (e.g., electroacoustic properties, electrothermal properties, electrochemical properties, etc.), piezoelectric properties, piezoceramic properties, condensation properties, magnetic properties, stiffness properties, viscosity properties, gyrotropic properties, uniaxial properties, anisotropic properties, bianisotropic properties, chiral properties, solid state properties, optical properties, ferroelectric properties, ferroelastic properties, density, compressibility properties, kinematic viscosity properties, specific heat properties, Reynolds number, Rayleigh number, Damkohler number, Brinkman number, Nusselt Schmidt number, number, Peclet number, bulk modulus, Young's modulus, Poisson's ratio, Shear Modulus, Prandtl number, Adiabatic bulk modulus, entropy, enthalpy, pressure, heat transfer coefficient, heat capacity, friction coefficients, diffusivity, porosity, mechanical permeability, temperature, thermal conductivity, weight, dimensions, position, velocity, acceleration, shape, convexity mass, molecular concentration, acoustic diffusivity, and/or coefficient of nonlinearity. These values can further be integrated into a connectome model, either based on the cell and tissue distribution of the targeted tissue, or based on an average model developed across a population of connectomes. Filtering can occur at multiple levels in the processes. For example, with multiple energy types filtering can occur with the individual energies, independent of each other (such as where acoustic and electrical energy are applied to the tissue at separate locations and the fields are not interacting at the sites of application), and then filtering can occur on the combined energies (such as where acoustic and electrical energy interact in a targeted region of tissue). Furthermore, any material and/or sub-property in a focusing element, interface element, and/or component(s) of the energy source element that can actively or passively alter the energy field properties of stimulation can also be accounted for in the dosing procedures explained herein (including any space, fluid, gel, paste, and material that exists between the tissue to be stimulated and the stimulation energy source). For example, methods of the disclosure can also account for: lenses (of any type (e.g., optical, electromagnetic, electrical, magnetic, acoustic, thermal, chemical, etc.)); using waveguides; using fiber optics; phase matching between materials; impedance matching between materials; using reflection, refraction, diffraction, interference, and/or scattering methods between materials. As described above numerous assessment methods (e.g., EEG, MRI, EKG, patient history, behavioral assessments, clinical exam data, cognitive assessments) can be integrated with the methodology for optimization (see the references incorporated herein for further examples).

Further big data advancements in elucidating the connectome (see for example table 3 and supplementary table 3 of (Dipietro L, Gonzalez-Mego P, Ramos-Estebanez C, Zukowski L H, Mikkilineni R, Rushmore R J, Wagner T. The evolution of Big Data in neuroscience and neurology. J Big Data. 2023;10(1):116. doi: 10.1186/s 40537-023-00751-2. Epub 2023 Jul. 10. PMID: 37441339; PMCID: PMC10333390) can be integrated with the methods exemplified herein (e.g., neuroimaging techniques and data like Magnetic Resonance Imaging (MRI) and Diffusion Tensor Imaging (DTI) data can be used to generate anatomical connectomes and neuroimaging techniques such as functional MRI (fMRI), to generate functional connectomes (Elam et al., The Human Connectome Project: A retrospective, Neuroimage, 2021; Li et al., Functional Neuroimaging in the New Era of Big Data, Genomics Proteomics Bioinformatics, 2019) can integrated with brain stimulation, neuromodulation, motion analysis suite(s), cost effective analysis software, diagnostics, prognostics, health care and/or combined elements such as for example with a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment). For example, work exemplified by Bethlehem et. al.'s study for developing “Brain charts for the human lifespan” which was based on aggregated 123,984 MRI scans, across more than 100 primary studies, from 101,457 human participants between 115 days post-conception to 100 years of age (Bethlehem et al., Brain charts for the human lifespan, Nature, 2022), can be used to appropriately provide a brain stimulation dose to a patient based on their age, or a patients age guided connectome can be coupled with a motion analysis suite based data set to appropriately optimize a patients treatment plan. Connectomes of patient cohorts can also be used in this manner (i.e., either a single patient or a group of patients). Neuroimaging phenotypes and developmental trajectories, as developed via MRI imaging, can thus be integrated into the methodologies described in this document including the brain stimulation technology and/or motion analysis suite software.

In addition to generating datasets with increasingly larger volume, the human connectome studies are also characterized by highly heterogeneous datasets, which stem from the use of multimodal imaging. For example, studies conducted under the Human Connectome Project (“HCP”) (Human Connectome Project: What is the Connectome Coordination Facility?, 2011) have implemented structural MRI, resting-state fMRI (rsfMRI), task fMRI (tfMRI), and diffusion MRI (dMRI) imaging modalities, with subsets undergoing Magnetoencephalography (MEG) and Electroencephalography (EEG). HCP studies are generally based on datasets of 100′s-1000+ subjects (e.g., Healthy Adult (1100 healthy young adults), HCP Lifespan Studies (e.g., HCP Aging 1200 healthy adults aged 36-100+)), which are often coupled with additional data such as clinical assessments or biospecimens (HCP, What is the Connectome Coordination Facility?), can further be coupled with brain stimulation, neuromodulation, motion analysis suite(s), cost effective analysis software, diagnostics, prognostics, health care and/or combined elements such as for example with a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment.

Big Data Genetics and/or Imaging: Genetic information and/or analysis methods can be used to optimize therapy such as can be used with brain stimulation, neuromodulation, motion analysis suite(s), cost effective analysis software, diagnostics, prognostics, health care and/or combined elements such as for example with a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment. For example, using biomarkers for early detection and progression tracking of Alzheimer's disease (AD), as well as for also for monitoring effects of AD therapeutics and facilitating development of new treatments (Mueller et al., The Alzheimer's disease neuroimaging initiative, Neuroimaging Clin N Am, 2005; Weiner et al., The Alzheimer's disease neuroimaging initiative: progress report and future plans, Alzheimers Dement, 2010) can be coupled with brain stimulation and/or motion analysis systems to improve their operation (e.g., altering a brain stimulation dose based on a patient genotype or phenotype (such as incorporating the genetic based response data or genetics/connectome combined data for patient treatment dosing) or coupling a motion analysis suite based examination with genetic information to aid in diagnosis or prognosis of a patient). Furthermore, one could couple genetics and imaging information together to be used with brain stimulation, neuromodulation, motion analysis suite(s), cost effective analysis software, diagnostics, prognostics, health care and/or combined elements such as for example with a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment. For example, one could employ the methods and/or data set of the Enhancing Neuroimaging Genetics through Meta-analysis (ENIGMA) Consortium (Bearden & Thompson, Emerging Global Initiatives in Neurogenetics: The Enhancing Neuroimaging Genetics through Meta-analysis (ENIGMA) Consortium, Neuron, 2017; Thompson et al., The Enhancing NeuroImaging Genetics through Meta-Analysis Consortium: 10 Years of Global Collaborations in Human Brain Mapping, Hum Brain Mapp, 2022) to identify phenotypes for potential therapy types, or optimal doses of treatment (e.g., brain stimulation), which can further be coupled with the motion analysis methods described herein to optimize a brain stimulation treatment and physical therapy session (such as for example as a method to improve a patients training of a physical task, such as improving a patients balance to reduce fall risk). For example, one could conduct or use the data from large scale MRI studies in specific pathologies that are known to affect balance and show imaging-based abnormalities and/or structural changes that can be targeted with brain stimulation and/or serve as an additional basis of information for the motion analysis suite directed physical therapy for balance training. This can be used in any number of indications, beyond those that affect balance, such as those exemplified in the references incorporated herein and as those exemplified in THE EVOLUTION OF BIG DATA IN NEUROSCIENCE AND NEUROLOGY incorporated hereinabove, and general conditions impacting a patient's health (e.g., cardiovascular, endocrine, and/or pulmonary ailments). Other genetics/imaging-based datasets which demonstrate exemplary data and methodologies that can be used include genome-wide association studies of UK Biobank (Smith et al., An expanded set of genome-wide association studies of brain imaging phenotypes in UK Biobank, Nat Neurosci, 2021; Sun et al., Genetic map of regional sulcal morphology in the human brain from UK biobank data, Nat Commun, 2022; Zhao et al., Genome-wide association analysis of 19,629 individuals identifies variants influencing regional brain volumes and refines their genetic co-architecture with cognitive and mental health traits, Nat Genet, 2019), Japan's Brain/MINDS work (Okano et al., Brain/MINDS: A Japanese National Brain Project for Marmoset Neuroscience, Neuron, 2016), and the Brainstorm Consortium (Brainstorm et al., Analysis of shared heritability in common disorders of the brain, Science, 2018). For example, the Brainstorm Consortium assessed “25 brain disorders from genome-wide association studies of 265,218 patients and 784,643 control participants and assessed their relationship to 17 phenotypes from 1,191,588 individuals.” Big data-based genetic and imaging assessments such as in in the neurology space can be coupled with brain stimulation methods (e.g., diagnostic testing from baseline patient testing or therapeutic response data to brain stimulation patient treatment) and/or coupled with the motion analysis system methods or analysis results to improve therapy, such as in defining more refined disease phenotypes or therapeutic response characteristics (e.g., patient disease classes with particular diagnostic and/prognostic value) to a particular dose of treatment. The methods can be used to identify common clinical risk factors for disease, such as gender, age, and geographic location (and/or its genetic and/or imaging-based risk factors).

“Real Time” Big Data Examples: Additionally, big data approaches of combing high volumes of varied data at high velocities are offering the potential for new “real-time” biomarkers (McCarthy, The Biomarker Future is Digital, Inside Precision Medicine, 2020), which would be useful in brain stimulation, neuromodulation, motion analysis suite(s), cost effective analysis software, diagnostics, prognostics, health care and/or combined elements such as for example with a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment. For example, data collected with wearable sensors have been increasingly used in clinical studies to monitor neuro-patient behavior at home or in real-world settings. While the classic example is the use of EEG for epilepsy (Kiral-Kornek et al., Epileptic Seizure Prediction Using Big Data and Deep Learning: Toward a Mobile System, EBioMedicine, 2018), numerous other embodiments can be found in the literature. For example, another approach, involves the use of smartphone data to evaluate the feasibility of collecting information on daily changes in symptom severity and sensitivity to medication in PD patients (Bot et al., The mPower study, Parkinson disease mobile data collected using ResearchKit, Sci Data, 2016), which can be integrated with the methodologies outlined above to optimize a patients therapy through numerous integrated motion analysis suites (or via numerous patients analyzed through a central server via network integration). This for example can be coupled with a brain stimulation system. The motion analysis suite system could also be used to improve randomized controlled trial (RCT) design to address: cost, time to complete, generalizability of results, and limited observations (e.g., made at a limited number of predefined time points in a protocol (e.g., baseline, end of treatment)). Standardization and automation of procedures using big data make entering and extracting data easier and can be used to reduce effort and cost to run a RCT as can be fostered through the motion analysis suite(s) as the backbone of the analysis. They can also be used to formulate hypotheses fueled by large, preliminary observational studies (such as the motion analysis suite(s) deployed to many Parkinsonian patients' homes for real-time analysis and data gathered from the real time assessments can be used carry out virtual trials (and/or optimize a larger trial design such as coupled with cost effectiveness software). Big data, such as using data that can be gathered from Electronic Health Records (EHRs), pharmacy dispensing, and payor records, can be coupled with the motion analysis system(s) to help evaluate the safety and efficacy of therapeutics. Crowdsourcing of data acquisition and analysis via the motion analysis suite(s) and/or other assessment methods exemplified herein can be used to grow a data set and ultimately aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment. Social media can also be used to monitor patient behavior and potential responses to therapy, which can be integrated with the motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment.

Big Data “Value” Optimization: Big data studies, such as with the motion analysis suite(s), can be coupled with Health Economics methods and assign more quantitative valuations to data sets (Rafferty et al., Cost-Effectiveness Analysis to Inform Randomized Controlled Trial Design in Chronic Pain Research: Methods for Guiding Decisions on the Addition of a Run-In Period, Princ Pract Clin Res, 2022). These methods can be used in brain stimulation, neuromodulation, motion analysis suite(s), diagnostics, prognostics, health care and/or combined elements such as for example with a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment. Health Economics methods include software and computational based methods for determining an optimized design or cost-effective design for a clinical trial, such as a Randomized Controlled Trial (RCT) for evaluating medical therapies and/or for optimizing a patient's therapy.

Pharmacoeconomic methods and/or decision-support techniques are employed to guide decisions that maximize limited resources with the highest value to patients, providers, payers, and society for the evaluation health effects of RCTs. Methods employed include cost-benefit analysis (CBA), cost-effectiveness analysis (CEA), cost-utility analysis (CUA), and others. Such methods are becoming more common place in the practice of medicine, for example cost effectiveness assessments are integral to National Institute for Health and Care Excellence (NICE) Guidelines [https://www.nice.org.uk/process/pmg6/chapter/assessing-cost-effectiveness]. CEA defines health benefits in natural units, costs in monetary units, and compares health gains of medical procedures via the same outcome measures.

An example generally relates to a system, software, and/or computational based methods for determining an optimized design for randomized controlled trial (RCT) for evaluating medical therapies (which can be incorporated with the motion analysis suite(s) and/or brain stimulation methods). Further embodiments of the system, software, and computational based methods can be used as a decision support tool for scenario comparisons and/or mission planning across other fields beyond healthcare.

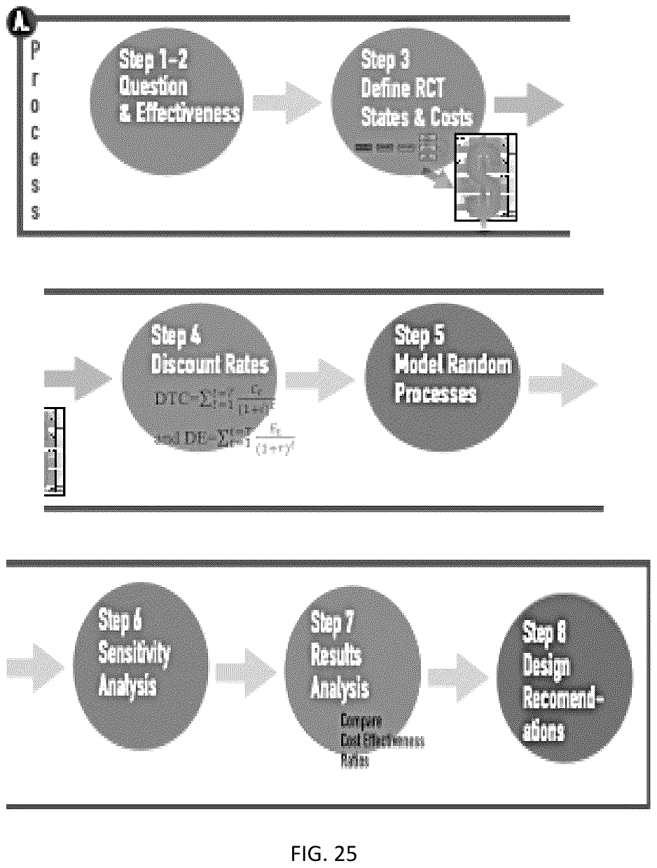

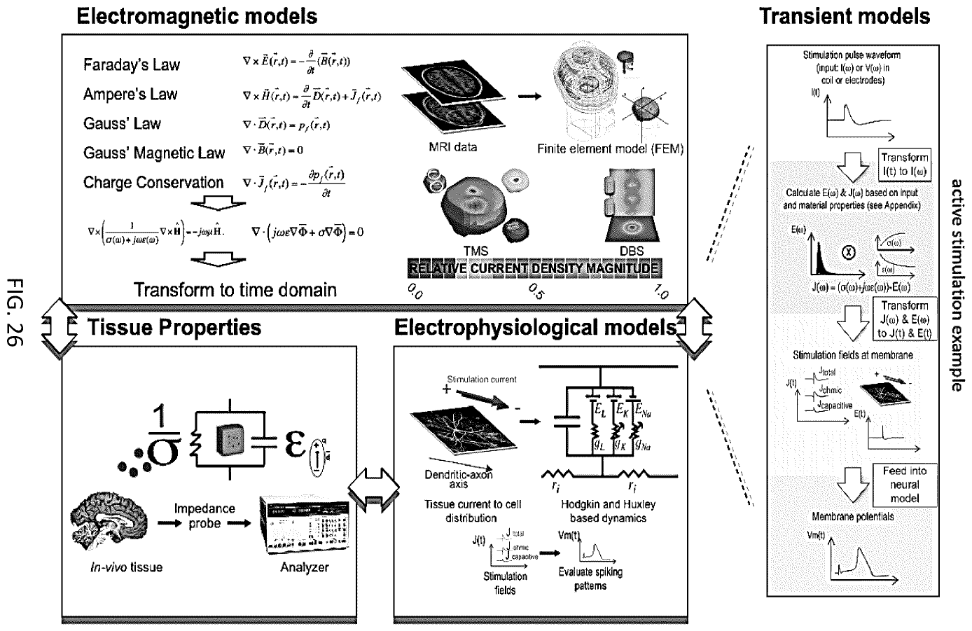

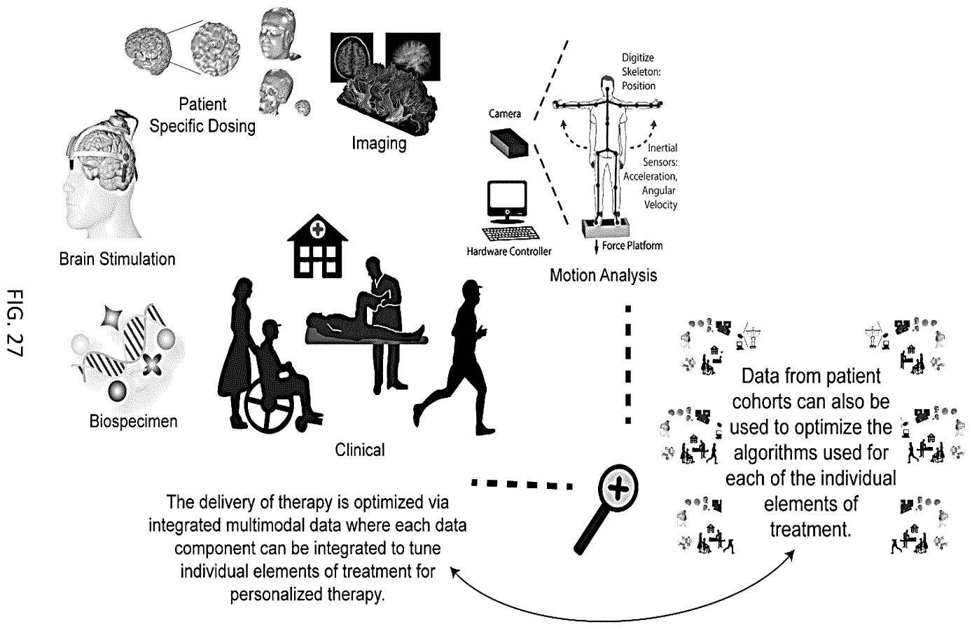

The example generally relates to a system, methods, and/or software for optimizing an RCT design for maximum cost effectiveness. Generally, the disclosure includes software and computational based methods for a CEA Based RCT Design, focused on: 1. Defining the research question 2. Defining effectiveness; 3. Identifying the RCT States and costs; 4. Discounting (cost and effectiveness); 5. Modeling the stochastic nature of the RCT; 6. Performing a sensitivity analysis; 7. Analyzing Results; and 8. Recommending design (see FIG. 25). These steps can be employed as a whole or in part. The methods can be employed in advance to design an RCT, during the RCT to improve it, and/or after an RCT to better optimize the RCT design, evaluate past RCTs, and/or to design future RCTs.

The methods, and/or software can be implemented on any computational device and be administered via any computational device such as directly via the device, via a network (e.g., external device(s)), and/or cloud-based computing. The software is designed such that the RCT Design variables can be entered via data entry methods, such as into the computational process directly (such as through a keyboard, voice input, and/or a touch screen system), via external software (e.g., Matlab, Excel, database software (census data, data from internet)), and/or via external files (e.g., electronic text files). The RCT Design variables can include any design variable that can be altered in the design of an RCT, including but not limited to the population size, duration of trial and/or individual phases, states, and/or individual elements and/or procedures, cost of RCT elements and/or individual phases, states, and/or individual elements and/or procedures, number of personnel, skill set of personnel, advertising used for recruitment, equipment available, institution properties (e.g., number, size, resources, geographical location), potential patient qualities (e.g., adherence and compliance properties, gender, age, number, degree and rate of disability), expected treatment effects (e.g., size, duration, side effects, outcome measures), and/or analysis methods (e.g., computational methods). The RCT design can be based on any definition of effectiveness and/or an inventory of effectiveness criteria, which can include elements of the RCT Design variables. The results analysis can be focused on any standard way of reporting RCT design data and cost effectiveness data (e.g., discounted costs, discounted effectiveness, cost effectiveness ratios (CERs), incremental cost effectiveness ratios (ICERs), tornado diagrams, cost-effectiveness planes) and can be based on discrete, a range of results or probabilistic report of data. The design recommendations can be based on post design analysis or real-time alteration of RCT design criteria, and give a discrete or probabilistic report of recommendations. Elements of the results analysis and other software modules can be found in references including: (Jean-Michel Josselin and Benoît Le Maux “Statistical Tools for Program Evaluation: Methods and Applications to Economic Policy, Public Health, and Education”); (MIT Critical Data “Secondary Analysis of Electronic Health Records”); (“WHO GUIDE TO COST-EFFECTIVENESS ANALYSIS”); (www.communities.gov.uk “Multi-criteria analysis: a manual”); (“Markov Models in Medical Decision Making: A Practical Guide” FRANK A. SONNENBERG, MD, J. ROBERT BECK, MD); (THE GREEN BOOK CENTRAL GOVERNMENT GUIDANCE ON APPRAISAL AND EVALUATION); (“Handbook of Markov Chain Monte Carlo”); (“Average Cost-Effectiveness Ratio with Censored Data” Heejung Bang and Hongwei Zhao); (“Medical Decision Making” Harold C. Sox, Michael C. Higgins and Douglas K. Owens); (“Cost Effectiveness Analysis in Health A Practical Approach” Peter Muennig); (“Overview of Cost-effectiveness Analysis” Gillian D. Sanders, Ph. D; Matthew L. Maciejewski, Ph. D; Anirban Basu, Ph. D).

Software and computational modules can effectively work via first defining the question (goal of trial) and metric that will be used to assess the effectiveness of the trial design, this can include number of patient observations and/or type completed, RCT study power, cost limits, screening criteria, levels of statistical significance of observed treatment, efficacy goal of treatment, and/or their combination. Computationally one can use this to establish criteria to evaluate and design the trial, such as in the additional paper included herein (in additional files section), or via computational methods such as in (Jean-Michel Josselin and Benoît Le Maux “Statistical Tools for Program Evaluation: Methods and Applications to Economic Policy, Public Health, and Education”); (MIT Critical Data “Secondary Analysis of Electronic Health Records”); (“Fundamentals of Biostatistics” Bernard Rosner). These can be programmed in standard programming languages and implemented via standard programming methods and/or implemented via standard computational methods. Following the identification of the Question and Effectiveness Measure, one will enter variables of the RCT design into the computational system or as variables in the software used to conduct the program. Variables could include any aspect of the RCT designs being evaluated and compared (e.g., institution number, cost elements, time durations). These variables can be entered as discrete base values, as a range of variables, equations, with/without confidence intervals, and/or as a probability distribution. During this stage one enters all of the variables associated with the RCT, the phases and states and the way in which they are connected, the costs and durations of the states and phases, and the way in which they are connected. One can do this for every base scenario they want to analyze and compare. Next one will define the way in which the costs and measures of effectiveness are discounted, these can be defined in any typical manner discounting is employed and can be entered as discrete base values, as a range of variables, equations, with/without confidence intervals, and/or as a probability distribution (one can also choose to not discount anything). To assess the random nature of the trial, one can enter transition probabilities to transition from one state to the other states and simulate the trial via Markov models and monte carlo simulations or simulate the flow of the RCT processes via other simulation methods. The software and/or computational methods can be employed to model the randomized processes, such as via neural networks, Markov models, monte carlo simulations, stochastic processes, and/or via methods outlined in the (“Encyclopedia of Statistical Sciences” Samuel Kotz Campbell B. Read N. Balakrishnan Brani Vidakovic Norman L. Johnson); (“The Concise Encyclopedia of Statistics” https://doi.org/10.1007/978-0-387-32833-1_21); (“Simulation Modeling and Analysis (McGraw-Hill Series in Industrial Engineering and Management)” Averill Law); (“Stochastic Modeling: Analysis and Simulation (Dover Books on Mathematics)” B Nelson); (“Network Modeling, Simulation and Analysis in MATLAB: Theory and Practices” D Le); (“Introduction to the Modeling and Analysis of Complex Systems” H Sayama); (“Theory and Practical Exercises of System Dynamics: Modeling systems for analysis and optimization” (Modeling and Simulation 2020) by Juan Martín García and John Sterman), and/or any general modeling method. If one ran the initial program analysis on Base Models one could follow the assessment with a sensitivity analysis, based on assessing discrete variable and/or probabilistic variables as entered earlier and demonstrated in the additional paper included herein (in additional files section) or the references included in this application. Following the analysis, one can use the software to report the RCT CEA and/or optimization results automatically, or to provide graphical feedback to serve as a tool in the analysis of the results (the results type data that can come from the software is depicted in (Rafferty H, Rocha E, Gonzalez-Mego P, Ramos C L, El-Hagrassy M M, Gunduz M E, Uygur-Kucukseymen E, Zehry H, Chaudhari S S, Teixeira P E, Rosa G R, Zaninotto A L, Connor C, Eden U, Ramos-Estebanez C, Fregni F, Dipietro L, Wagner T. Cost-Effectiveness Analysis to Inform Randomized Controlled Trial Design in Chronic Pain Research: Methods for Guiding Decisions on the Addition of a Run-In Period. Princ Pract Clin Res. 2022 Jul. 3;8(2):31-42. doi: 10.21801/ppcrj.2022.82.5. Epub 2022 Aug. 22. PMID: 36561218; PMCID: PMC9769699)). Finally, the software module can make a recommendation of the RCT design that should be implemented through methods outlined above or when used with a patient or group of patients the software can be used to make a recommendation as to the most optimal therapy that can be used.

Additionally, while the system, software, and computational based methods is presented herein as a forward predicting system, it could also work in an inverse manner as a whole or in part (e.g., one would start with a desired Cost of a trial component and work the process backward). The software can work by requiring input from a user, be semi-automated, and/or fully automated. The analysis can also be completed or optimized using any machine learning and/or artificial intelligence such as those in (“Encyclopedia of Machine Learning and Data Mining” Claude Sammut and G. Webb); (“Deep Learning (Adaptive Computation and Machine Learning series)” Ian Goodfellow et. al,); (“Artificial Intelligence: A Modern Approach (4th Edition)” S. Russell and P. Norvig); (“Deep Learning (The MIT Press Essential Knowledge series)” J Kelleher).

The hardware system can be a single computer system with integrated software containing the above modules, multiple systems with individual software modules (or some combination), and/or via a host/client network approach (e.g., cloud-based computing). The hardware used can be a computer(s) and/or a mobile device(s) (e.g., phones, tablets). The hardware could include monitors, data entry devices (e.g., mouse, keyboard, touch screen monitor), computational processors, memory units, graphical processing units, and general computational components. The exemplary software, hardware, and/or computational based methods for determining an optimized design or cost-effective design for a clinical trial, such as a Randomized Controlled Trial (RCT) for evaluating medical therapies can be integrated into a motion analysis suite(s) as outlined herein (with or without other assessment methods and/or therapeutic options) to optimize “Value.” The system can also be deployed not as an RCT evaluator, but to determine the “cost effectiveness” and/or “value” of a treatment plan or plans to optimize therapy for a patient and/or group of patients.

Big Data Harmonization Examples: Tools for quality control, standardization of data acquisition, visualization, pre-processing, and analysis can be integrated into brain stimulation, neuromodulation, motion analysis suite(s), cost effective analysis software, diagnostics, prognostics, health care and/or combined elements such as for example with a motion analysis suite and methods that can aid care providers in motor symptom assessments, differential diagnosis, disease diagnosis, patient/disease classification, prediction of patient recovery, prediction of disease progression, prediction of treatment outcome, treatment tuning or optimization, prognosis, prediction of new symptoms development, prediction of hospitalization risk, prediction of needed level of assistance, diet optimization, and/or dosing of a treatment .. For example, in neuroimaging, quality control of acquired images is a long-standing problem. Traditionally, this is performed visually, but, in big data sets, large Volumes make this approach exceedingly expensive and impractical. Thus, methods for automatic quality control have can be used to correct for different imaging scanners (e.g., from different manufacturers, with different field strengths or hardware drifts). Another source of variability is data pre-processing techniques and pipelines. The motion analysis suite(s) are designed so that they can eliminate variability by providing a standard method of data assessment and furthermore allow access to open-access pre-processed datasets, such as that from the Preprocessed Connectome Project which systematically pre-process the data from the 1000 Functional Connectomes Project and International Neuroimaging Data-sharing Initiative (Biswal et al., Toward discovery science of human brain function, Proc Natl Acad Sci USA, 2010; Mennes et al., Making data sharing work: the FCP/INDI experience, Neuroimage, 2013) to facilitate use. The systems are also designed to be integrated with software like Combat (originally designed to remove batch effects in genomic data (Johnson et al., Adjusting batch effects in microarray expression data using empirical Bayes methods, Biostatistics, 2007) and later modified to manage DTI, cortical thickness measurements (Fortin et al., Harmonization of cortical thickness measurements across scanners and sites, Neuroimage, 2018) and functional connectivity matrices (Yu et al., Statistical harmonization corrects site effects in functional connectivity measurements from multi-site fMRI data, Hum Brain Mapp, 2018) to help researchers harmonize data from various types of study, regardless of whether they are analyzing newly collected data or retrospective data gathered with older standards). Tools for data visualization and/or interactive manipulation, such as Virtual Reality and/or Augmented Reality tools, can be integrated with the motion analysis suite(s) and/or other systems described herein.

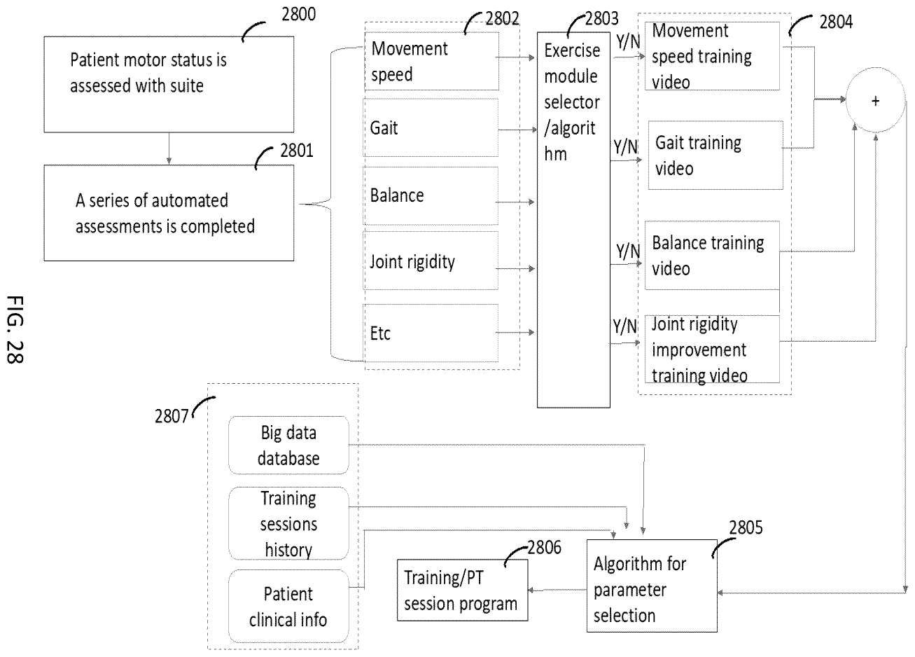

Combined Example and Precision Medicine Implementation: Differently from a traditional one-size-fits-all approach, Precision Medicine seeks to optimize patient care based on individual patient characteristics, such as information about a patient's genes, environment, movement characteristics, and lifestyle, to prevent, diagnose or treat disease. An example of a way to acquire large real-time multimodal data sets such as for use in personalized care in the movement disorder, pain, and rehabilitation spaces we have developed an Integrated Motion Analysis Suite (IMAS), which combines motion capture camera(s), inertial sensors (gyroscope/accelerometers), and force sensors to assess patient movement kinematics from joint(s) across the body and kinetics. For example, the IMAS can fill an unmet need in stroke rehabilitation, where the AHA Stroke Rehabilitation guidelines specifically call for the development of “computer-adapted assessments for personalized and tailored interventions”, “newer technologies such as . . . body-worn sensors”, and “better predictor models to identify responders and non-responders” (Winstein et al., Guidelines for Adult Stroke Rehabilitation and Recovery, A Guideline for Healthcare Professionals From the American Heart Association/American Stroke Association, 2016). However, our technology that can holistically aid clinicians in motor symptom assessments, patient classification, and prediction of recovery or response to treatment can be used not only in stroke (neurorehabilitation) but also in other motor disorders. The hardware system for movement kinematic and kinetic data capture is underpinned with an AI driven computational system with algorithms for data reduction, modeling, and predictions of clinical scales and prognostic potential for motor recovery (or response to treatment). The system is currently being used as part of a stroke study (“Clinicaltrials. gov. Clinical IMAS Optimization and Applicability in an Acute Stroke Setting. 2022.,”) and supporting other studies in the movement disorder (“Clinicaltrials. gov. Parkinson's Disease: Enhancing Physical Therapy With Brain Stimulation for Treating Postural Instability. 2022,”) and chronic pain (Clinicaltrials.gov, IMAS Optimization and Applicability in an Acute Stroke Setting, 2022) spaces. As for the big data component, the system has been designed so multiple systems can be networked together and multiple patients' kinematic/kinetic data, imaging, and clinical data can be longitudinally assessed and analyzed to develop a continually improving model of patient recovery (or as a method to personalize and optimize therapy delivery and predicting response to therapy-see below). The system is also designed to integrate with real-world data (e.g., EHR, payer databases) to further power the model. WE have also developed a new form of noninvasive brain stimulation, electrosonic stimulation (ESStim) (Wagner & Dipietro, Novel Methods of Transcranial Stimulation: Electrosonic Stimulation, Neuromodulation: Comprehensive Textbook of Principles, Technologies, and Therapies, 2018), and using it in a number of areas (e.g., diabetic neuropathic pain (Sukpornchairak et al., Non-Invasive Brain Stimulation For Diabetic Neuropathic Pain, American Academy of Neurology Annual Meeting, 2022), Carpal Tunnel Syndrome (CTS) pain (Clinicaltrials. gov, IMAS Optimization and Applicability in an Acute Stroke Setting, 2022), Parkinson's Disease (PD) (Wagner & Dipietro, Novel Methods of Transcranial Stimulation: Electrosonic Stimulation, Neuromodulation: Comprehensive Textbook of Principles, Technologies, and Therapies, 2018), and Opioid Use Disorder/Addiction (Clinicaltrials. gov, Optimization of NIBS for Treatment of Addiction, 2022)). The system(s) allows for assessment of stimulation efficacy through combined imaging data, biospecimen data, clinical data, kinematic data, and/or patient specific biophysical models of stimulation dose at the targeted brain sites to identify best responders to therapy (e.g., in PD, OUD, and Pain). The system(s) supports computational models to identify the best responders to therapy and/or as a means to personalize therapy based on the unique characteristics of the individual patients. The IMAS system, with its big data backbone, can be integrated with the ESStim system to further aid in personalizing patient stimulation dose in certain indications (e.g., PD, CTS pain). We can also integrate this system with our trial optimization tool based on health economics modeling (e.g., Cost Effective Analysis (CEA)) (Rafferty et al., Cost-Effectiveness Analysis to Inform Randomized Controlled Trial Design in Chronic Pain Research: Methods for Guiding Decisions on the Addition of a Run-In Period, Princ Pract Clin Res, 2022; Wagner et al., Noninvasive Brain Stimulation for Treating Chronic Pain and Addiction, Third Annual NIH HEAL Initiative Investigator Meeting, 2022). The software allows for a virtual trial design and predicting the trials cost effectiveness. Furthermore, the software can be implemented as a means to quantify data set values such as to quantitively support decision maker policy. Ultimately, the systems can be combined to allow for the use in a personalized treatment suite, based on a big data infrastructure, whereby the multimodal data sets (e.g., imaging, biophysical field-tissue interaction models, clinical, and biospecimen data) are coupled rapidly to personalize brain stimulation-based treatments in a diverse and expansive patient cohorts (see FIG. 27).

The imaging combination can be any type of imaging or the processed images, such as a connectome (i.e., DTI). As discussed, tuning and/or optimizing treatment (e.g., neurostimulation, physical therapy, drug therapy) and its effects are discussed in the various references incorporated herein. For example, the motion analysis system can be used as part of a deep brain stimulation (DBS) stimulation parameter tuning process whereby a patient undergoes an exam with a motion analysis system(s) as detailed herein to establish a baseline measure, such as for example quantifying a Parkinson's patient's baseline tremor, bradykinesia, rigidity, and/or postural instability characteristics. The patient could subsequently be provided brain stimulation via a DBS device and reassessed with the motion analysis system(s) to compare the patient's Parkinson's patient's during stimulation tremor, bradykinesia, rigidity, and/or postural instability characteristics to the baseline characteristics. Furthermore, the practitioner could vary the DBS stimulation parameters (e.g., voltage, current, pulse frequency, pulse width, pulse shape, electrode lead, polarity) and assess the change in the data from the motion analysis system(s) to determine the stimulation parameters which improve the patient's symptoms. One could also use the process to selectively tune the patient's response, such as for example maximally improving a patient's postural instability, while potentially having less effect on other symptoms (e.g., tremor) such as for example in a patient that had a history of falls. As another example, one could use the system combined patient clinical information, such as presence of comorbidities, to predict the risk for hospitalization, fall, fracture risk, and/or likelihood of recovery. This set of data can also be used to predict whether and when the patient will need assistance for an independent life and what type of assistance (e.g., caregiver vs aids). This information can be used to create a tool for financial planning (as can the software component to assess value and cost effectiveness of treatment plans). As another example, certain foods are known to interfere with levodopa therapy and decrease its effects on motor symptoms. MAS data can be used to measure the effects of different foods on motor performance as a function of levodopa time assumption so that a patient can optimize their diet to avoid interference with levodopa assumption. As another example, a set of network connected motion analysis suite(s) can be used with multiple patients, either in discrete or ongoing evaluations, following the exemplified tuning process, and a central computation system could evaluate this discrete or expanding data set via the statistical/AI based methods described herein and/or the incorporated references to tune the stimulation patterns. A big data approach and/or an adaptive model approach can be implemented where ongoing evaluations from large numbers of patients can be continually implemented to continually improve the stimulation tuning. Such a method can be integrated with other patient data sets to further optimize the stimulation (e.g., EEG, MRI, SPECT brain scan, DaT scan., EKG, patient history, behavioral assessments, clinical exam data, biospecimens, cognitive assessments). Such as for example, numerous motion analysis systems can be connected via the internet but deployed to multiple clinical sites where stroke patients are assessed and provided physical/rehabilitation therapy. The connected systems could initially assess a patient's baseline information and be used to collect additional data, such as patient imaging data and/or clinical assessments, which can be uploaded to a central system which develops mathematical models, such as a linear model or correlation models, described the relationship between the patient's motor exam values gathered with the motion analysis systems and the clinical assessments and/or imaging data. The uploaded data or generated models can further be used to classify patients (and/or potentially identify patients that would best respond to specific physical therapy/rehabilitation regimens). As more patients are evaluated and/or the patient(s) or patients begin and/or continue to undergo treatment, further data can be uploaded to the central system and the classification and/or therapy tuning can further be improved and optimized. Furthermore, as the classification and/or treatment can be influenced by this motion analysis system(s) based exams, such as by using feedback, one can continuously optimize the classification and/or treatment process by providing the feedback to those conducting the motion analysis system-based exams. The multiple motion analysis systems can be connected to a central computation system, or the connected multiple motion analysis systems can work in parallel to complete the computational processes. As demonstrated, numerous computational methods can be used to complete such a process, and the methods described herein should be considered exemplary as one skilled in the art would note other such methods can be employed.

In another embodiment the motion analysis suite is used to assess patient motor abilities and/or this data is matched with specific physical therapy exercises that are provided to the patient in the form of videos (such as for example on the screen of the motion analysis suite and/or on a separate viewing device (e.g., external monitor, watch screen, tablet, screen, phone screen, television, and/or projection system)) and/or other instructions (e.g., verbal, written, and/or graphical instructions provided directly on the motion analysis suite screen and/or an alternate source(s) (e.g., headphones, speaker, separate viewing device)). For example, if the suite and its algorithms and/or a diagnosis from a care provider find that the patient movement is bradykinetic a video provided to the patient shows motor exercises aimed at improving movement speed; if the suite and its algorithms and/or a diagnosis from a care provider find that a patient joint is rigid the video provided to the patient shows motor exercises aimed at improving rigidity. One or more videos can be provided to the patient. The videos can be combined to generate an exercise program for a session. The videos can be selected and/or combined in multiple ways, including manually, using a look-up table, and/or algorithms of different complexity. For example, an algorithm can select all the videos that should be used for the session and another algorithm can refine the choice and select the dosage of each exercise (e.g., determine the optimal length or repetitions for each exercise under constraints set by the user (e.g., prioritizing some exercises/physical therapy goals or keeping the session length within a certain time frame)). In this embodiment, the motion analysis suite can be used to periodically assess the patient progress and its data and algorithms can be used to devise more (or less) challenging physical therapy exercises based on patient achievements. An example flowchart of this embodiment is shown in FIG. 28, where the assessment is performed by the suite 2800 that uses its sensors and algorithms to automatically (or semiautomatically) assess 2801 whether there are impairments in a series of specific motor abilities (2802; examples include but are not limited to gait and posture). Each impairment is associated to a video for training that specific ability. A video or multiple videos 2804 are selected by a selector module 2803 in order to build a program of exercises for a session 2806. An AI algorithm can be used to further refine the exercise program for the session 2805, e.g., by choosing the number of repetitions/dosage of each exercise. To accomplish this step, the algorithm can incorporate several other data from the patient under examination or from patients with similar characteristics e.g., in terms of motor abilities, impairments, comorbidities, and/or age. This data 2807 can include, but is not limited to, the patient clinical information or data from other patients for example stored in a big data database.

In another embodiment the motion analysis suite is used to assess patient motor abilities and/or its data are used to match the patient with specific aids, orthoses, and/or footwear. For example, if the suite and its algorithms and/or a diagnosis from another care provider find that the patient suffers from freezing of gait the suite data and its analysis algorithms can be used to match the patient with a cane or walker for assisting with movement that cue the steps by projecting a laser line. In another example if the suite and its algorithms and/or a diagnosis from another care provider find that the patient has an impaired gait the suite data and its analysis algorithms can be used to match the patient with specific footwear or orthosis. The matching can be done in several ways, for example showing a list of options to the patient, reassessing the patient motor abilities when they use the selected option, and comparing the data taken from the patient with and without the walking aid and/or orthosis and/or footwear and/or wheelchair; or an algorithm can be used to select the best option by analyzing data from a big data database containing data of patients with motor abilities similar to those of the patient under examination.

In another embodiment the motion analysis suite is used in conjunction with a videogame system where the videogame is specifically designed to train/exercise movements that are impaired by the disease. For example, for a Parkinson's patient that has problems with procedural motor learning the videogame can show on a screen the patient a series of movements to be performed in a certain order. The suite can be used to record the movement performed by the patient. An algorithm can calculate the distance between the movement kinematics of the movement performed by the patient and a pre-recorded ideal movement performed by a normal subject and calculate a distance score.

In another embodiment the system(s) discussed herein and/or its algorithm(s) can be integrated with a model or use a model such as a Natural Language Processing model and/or with a Large Language Model such as to facilitate communication and/or automate processes taking place with the system(s). Such as for example the system could be trained to provide optimal instruction for a patient to perform an exercise for maximum therapeutic effect (e.g., guide a Parkinson's patient in walking exercises based on the motion analysis suite information and/or coupled with the patient feedback). In another embodiment the system(s) discussed herein and/or its algorithm(s) can be integrated with a model for Generative Artificial Intelligence (AI) such as to facilitate communication (e.g., AI trained on items such as text, code, images, music, and/or video and/or AI used to provide outputs such as text, code, images, music, and/or video), provide a provide visual communications or figures such as for aiding in explaining activities, provide molecular data information (e.g., AI trained on molecular data such as part of biospecimen(s) and/or AI used to provide outputs of molecular data such as part of biospecimen (s)), provide movement information whereby the generative AI is trained on patient movements to generate output trajectories of new movements such as could be used for therapy (e.g., physical therapy, occupational therapy, sports therapy, and/or to optimize athletic training), provide verbal and/or sound information, and/or automate processes taking place with the system(s). For example, the motion analysis system can be trained on patient movements to identify specific disease patterns and further identify specific therapeutic movements via physical therapy that could benefit a patient or group of patients. As another example, the system could be trained on skilled athletes preforming a task and used to train less skilled athletes such as for example in a virtual coaching manner. Generative Planning can be used to generate a sequence of actions to reach a certain goal (such as a therapy routine to improve recovery from a stroke and/or to minimize fall risk in a class of patients). Generative AI can also be used for Data Privacy, Security and Governance. For example, the system(s) and/or its algorithms can use Generative Adversarial Networks (GANs), Variational Autoencoders (VAEs), Autoregressive Models, Recurrent Neural Networks (RNNs), Transformer-based Models, Reinforcement Learning for Generative Tasks, and/or Flow-Based Models.

In another embodiment the motion analysis suite is used to assess patient motor learning abilities. In this embodiment, the patient is asked to perform tasks with the upper or lower limb that require motor learning. The suite and its algorithms for analysis of movement kinematics and kinetics can be used to assess to which degree motor learning is impaired. For example, this can be done via comparison of the patient data with normative data taken from age-matched unimpaired subjects. This output can be used for a variety of applications, such as matching the patient with appropriate training exercises aimed at improving motor learning or aids to improve independence.

The disclosure may be embodied in other specific forms without departing from the spirit or essential characteristics thereof. The foregoing embodiments are therefore to be considered in all respects illustrative rather than limiting on the disclosure described herein. It should also be noted herein the term motion analysis system and motion analysis suite is used interchangeably for different embodiments herein and should not be taken as a limiting term.

### EXAMPLES

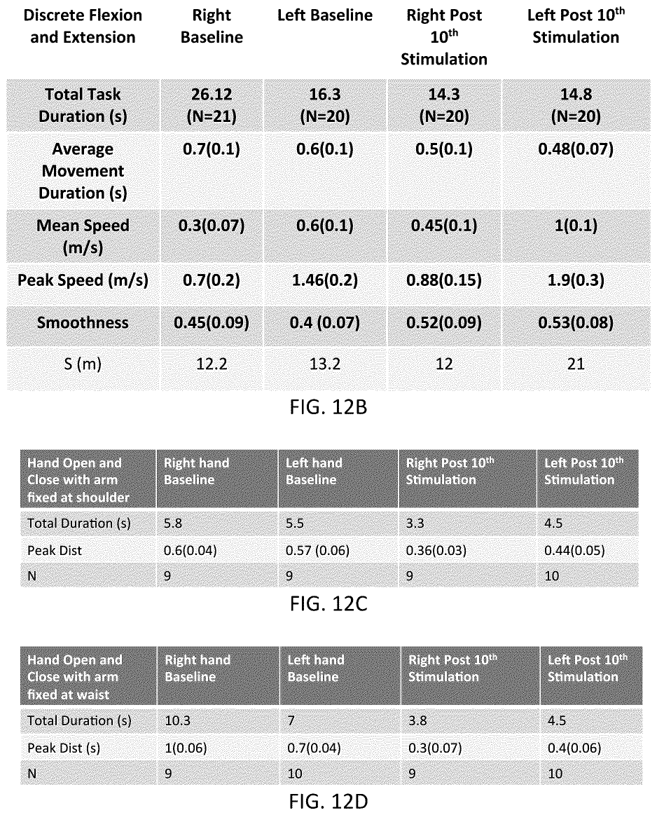

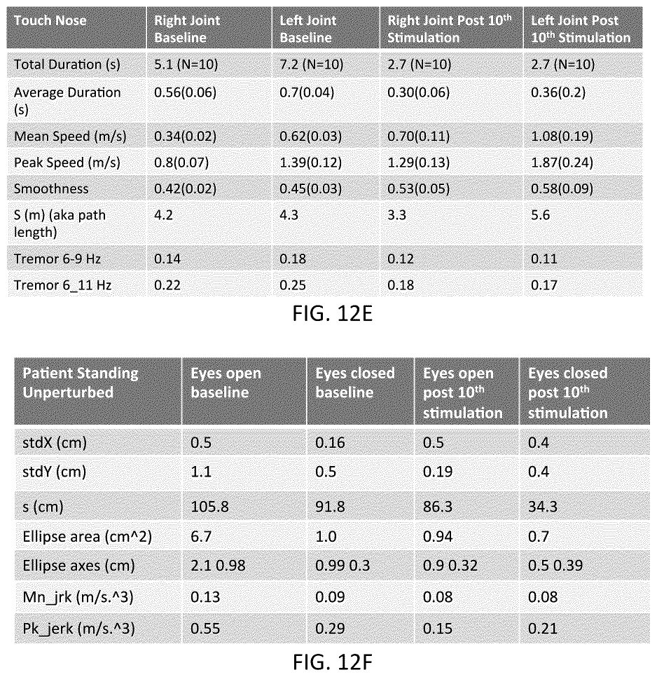

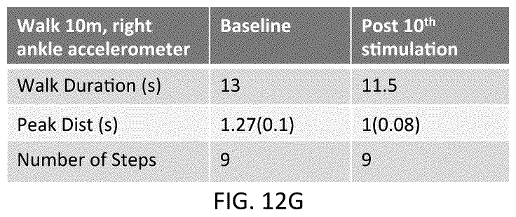

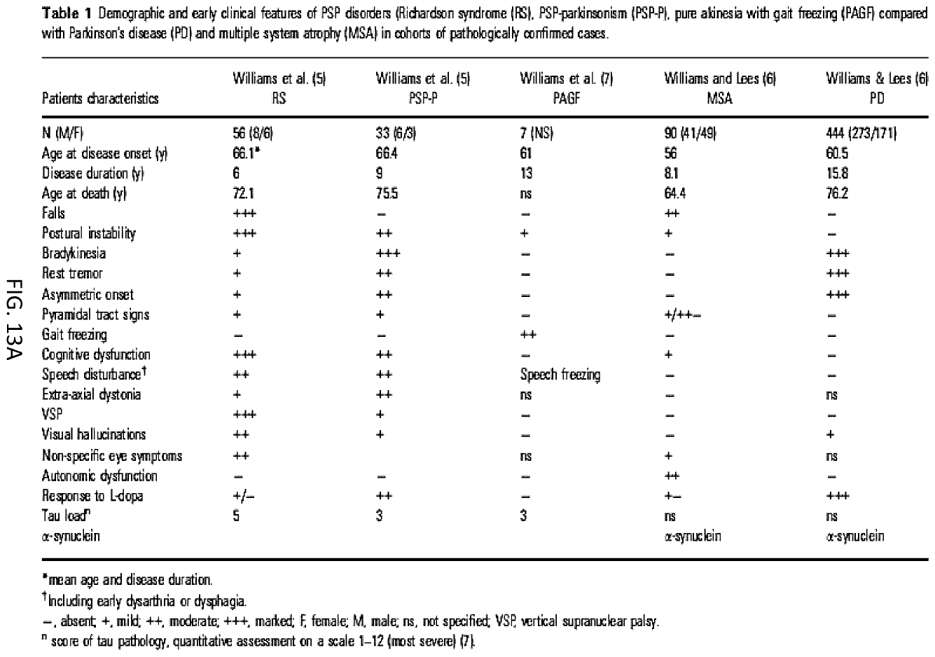

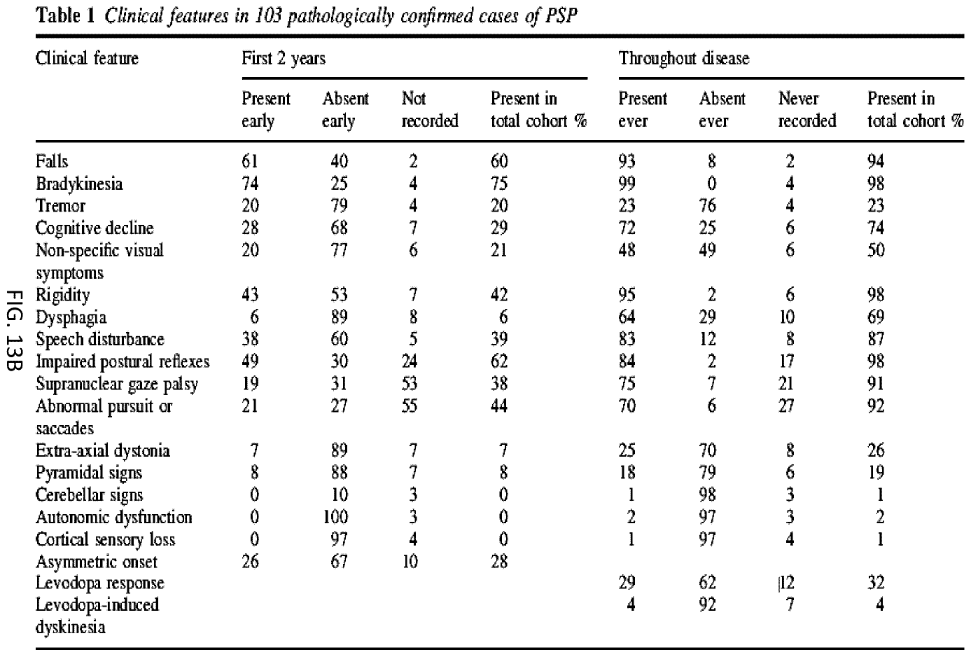

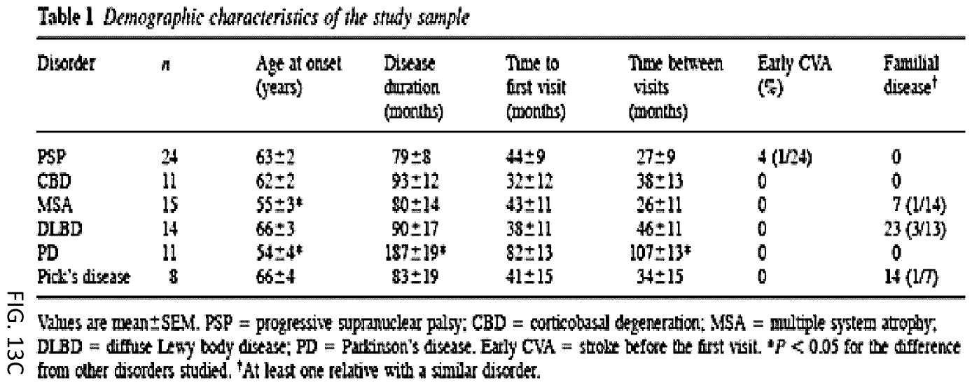

We implemented a stimulation system that provided electro-sonic stimulation (ESStim) to a Parkinson's disease patient's primary motor cortex 5 days a week for twenty minutes a day over 2 weeks (10 days total). We had the patient perform a bradykinesia test where the patient was asked to perform 10 arm flexion-extension movements as fast as possible while the patient was analyzed with the motion analysis system (the test and analysis are described above; where, baseline (i.e., before any stimulation was provided) measurements from this patient are provided in FIG. 6 and used to specifically describe the implementation of the analysis used for this task as described above). We conducted these tests at baseline (i.e., before any stimulation was provided), throughout the course of stimulation, and for weeks following stimulation. The following metrics were determined: movement mean speed (mean value of speed), movement peak speed (peak value of speed), movement duration: difference between offset of movement and onset of movement, movement smoothness (mean speed/peak speed). Also calculated is the path length of the trajectory of the wrist joint (distance traveled in 3D space). Results for this task, from the 10^{th} day stimulation and the baseline of the patient are provided in FIG. 12A.

We had the patient perform a bradykinesia test where the patient was asked to perform 10 arm flexion-extension movements. After each flexion or extension movement, the subject is asked to stop. The movements were performed as fast as possible (the test and analysis are described above, and FIG. 7 was based on the baseline information for this patient). Results for this task, from the 10^{th} day stimulation and the baseline of the patient are provided in FIG. 12B.

We had the patient perform a task where they were asked to perform 10 hand opening and closing movements, as fast as possible, while the hand was positioned at the shoulder (the test and analysis are described above, and FIG. 8 was based on the baseline information for this patient). Results for this task, from the 10^{th} day stimulation and the baseline of the patient are provided in FIG. 12C. In FIG. 12D, we provide a similar set of results for the patient where the task was performed with the hand positioned at the waist.

We had the patient perform a task where they were asked to combine the movements (flexion followed by hand opening/closing followed by extension followed by hand opening/closing) 10 times as fast as possible. Analysis based on the combined analysis of the individual motions was completed, and similar improvements were shown. For example, the patient took 26.6 and 23.6 seconds to perform the tasks at baseline (for the right and left joints respectively) and 23.6 and 20.6 seconds for the tasks after the 10^{th} stimulation session.

We had the patient perform a task where they were asked to touch their nose with their index finger, as completely as possible, 5 times (the test and analysis are described above, and FIG. 9 was based on the baseline information for this patient). The following metrics are the final output for this test: total duration of test; number of movements actually performed; movement mean speed (mean and standard deviation across all movements); movement peak speed (mean and standard deviation across all movements); movement duration (mean and standard deviation across all movements); movement smoothness (mean and standard deviation across all movements); path length; tremor in the range 6-9 Hz; tremor in the range 6-11 Hz. Results for this task, from the 10^{th} day stimulation and the baseline of the patient are provided in FIG. 12E.

We had the patient perform a task where the patient's posture and/or balance characteristics are assessed. The patient was asked to stand on a force plate while multiple conditions are assessed: eyes open and eyes closed. During measurements with eyes open or closed, the subject was simply asked to stand on a force plate. The test and analysis are described above, and FIG. 10 was based on the baseline information for this patient. Results for this task, from the 10^{th} day stimulation and the baseline of the patient are provided in FIG. 12F.

We had the patient perform a task for assessing gait and/or posture, where the patient was asked to walk 10 meters. The test and analysis are described above, and FIGS. 11A and 11B was based on baseline information for this patient. Results for this task, for the right ankle accelerometer and associated measures, from the 10^{th} day stimulation and the baseline of the patient are provided in FIG. 12G.

In other implementations, such as for example where stimulation is given to other patients who are less responsive to stimulation, for patients given less stimulation (i.e., a lower dose of stimulation), or for less effective types of stimulation the motion analysis system we describe herein can still be effective in demonstrating the effects of stimulation. Similarly in other implementations, such as for example where stimulation is given to other patients who are more responsive to stimulation, for patients given more stimulation (i.e., a larger dose of stimulation), or for more effective types of stimulation the motion analysis system we describe herein can still be effective in demonstrating the effects of stimulation.

For example, in another Parkinson's Disease patient, receiving the same stimulation protocol, and assessed with the bradykinesia test where the patient was asked to perform 10 arm flexion-extension movements as fast as possible while the patient was analyzed with the motion analysis system. For their right arm they demonstrated a baseline total time of task of 11.58 seconds, and average movement duration of 0.568 seconds, a mean speed of movement of 0.558 m/s, and a peak speed of 1.201 m/s. Following the 10^{th} simulation they demonstrated a total time of task of 13.3 seconds, and average movement duration of 0.6633 seconds, a mean speed of movement of 0.7199 m/s, and a peak speed of 1.52 m/s. In this patient they took longer to perform the total task, but were faster on all of the movements, and moved through a greater range of motion (i.e., the path length of the total movement can be calculated from the image capture device information). In this same patient we the patient perform a bradykinesia test where the patient was asked to perform 10 arm flexion-extension movements. After each flexion or extension movement, the subject is asked to stop. The movements were performed as fast as possible. For their right arm they demonstrated a baseline total time of task of 24.125 seconds, and average movement duration of 0.724 seconds, a mean speed of movement of 0.525 m/s, and a peak speed of 1.20 m/s. Following the 10^{th} simulation they demonstrated a total time of task of 18 seconds, and average movement duration of 0.73 seconds, a mean speed of movement of 0.582 m/s, and a peak speed of 1.27 m/s. We had the patient perform a task where they were asked to combine movements (flexion followed by hand opening/closing followed by extension followed by hand opening/closing) 10 times as fast as possible. For their right arm, the patient had a baseline total task time of 32.03 seconds and a total task time of 26.5 seconds following the 10^{th} day of stimulation.

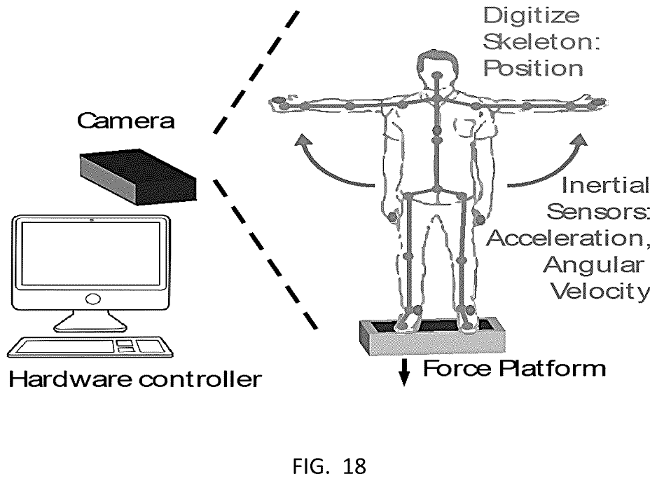

In another example of the implementation and application of a motion analysis system, we used a force plate (Wii board; ˜100 Hz sampling rate), two wearable accelerometers and gyroscopes (Inertial Measurement Unit (IMU) sensors; ˜64 Hz sampling rate), a portable camera-based system (˜30 Hz sampling rate) and a remote controller to evaluate patients with Parkinson's Disease (PD) (see FIG. 18). For the purpose of this example, we will refer to all of the above sensors as “motion analysis system.” The camera system included an embedded infrared sensor for measuring depth (such as can be found in Andersen, M. R., et al., Kinect Depth Sensor Evaluation for Computer Vision Applications, in Technical report ECE-TR-6. 2012, Department of Engineering—Electrical and Computer Engineering, Aarhus University), i.e., recording in 3D, and commercial software for segmenting the human body from background, modeling the body as a 20-joint skeleton (hip center, spine, shoulder center, head, left and right shoulders, elbows, wrists, hands, hips, knees, feet, and ankle joints), and tracking 3D positions of the 20 joints (Shotton, J., et al., Real-Time Human Pose Recognition in Parts from Single Depth Images. Communications of the ACM, 2013: p. 116-124; Han, J., et al., Enhanced computer vision with microsoft kinect sensor: A review. IEEE Trans. Cybern., 2013 43(5): p. 1318-1334) (indicated as green circles in FIG. 18). In this example twenty-nine patients with PD were evaluated. First, subjects were evaluated with UPDRS by a clinician/neurologist. As part of UPDRS testing, UPDRS III motor scores were collected (14 subcomponents of the UPDRS scale, i.e., UPDRS Q18-31). The UPDRS III total score was obtained as the sum of the UPDRS Q18-31 scores (Goetz, C. G., et al., PMID: 7544438). Evaluations were performed during ON periods and focused on subjects' most affected side (left or right), as self-reported. Then, subjects were asked to perform a series of motor tasks designed to assess bradykinesia, ability to perform complex movements, tremor, postural instability, and gait, while their movements were tracked using our motion analysis system. These evaluations focused on subjects most affected side (left or right), as self-reported.

Motor tasks used for this analysis were as follows: 1) continuous elbow flexion/extension movements: subject was instructed to move as fast as possible, keeping the wrist stable, palm up, beginning at level of waist/hip, going up to shoulder without touching it or overextending, and keeping the elbow stable but not pressed to the side (10 repetitions); 2) discrete elbow flexion/extension movements: similar to 1), but stopping for 2 seconds at the end of each movement without letting the hand flop, and going as fast as possible in between; 3) hand opening/closing at shoulder level: subject was instructed to fully open and close their hand fully in a fist (not clenching hard) as fast as possible, keeping the hand at the shoulder level (10 repetitions) starting with the hand open; 4) hand opening/closing at hips/waist level: similar to the test described in 3); 5) complex motor sequence: subject was instructed to perform the hand opening/closing movements at the waist/hip and shoulder, and to perform the flexion/extension movements as fast as possible in between with the hands open (10 repetitions); 6) hand-to-nose: keeping the arm/elbow at shoulder level, subject was asked to bring their hand (horizontal, palm down) almost to their nose without touching it and to extend it all the way to the side again, beginning with the arm outstretched and moving at their natural pace (10 repetitions); 7) hand resting on table: subject was asked to rest their hand and forearm on a table, with the arm relaxed, for 30 seconds while fixating the evaluator's index finger swinging back and forth; 8) hand resting in front of face: with arm/elbow at shoulder level, subject was asked to take their hand close to their nose and keep the hand there for 15 seconds while fixating the evaluator's index finger swinging back and forth; 9) balance test: with feet positioned in the middle of each side of a Wii board (i.e., feet about shoulder width apart) subject was asked to maintain an upright position for 30 seconds; test was performed twice, once with eyes open (while fixating a pre-defined landmark) and once with eyes closed; 10) walking test: subject was asked to walk for 10 meters at their usual pace (4 repetitions). Tests 1-8 were performed in seated position. Subjects wore hospital socks throughout tests 9-10. Subjects' motor performances during all tests were monitored by motion analysis system.

All patients were evaluated twice, over two separate days about one week apart. During the two evaluations we collected motion analysis system and UPDRS III data.

The IMUs were attached to the subject's body with Velcro straps, using anatomical landmarks for guiding positioning. Specifically, they were placed as follows: for tests 1-8, an IMU was placed on the top side of the patient's index finger; for the balance tests, an IMU was positioned on the subject's back, at the level of L5, near the body's center of mass; for the walking tests, patient's movement was tracked with two IMUs, one on L5 and one on the right ankle using the lateral malleolus as landmark for the first 2 repetitions; for the last 2 repetitions, each ankle (right and left malleoli) was tracked with a separate IMU. A remote controller allowed the experimenter to mark recordings; the marker signal was set whenever an event occurred (e.g., beginning or end of each motor task) and was null otherwise.

All the motor tests were tracked with the camera system, except for the walking tests. For the balance test, patients were asked to stand on the force plate. For the whole duration of the experiments, camera, force plate, and subject's chair were kept in fixed positions to minimize set-up times between sessions and prevent errors due to equipment re-positioning, and also to maintain consistency between participants.

Custom C# routines were written to synchronize the recordings from all the sensors and the remote controller. While C# was used in this example, any computational languages such as python, visual basic, Matlab, assembly, java, mobile system languages such as for developing apps for android or apple mobile operating systems, etc. can be implemented.

Quantitative metrics were extracted from the motion analysis system data recorded during the above motor tests. For all tasks we calculated the total task duration, as the difference between the end and the start of the task, which were automatically extracted by the marker signal as, respectively, the last and the first time the marker signal became positive. Additionally, the following metrics were calculated:
To assess bradykinesia from tasks 1-2 (proximal motor system), wrist movements speed profiles s were calculated from the first order derivative of the 3D wrist trajectories (X, Y, Z wrist trajectory components) smoothed with a 10 Hz low-pass FIR filter) similar to (Vaisman, L. et al PMID: 23232435; Dipietro, L. et al. PMID: 22186963) then, speed profiles s were segmented to extract individual movements; to this end, the time instants corresponding to peaks of-s were calculated and the portions of data between the times corresponding to two successive peaks were labeled as individual movements (i.e., start and end of each individual movement). For each individual movement we computed movement mean speed, max speed, duration, and smoothness (calculated as the ratio between mean speed and max speed as in (Dipietro, L. et al. PMID: 22186963 (Dipietro et al., Learning, not adaptation, characterizes stroke motor recovery: evidence from kinematic changes induced by robot-assisted therapy in trained and untrained task in the same workspace, IEEE Trans Neural Syst Rehabil Eng, 2012)). Similar metrics were used to assess patient ability to complete the movements recorded during task 6.

To assess bradykinesia from tasks 3-4 (distal motor system), angular velocity signals from the gyroscope (Xᵣₒₜ, Yᵣₒₜ, Zᵣₒₜ) were filtered with a 4^{th} order low-pass Butterworth filter (5 Hz cut-off). For these tasks, metrics included movement time (calculated as total time divided by the number of movements) and inter-peak interval (interval between consecutive times when the hand was fully open, as marked by positive peaks in the angular velocity component Xᵣₒₜ).

For all the above tests, mean and standard deviation of all the metrics across all the repetitions were calculated for each subject.

Analysis of task 5 focused on total time to complete the task. To assess resting tremor, the fractional power in the 3-6 Hz band was calculated from the accelerometer amplitude (computed from the 3D acceleration components) from test 7; specifically, we calculated the ratio of mean power in the 3-6 Hz band and mean power through the measured band (0-32Hz), where power was evaluated with multitaper spectral analysis (Chen, H et al. PMID: 16357337; Gonzalez-Usigli, H. A. and A. Espay, Overview of Movement and Cerebellar Disorders, in Merck Manuals Professional Edition. 2013; Behtash B. and Brown E. N., PMID: 24759284; Teräväinen, H. and Calne, D. B. PMID: 7373323) (other methods were also explored, i.e., ratio of the power in the 3-6 Hz frequency band and total power; both metrics were also calculated using FFT); a similar method was used to assess postural tremor (5-8 Hz) from the accelerometer recordings from test 8.

To assess balance from test 9, the length of the path traveled by the subjects' body center of pressure (CoP) as measured by the force board where the subject was standing was calculated similar to (Schmit J. M. et al. PMID: 16047175). In order to further characterize postural sway, standard deviation of CoP components and the parameters of an ellipse fitting CoP oscillations (axes length and area) were calculated using a fitting method similar to the one described in (Dipietro, L. et al. PMID: 22186963). In order to measure smoothness of postural sway (Mancini, M. et al. PMID: 21641263), the mean and peak values of jerk (first-order derivative of acceleration) amplitude was calculated from the acceleration measured by the IMU placed on L5 along the antero-posterior and medio-lateral directions similar to (Mancini, M. et al. PMID: 21641263; Mancini, M. et al. PMID: 22913719). Separate values for the eyes open and eyes closed tests were calculated.

To evaluate walking, besides the total task duration (see above), the following metrics were calculated from the IMU recordings after signals were filtered (4^{th} order Butterworth low-pass filter, 5 Hz cutoff). For walks 1-2, movement smoothness was calculated as normalized jerk (mean jerk magnitude divided by mean speed (Rohrer, B. et al. PMID: 12223584) where jerk amplitude was calculated from the first order derivatives of the filtered components of the signals recorded from the accelerometer mounted on L5, smoothed with a 4^{th} order low-pass (5 Hz cutoff) Butterworth filter. Averages of each metrics across the trials were reported. For walks 3-4, we localized the peaks of the Zᵣₒₜ gyroscope signals (the angular velocity component where movements were most evident) to assess when strides occurred; then, we calculated the distance between successive peaks (stride duration). Thus, averages of each metrics across trials, namely number of strides (from number of peaks), stride duration and its standard deviation, were calculated.

Custom MATLAB routines were written to automatically extract the metrics from the motion analysis system recordings. While Matlab was used in this example, any computational languages such as python, visual basic, C, assembly, java, mobile system languages such as for developing apps for android or apple mobile operating systems, etc. can be implemented.

Pearson correlation was calculated between each motion analysis system metric and UPDRS III scores. The level of significance was set to 0.05.

Principal Component Analysis (PCA) (Jolliffe, I. T., Principal Component Analysis. Springer Series in Statistics, ed. Springer. 2002) was used to examine the correlation structure in the UPDRS III and motion analysis system metrics and to estimate the effective dimensionality of both data sets. Each measure in each data set was standardized by removing its mean and dividing by its standard deviation and separate PCAs were conducted for the set of UPDRS III and motion analysis system measures, separately and together. The motion analysis system metrics data set consisted of 65 measures per subject and evaluation day, including mean and standard deviation across repetitions for tests that entailed multiple repetitions.

A sparse linear model using LASSO optimization Tibshirani, R., Regression Shrinkage and Selection via the Lasso. Journal of the Royal Statistical Society., 1996. 58(1): p. 267-288) was used to assess the ability to predict total UPDRS III from the motion analysis system data and identify sparse set of predictors from the motion analysis system dataset. Using these predictors, we estimated the complete leave-one-out cross validation prediction quality, fitting a model using these predictors to complete set of patients with one removed and using that model fit to predict the UPDRS III value for the remaining patient (Geisser, S., Predictive Inference. 1993, New York, NY: Chapman and Hall).

These analyses were performed using custom routines written in MATLAB (The Mathworks, Natick, MA).

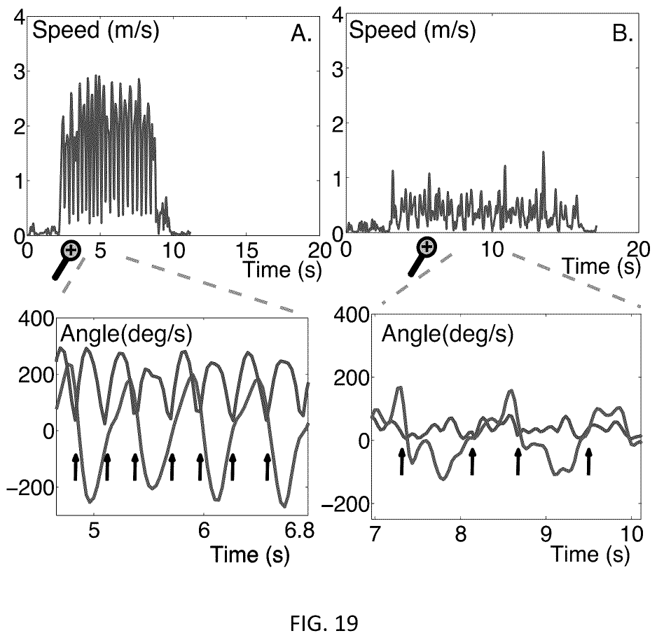

For the example study reported here, the average age of the 29 patients was 64.83 (SD 11.29) years; 6 patients were females and 23 males. UPDRS III total scores ranged from 5 to 29 (average 14.98(7.10). Subject's most affected side was the right side for most subjects (N=20). FIG. 19 shows typical wrist speed profiles of two PD patients with different UPDRS III scores, as derived from the camera system recordings during test 1. The patient with the UPDRS III score of 21 (i.e., greater motor impairment) (top right panel) was slower than the patient with the UPDRS III of 9 (left panel), with mean speed=0.37 m/s (0.06 ) vs. 1.6 m/s (0.21 ), standard deviation in parentheses; max speed=0.81 m/s (0.23 ) vs. 2.5 m/s (0.33 ), and movement duration=0.61 s (0.12) vs. 0.30 s (0.06); their movement smoothness was also worse, 0.48 (0.09) vs. 0.64 (0.07). Similar results were found for test 2 (bottom panels). Similarly, for the hand opening/closing tests, compared to the patient with the UPDRS III of 9, the patient with a score of 21 moved more slowly (average time was 0.65 s/movement vs 0.47 s/movement for test 3 and 1.08 s/movement vs 0.46 s/movement for test 4), took longer for completing the complex, multi-joint motor tasks of test 5 (26.7 s vs 20.06 s respectively), and performed the hand-to-nose movements less easily (movement quality as measured by movement smoothness was worse, namely 0.41 vs 0.53) and more slowly (average mean speed was 0.34 m/s vs 0.89 m/s and average max speed was 0.81 m/s vs 1.69 m/s, although it should be noted that this task did not specifically aim at testing bradykinesia).

The patient with the UPDRS III of 21 showed a more prominent resting tremor, with 74.5% greater power than the patient with the UPDRS III of 9 (1.54 vs. 0.88). Slightly higher values were found for kinetic tremor and limited differences for postural tremor in these two patients.

FIG. 20 shows CoP oscillations for the patient with UPDRS III of 21 (right panel) and the patient with UPDRS III of 9 (left panel). The patient with higher score showed poorer postural control and less smooth movements than the patient with lower UPDRS III when the test was conducted with eyes open, with path length=142.7 cm vs. 23.57 cm; the difference in postural control ability was also reflected in the jerk metrics (mean jerk=0.14 m/s³ and max jerk=0.55 m/s³ vs mean jerk=0.03 m/s³ and max jerk=0.13 m/s³, respectively for the patient with UPDRS III of 21 vs 9). Similar results were found for the test when conducted with eyes closed (path length=91.87 cm vs 23.58 cm; mean jerk=0.09 m/s³ and max jerk=0.29 m/s³ vs 0.03 m/s³ and 0.10 m/s³, respectively for the patient with UPDRS III of 21 vs 9).

Using methods similar to above, it was shown that patient with the UPDRS III of 21 showed greater walking impairment than the patient with the UPDRS III of 9, with average total walking times of 13 s vs. 7.25 s, most affected leg average stride times (Tresilian, J., Sensorimotor Control and Learning: An Introduction to the Behavioral Neuroscience of Action. 2012: Palgrave Macmillan) of 1.27 s vs. 1.04 s, stride lengths of 1.0 m vs. 1.67 m, and stride velocity of 0.78 m/s vs. 1.6 m/s.

Across the patient set (n=29) and both evaluations with the motion analysis system, total UPDRS III scores significantly correlated with each of the separate motion analysis system speed measures for test 1 with values of −0.32, −0.30, 0.59, and −0.28 respectively for mean speed, max speed, movement duration, and movement smoothness. Similar results were found for test 2 (−0.34, −0.30, 0.50 (p<0.05), respectively for mean speed, max speed, and movement duration)), except for movement smoothness (−0.15 (p=0.25)); for hand opening/closing test at hips/waist level (0.29 for movement speed as measured by interpeak distance); for complex movement task of test 5 (0.28 for movement duration); and for the hand-to-nose task with values of −0.47, −0.48, and 0.34 for mean speed, max speed and movement duration, respectively. Interestingly, for the hand-to-nose movements standard deviation of mean speed and peak speed across repetitions significantly correlated with the total UPDRS III scores (−0.43 and −0.32 respectively), possibly reflecting movement speed-accuracy tradeoff which is preserved in PD patients (Fernandez, L. et al. PMID: 30405521). Additionally, most measures of movement speed were also significantly correlated with the UPDRS III body bradykinesia subcomponent (e.g., movement duration for test 1 (0.52) and test 2 (0.43), total movement duration in test 3 (0.32) and test 4 (0.39), and total movement duration in test 5 (0.27)).

Among the tremor metrics, only the resting tremor motion analysis system metrics significantly correlated with the UPDRS III total (all metrics for test 7 were significantly correlated with total UPDRS III scores with correlation values ranging from 0.46 to 0.50), but both resting and postural motion analysis system tremor metrics were significantly correlated with the UPDRS III tremor subcomponent with correlation values ranging from 0.42 to 0.52. Among the balance metrics, the peak jerk was significantly correlated with the total UPDRS III scores (0.3 for the open eyes test); however, similar to (Mancini, M. et al PMID: 21641263), the CoP path length did not correlate significantly with UPDRS III total scores. All the gait metrics correlated with the total UPDRS III, except for stride times (e.g., 0.57 for both task duration and stride count). These results suggest that the different motion analysis system metrics are able to capture different levels of motor impairment as clinically measured by the total UPDRS III score, but the ability of each separate motion analysis system metric to predict the total UPDRS III score is modest as shown by the results of the correlation analysis.

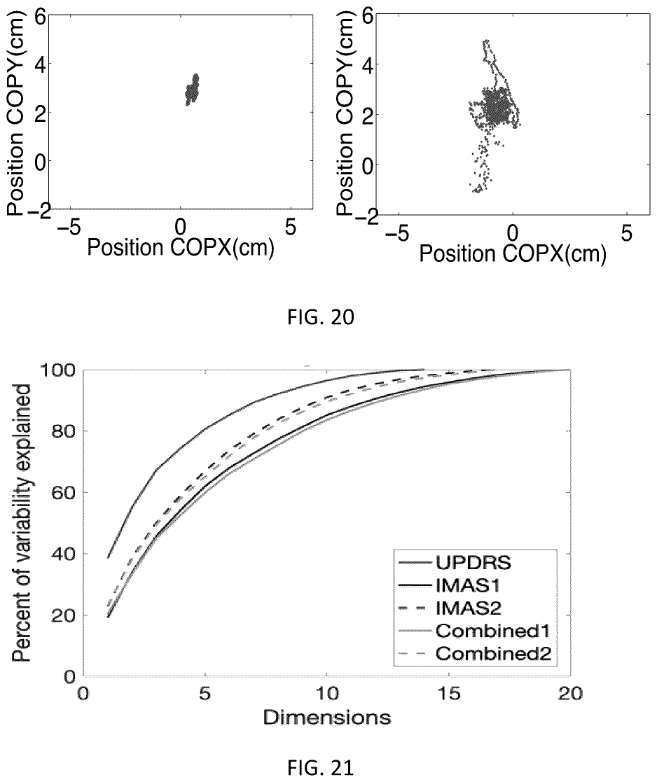

FIG. 21 shows the PCA results. Each line represents the percentage of total variability among the given set of standardized signals as a function of the number of PC retained. The red line shows results for the set of UPDRS III measures. The 1^{st} PC captures around 40% of the variability in the data. An analysis of the PCs shows that the 1^{st} PC has large positive contributions from all of the UPDRS3 III measures except for the two related to tremor. Additionally, the first 5 PCs capture ˜80% of the variability. Although clinicians often focus on a single UPDRS III score, not surprisingly, this data provides evidence that there is additional variability in the UPDRS III measures that is not explained by this score alone.

The two blue lines show the results for the motion analysis system metrics for the 1st and 2nd evaluations. The 1st PC alone explains ˜20% of the total variability in these measures. An analysis of the PCs associated to the motion analysis system metrics from the 1^{st} evaluation shows that the 1st component has the largest positive contributions from task timing in flexion/extension and both hand opening/closing tasks and large negative contributions from the mean speed, peak speed and smoothness of flexion-extension movements and the mean jerk during walking, and about 9 dimensions are required to capture around 80% of the variability in these measures. Similar results were obtained for the PCs associated to the motion analysis system metrics from the 2^{nd} evaluation. This suggests that the effective number of independent dimensions associated with the motion analysis system measures is larger than that of the UPDRS III measures.

The green lines show the PCA results when the UPDRS III and motion analysis system measures are combined. The plot for the variance explained as a function of dimension for the combined dataset is consistently close to that of the motion analysis system alone, suggesting that adding data from the motion analysis system increases the number of independent measures beyond what is available from the UPDRS III alone, but that adding the UPDRS III data might not increase the number of independent measures from what is available from the motion analysis system data alone. Examining the 1st PC of the combined dataset, we found the weights associated with the UPDRS III measures are close to the 1st PC of the UPDRS III data alone, and that the weights associated with the motion analysis system measures are close to the 1st PC of the motion analysis system data alone. This suggests that the combination of motion analysis system measures along which variability is maximal may be linearly predictive of the UPDRS III measure.

The optimized model used a combination of 12 predictors from the motion analysis system dataset and was able to predict 83% of the variability in the UPDRS III measure across the patient population (determined from just 6 motion analysis system motor tasks). The motion analysis system signals with the largest weights in the fit model included both hand opening/closing times (which are also assessed as part of UPDRS III exams) and jerk as recorded from the accelerometer mounted on L5 during walking (which is not directly addressed during UPDRS III exams).

Taken together, the PCA and modeling analysis suggest that the motion analysis system signals contain much of the information present in the UPDRS III data and can be used predictively, and contain additional information not present in the UPDRS III data which would be useful in identifying symptom patterns not typically captured in classic exams.

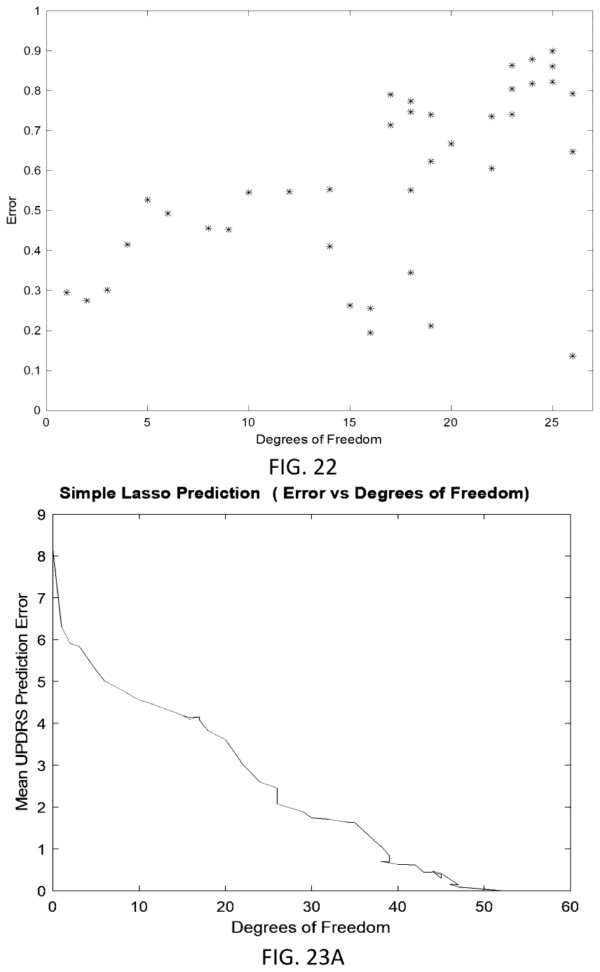

As another example, we conducted an analysis of a group of 29 patients, following the same paradigm described in the previous example, where motion analysis assessments were made on the same day of the UPDRS3 assessment of the patients in the ‘On’ state. In the analysis we used LASSO optimization to explore whether we could predict UPDRS3 assessments based on the motion analysis assessments taken from the same patients. We evaluated the LASSO models performance using a leave-one-out validation procedure. FIG. 22 shows the prediction error of each LASSO model as a function of its number of degrees-of-freedom. In FIG. 22 the prediction error was calculated as 1−mean (mse/variance(Y1)), where mse was calculated as the square of the difference between Y1 and the model prediction, and Y1 was a vector containing the UPDRS3 scores of the patients.

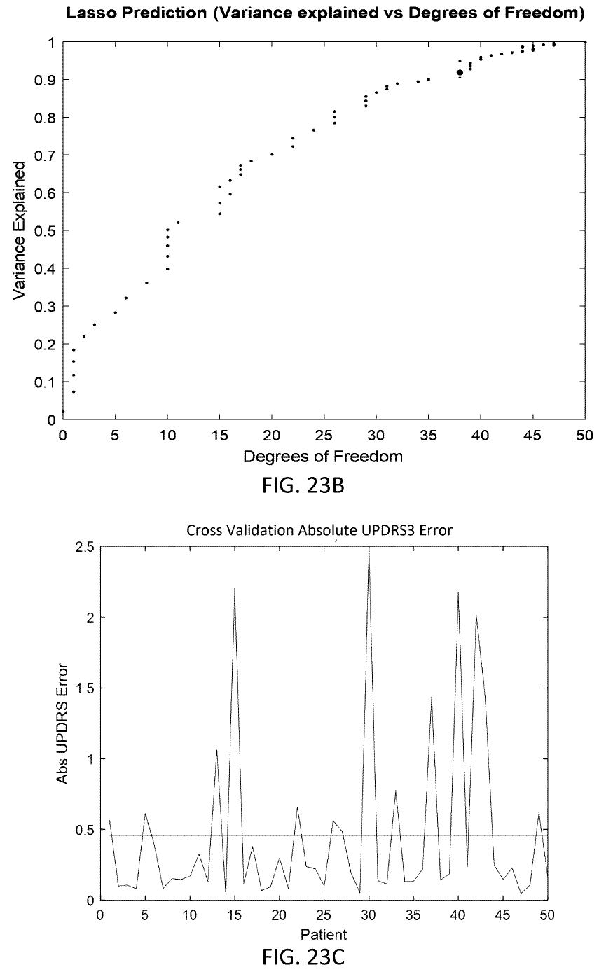

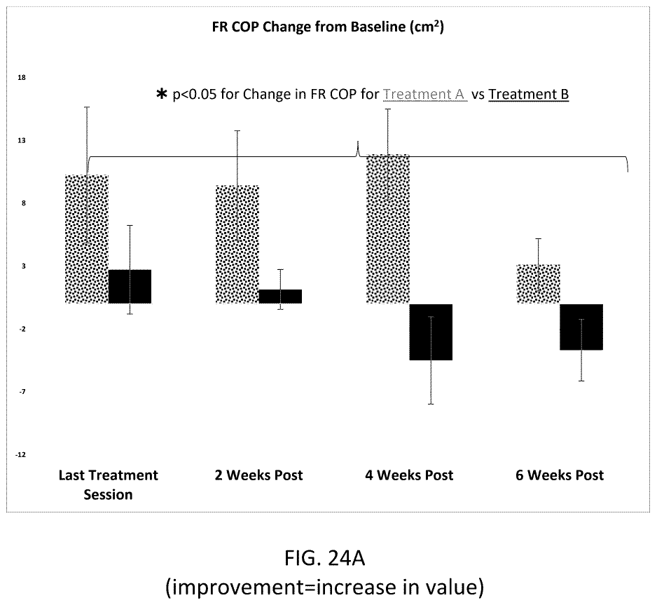

As another example we conducted an analysis of a group of 50 PD patients, following the same paradigm described in the previous example whereby motion analysis system assessments were made on the same day the UPDRS3 assessments of the patients in the ‘On’ state. In the analysis, we implemented a LASSO (while varying the alpha value (penalty term)) to explore individual models with varying degrees of freedom (based on the metrics derived from the motion analysis system), from 1 onward to predict the patients UPDRS3. While we used the LASSO methodology, other linear model techniques can be implemented such as Ridge regression. Note one could overfit a model, but controls are put in place to prevent overfitting (e.g., k-fold cross-validation, early stopping, training with a larger data set, removing features, and early stopping), such as for example herein we choose to set the degrees of freedom to be smaller than the patient group size by a set amount. We further compared the different models that were developed, with different degrees of freedom (of motion analysis suite metrics), model parameters, model types, and/or different motion analysis system metrics, such as by comparing criteria such as the Mean Square Error, R², or total error in predicted UPDRS3 vs the actual UPDRS3 determined in clinical exams (see FIGS. 23A and 23B). The method allowed us to then pick a model with an acceptable number of degrees of freedom and/or metrics derived from the motor evaluations completed with the motion analysis system with the best predictive value of the UPDRS3 (e.g., based on the error metric used). In addition to assessing the predictive accuracy of the model, one could use other criteria, either alone or in combination, such as for example the time for the models to run, computational resources necessary for the models to run, and/or cost of computational methods. The predictive model could in turn be implemented for future predictions with a motion analysis system(s) or be used in part to develop a second model which could implement the optimal metrics identified in the first model, such as for example deriving a second generalized linear model of prediction. We used to the LASSO based metrics identified with the lowest average absolute predicted UPDRS error across the group, determined via cross validation, to in turn develop a generalized linear model. The model itself can be tested or assessed as above, such as through a complete leave-one-out cross validation prediction quality, on the data set (see for example FIG. 23C where we completed such an assessment based and depict the UPDRS3 error in prediction). Note the error levels are within the range or much below that of a motor disorder expert and show the applicability of our method, where in FIG. 23C the mean error across the 50 patients was less than 0.5 UPDRS3 points (Post et. al. Unified Parkinson's disease rating scale motor examination: are ratings of nurses, residents in neurology, and movement disorders specialists interchangeable? 2005 PMID: 16116612). The predictive model(s) can be developed form data which was assessed and improved with methods put in place that could impute missing data should the patients not have completed all tests used in the model (and/or other data used as part of the predictive process, e.g., additional clinical exams), such as implementing different intention to treat methods across the 50 patients, e.g., single or multiple imputation methods (e.g., imputing mean values, imputing median values, imputing most frequent values, imputing zero values, imputing constant values, imputing nearest neighbor values, imputing Multivariate Imputation by Chained Equation, random forest imputation, parametric imputation). One could conduct a model of the models and evaluate the different methods used for prediction, ultimately choosing the optimized methodology as a function of the patient data set and the criteria being predicted (e.g., UPDRS3). As for all the examples herein and methods/systems described, one could implement a big data approach and/or an adaptive model approach where ongoing evaluations from large numbers of patients and/or assessments can be continually implemented to continually improve the prediction process. Such a method can be integrated with other patient data sets to further optimize the prediction methods (e.g., EEG, MRI, EKG, patient history, behavioral assessments, clinical exam data, cognitive assessments). Such as for example, numerous motion analysis systems can be connected via the internet but deployed to multiple clinical sites where stroke patients are assessed and provided physical/rehabilitation therapy. The connected systems could initially assess a patient's baseline information and be used to collect additional data, such as patient imaging data and/or clinical assessments (e.g., Fugl Meyer, which can be uploaded to a central system which develops mathematical models, such as a linear model or correlation models, describing the relationship between the patient's motor exam values gathered with the motion analysis systems and the clinical assessments and/or imaging data). The model can be used as a predictor of the Fugl Meyer score. The uploaded data or generated models can further be used to classify patients (and/or potentially identify patients that would best respond to specific physical therapy/rehabilitation regimens). As more patients are evaluated and/or the patient(s) or patient(s) begin and/or continue to undergo treatment, further data can be uploaded to the central system and the score prediction, classification, and/or therapy tuning can further be improved and optimized. Furthermore, as the classification, prediction method, and/or treatment can be influenced by this motion analysis system(s) based exams, such as by using feedback, one can continuously optimize the classification, prediction method, and/or treatment process by providing the feedback to those conducting the motion analysis system-based exams. As demonstrated, numerous computational methods can be used to complete such a process, and the methods described herein should be considered exemplary as one skilled in the art would note other such methods can be employed.

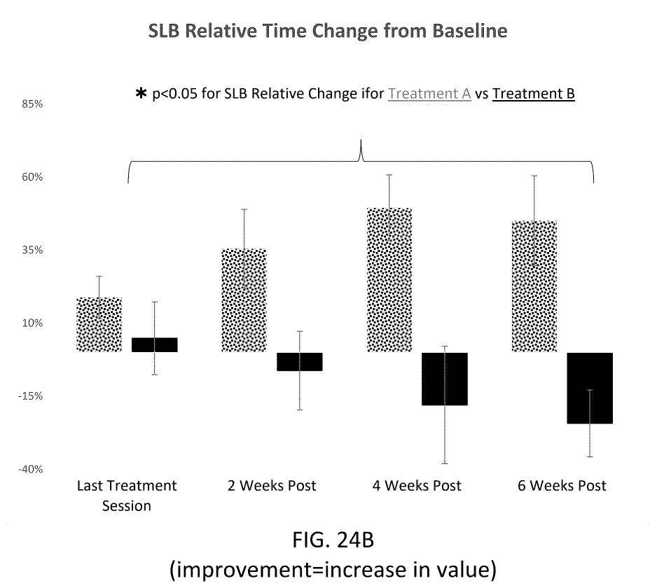

As another example, we examined 20 diabetic neuropathic pain patients undergoing 2 different treatment regimens (10 receiving treatment A and 10 treatment B) while being assessed by a motion analysis system at various time points following their treatments. We used the motion analysis system to assess patient's improvement in Functional Reach (FR) testing, reflected herein as a change in their center of pressure along the reach axis compared to their baseline (pretreatment session). In FIG. 24A we see the summary result of the FR testing and note that the one therapy (Treatment A) has a positive impact on the FR testing, while the other treatment (Treatment B) had a negligible impact on FR testing (it was statistically unchanged from their baseline assessments, but significantly less effective than treatment A). One could use these results to show that the patients improved FR testing (a metric for assessing patients balance and stability) with Treatment A compared to Treatment B (i.e., patients in group B would benefit from therapy A), and for improving patients FR, Treatment A could potentially be repeated at ˜4 weeks. The same patients were also assessed with a motion analysis system while performing a Single Leg Balance (SLB) test, another metric used to assess patient balance and stability, at various time points following their treatments. In FIG. 24B, we see a summary of the testing as assessed with a motion analysis system, focused herein on the relative change in the patients' SLB times. As above, patients that received treatment A improved their performance (compared to pretreatment baseline and compared to treatment B), while patients receiving treatment B had no improvement and had insignificant changes compared to their baseline (pretreatment measure). The data from the motion analysis system be used to tailor one's therapy-herein one would prefer treatment A compared to treatment B for the diabetic neuropathic pain patients for both test metrics if one was tailoring therapy for balance improvements (treatment A could have a higher intensity of brain stimulation for example). One would also note the effects of treatment A improve certain aspects of balance longer than others (reflected in the FR improvements lasting for a less sustained duration than the SLB improvements), and therefor one might want to couple treatment A with other therapies or potentially use increased dosing of the therapy. One could use the motion analysis system to tune the treatment to individual metrics in individual patients.

What is claimed is:

1. A method of determining a management plan for a patient with a movement disorder, the method comprising:
providing a motion analysis system;
obtaining kinematic and/or kinetic information of a patient with a movement disorder using the motion analysis system while the patient is performing a task;
determining biomechanical patterns of the patient based on the obtained kinematic and/or kinetic information; and
determining a management plan for the patient based on the biomechanical patterns.

2. The method according to claim 1, wherein the management plan comprises at least one of changes to an existing therapy regimen, generation of a new therapy regimen, guidance on physical therapy, guidance on movement types to be performed while the patient is performingan activity, or combinations thereof.

3. The method according to claim 1, further comprising:
obtaining additional kinematic and/or kinetic information of the patient at a subsequent point in time; and
updating the management plan based on the additional kinematic and/or kinetic information.

4. The method according to claim 1, wherein the task is selected from the group consisting of: discrete flexion of a joint; discrete extension of a joint; continuous flexion of a joint; continuous extension of a joint; flexion of a joint; extension of a hand; walking; abduction of a joint, adduction of a joint, rotation of a joint, circumduction, pronation, supination, deviation, rotation, stabilizing a joint, reaching, grasping, flexion, extension, abduction, adduction, medial (internal) rotation, lateral (external) rotation, circumduction, pronation, supination, radial deviation (or radial flexion), ulnar deviation (or ulnar flexion), opposition, reposition, dorsiflexion, plantarflexion, inversion, eversion, walking, running, pivoting, leg swing, arm swing, bending, reaching, twisting, sitting to standing, standing, squatting, holding a prone position, holding a static position, lying to sitting, stepping up or down, weight shifting, postural sway, tilting, turning, nodding, pushes or pulls, carrying or lifting, walking on slippery or uneven surfaces, visual challenges, dual-tasking, or combinations thereof.

5. The method according to claim 1, wherein the movement disorder is selected from the group consisting of: Multiple Sclerosis, Amyotrophic Lateral Sclerosis, Alzheimer's Disease, Tics, Parkinson's Disease, Huntington's Disease, Muscular Dystrophy, Cerebral Palsy, Stroke, Myasthenia Gravis, Peripheral Neuropathy, Ataxia, Friedreich's Ataxia, Dystonia, Restless Leg Syndrome, Polio (Poliomyelitis), Guillain-Barré Syndrome, Post-Polio Syndrome, Rheumatoid Arthritis, Osteoarthritis, Lupus, Tardive Dyskinesia, Chorea, Hemiballismus, Wilson's Disease, Brachial Plexus Injury, Tetanus, Motor Neuron Disease, Bell's Palsy, Essential Tremor, Orthostatic Tremor, Rett Syndrome, Spinocerebellar Ataxia, Spinal Muscular Atrophy, Primary Lateral Sclerosis (PLS), Charcot-Marie-Tooth Disease, Complex Regional Pain Syndrome (CRPS), Fibromyalgia, Progressive Supranuclear Palsy, Myoclonus, Phantom Limb Pain, Syringomyelia, Trigeminal Neuralgia, Osteoporosis, Ankylosing Spondylitis, Gout, Paget's Disease of Bone, Lyme Disease, Botulism, Tourette's Syndrome, Prion Diseases, Creutzfeldt-Jakob Disease, Stiff Person Syndrome (SPS), Dermatomyositis, Scleroderma, Batten Disease, Narcolepsy, Chronic Fatigue Syndrome (CFS), Machado-Joseph Disease, Benign Essential Blepharospasm, Foot Drop, Carpal Tunnel Syndrome, Peripheral Artery Disease, Reflex Sympathetic Dystrophy Syndrome, Pantothenate Kinase-Associated Neurodegeneration (PKAN), Mitochondrial Myopathies, Paraneoplastic Syndromes of the Nervous System, Chronic Inflammatory Demyelinating Polyneuropathy (CIDP), Progressive Multifocal Leukoencephalopathy, Transverse Myelitis, Myotonic Dystrophy, Cervical Spondylosis, Behçet's Disease, Pseudotumor Cerebri, Krabbe Disease, Neurofibromatosis, Acoustic Neuroma, Vestibular Neuritis and Labyrinthitis, Vertigo, Meniere's Disease, Chronic Paroxysmal Hemicrania, Antiphospholipid Syndrome (APS), Neuralgia, Paralysis, Postural Orthostatic Tachycardia Syndrome (POTS), Shy-Drager Syndrome, Vasculitis, Hemifacial Spasm, Isaacs' Syndrome, Marfan Syndrome, Osteogenesis Imperfecta, Ehlers-Danlos Syndromes, Alkaptonuria, Spasticity, Athetosis, Hyperkinesias, Hypokinesias, Meralgia Paresthetica, Restless Arms Syndrome, Piriformis Syndrome Spinal Cord Injury, Traumatic Brain Injury, Brain Injury, Diabetes, Cardiovascular Condition, Pulmonary Condition, Balance Ailment, Impingement Syndromes, Joint Replacement, Bone Fusion, bone fracture, joint injury, Trauma, Peripheral Nerve Injury, Post Surgery Injury, Declined Motor Performance, Stuttering, Spasticity, Parkinsonianism, Catatonia, Post-Traumatic Stress Disorder, Stroke, Cognitive Decline, Motor Dysfunction, Motor Performance Decline, Autism, Chronic Pain Syndrome, Epilepsy, Stroke, Auditory Hallucinations, Movement Disorders, Neurodegenerative Disorders, Pain Disorders, Metabolic Disorders, Addictive Disorders, Psychiatric Disorders, Traumatic Nerve Injury, and/or Sensory Disorders.

6. The method according to claim 1, further comprising:
performing physical therapy on the patient based on the therapy management plan;
obtaining additional kinematic and/or kinetic information of the patient at a subsequent point in time; and
updating the therapy management plan based on the additional kinematic and/or kinetic information.

7. The method according to claim 1, wherein the kinematic and/or kinetic information is obtained while the patient is performing at least one of upper limb motor tasks, lower limb motor tasks, walking, standing still, or combinations thereof.

8. The method according to claim 7, wherein the kinematic and/or kinetic information assesses at least one of bradykinesia, tremor, postural instability, or gait.

9. A method for assessing a subject, the method comprising:
obtaining individual kinematic and/or kinetic information of a subject, wherein the kinematic and/or kinetic information of the subject is generated from a motion analysis system;
obtaining population kinematic and/or kinetic information from a population of subjects that present with similar kinematic and/or kinetic information as that of the subject, wherein the kinematic and/or kinetic information of each member of the population is generated from a motion analysis system; and
assessing the subject based on a combination of the individual kinematic and/or kinetic information and the population kinematic and/or kinetic information.

10. The method according to claim 9, wherein assessing comprises diagnosing the subject with a movement disorder.

11. The method according to claim 9, wherein assessing comprises determining severity of an existing movement disorder of the subject.

12. The method according to claim 11, wherein the method is performed at least one additional time at a later point in time.

13. The method according to claim 1, wherein prior to the obtaining step, the method further comprises providing stimulation of neural tissue of the subject.

14. The method according to claim 13, wherein the method is repeated after the subject has received stimulation of their neural tissue.

15. The method according to claim 13, wherein the stimulation is non-invasive transcranial stimulation.

16. The method according to claim 15, wherein the stimulation comprises a combination of electrical and mechanical stimulation.

17-20. (canceled)

21. A system comprised of at least one motion analysis system connected to a central computer wherein the motion analysis system comprises:
at least one sensing device configured to obtain and transmit at least one set of motion data;
at least one synchronization clock; and
wherein the central computer contains:
a central processing unit (CPU) with storage coupled to the CPU for storing instructions that when executed by the CPU cause the CPU to:
receive the set of motion data from the motion analysis system; and
whereby the CPU is further configured to determine a management plan for the patient based on the set of motion data.

22-24. (canceled)
